SEQUENCE LISTING (31 sequences)

SEQ ID NO: 1 - protein, 437 aa, Mus musculus
MLLLLARCFLVILASSLLVCPGLACGPGRGFGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLRPGDRVLAADDQGRLLYSDFLTFLDRDEGAKKVFYVIETLEPRERLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 2 - DNA, 1314 bp, Mus musculus
atgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaagctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctga

SEQ ID NO: 3 - protein, 695 aa, Mus musculus
MAETKIIYHMDEEETPYLVKLPVAPERVTLADFKNVLSNRPVHAYKFFFKSMDQDFGVVKEEIFDDNAKLPCFNGRVVSWLVLAEGAHSDAGSQGTDSHTDLPPPLERTGGIGDSRPPSFQPNVASSRDGMDNETGTESMVSHRRERARRRNRDEAARTNGHPRGDRRRDLGLPPDSASTVLSSELESSSFIDSDEEDNTSRLSSSTEQSTSSRLVRKHKCRRRKQRLRQTDRASSFSSITDSTMSLNIITVTLNMERHHFLGISIVGQSNDRGDGGIYIGSIMKGGAVAADGRIEPGDMLLQVNDVNFENMSNDDAVRVLREIVSQTGPISLTVAKCWDPTPRSYFTIPRADPVRPIDPAAWLSHTAALTGALPRYGTSPCSSAITRTSSSSLTSSVPGAPQLEEAPLTVKSDMSAIVRVMQLPDSGLEIRDRMWLKITIANAVIGADVVDWLYTHVEGFKERREARKYASSMLKHGFLRHTVNKITFSEQCYYVFGDLCSNLASLNLNSGSSGASDQDTLAPLPHPSVPWPLGQGYPYQYPGPPPCFPPAYQDPGFSCGSGSAGSQQSEGSKSSGSTRSSHRTPGREERRATGAGGSGSESDHTVPSGSGSTGWWERPVSQLSRGSSPRSQASAVAPGLPPLHPLTKAYAVVGGPPGGPPVRELAAVPPELTGSRQSFQKAMGNPCEFFVDIM

SEQ ID NO: 4 - DNA, 3286 bp, Mus musculus
agcccgaggggcggcgggccgcgggagccctcagagccgctttccctggcgcccgctccggggccgcggcggatgggcggccgcgggccgcggggcgacaggcggggaacgggtgcgagccgggaccgggaggggcggccgcgccaaggggccgcggggcggccgggcggggcgcgggccggcggtttgggagggcgccccgcgtccgagaggcgagccgggccctgacgccgcgcgggttccgcgtcgcccctgccgcgccatggcggagaccaaaatcatctaccacatggacgaggaggagacgccgtacctggtcaagctgcccgtagctcccgagcgcgtcacgctggccgacttcaagaacgtgctcagcaaccggccggtgcacgcctacaaattcttcttcaagtccatggaccaggacttcggggtggtgaaggaggagatcttcgatgacaatgccaagttgccctgcttcaatggccgggtggtttcctggctggtcctggctgagggcgctcactcggatgcagggtcccagggcactgacagccacacggacctgcccccaccccttgagaggacaggcggcattggggactccaggcccccctccttccaaccaaatgttgccagtagccgggacggaatggacaatgagacaggcacagagtctatggtcagtcaccggcgggagcgagcccgacgtcgaaaccgcgatgaggctgcccggaccaatgggcacccgagaggggatcggcggcgggacctgggactacctccagacagtgcatctactgtactgagcagtgagcttgaatctagcagctttattgactcagatgaggaggacaatacgagccggctgagcagctccacagagcagagcacctcctctcggctagttcggaagcacaaatgccgtcgtcggaagcagcgcttgaggcagacagaccgggcatcctccttcagcagcatcacagactccaccatgtccctgaacatcatcaccgtcactctcaacatggagaggcaccacttcctgggcatcagcatcgtgggccagagcaacgaccggggtgatggcggcatctacattggatccatcatgaagggcggggccgtggctgctgatggccgcattgagccgggcgacatgttgctgcaggtgaacgatgtcaactttgagaacatgagcaatgacgacgctgtacgggtgcttcgggagatcgtgtcccagacagggcccatcagtctcacagtggccaagtgctgggacccaacccctcggagctacttcaccatcccaagggctgacccagtgcgacccatcgacccggctgcctggctgtcccacacagcagcactgacgggtgccctgccccgctatggtacgagtccctgctccagcgccatcacacgcaccagctcttcctcactaaccagctcagtgcctggcgccccacagcttgaggaggcgccgctgactgtgaagagtgacatgagtgccattgtccgcgtcatgcagttgccagactcaggactggagatccgggaccgcatgtggcttaagatcaccattgccaatgctgtcattggggcggatgtggtggactggctgtacacacacgtggagggcttcaaggagcgaagggaggcaagaaagtatgccagcagtatgctgaagcacggtttcctgaggcacaccgtgaacaagatcaccttttctgagcagtgctactatgtctttggcgacctgtgcagtaacctcgcatccctgaacctcaacagtggctccagtggagcctcagatcaggacacactggccccactgccccacccatcagtaccctggcccttgggtcaaggctacccctaccagtacccaggacccccgccctgcttcccacctgcttaccaggaccctggcttcagctgcggcagcggcagtgctgggagccagcagagtgaagggagcaagagcagtgggtccacacggagcagccatcggaccccaggccgagaggagcgccgggcaactggagctgggggtagtggcagtgaatcagaccacacagtaccaagtgggtctggtagcaccggctggtgggagcggcctgtcagccagcttagccgtggcagtagccctcgaagtcaggcttcagctgttgccccagggctccccccactgcacccccttacaaaggcctatgcagtagtgggtgggccacctggagggccacctgtccgggagctggctgctgtccctccagaacttacaggtagccgccagtccttccaaaaggccatgggaaacccctgtgagttctttgtggacatcatgtgatgatcaaccaatgtcttcagcgctgcctgtggctgagtctgagctcctgctgtgccaggagctctgcgctggccgtggtggtggccagccaggatagatcagctgtggggtctgggccagggcagagggagcaggctccagaggaggggcagagggcagccataccaccaggatattggcttgacattttgtctgctcttggggctgctgatggtggtacagctcaagtatctatagagtcttgtaaggagacatctctgactttaagtcctcagcacaagtctcagggaccacctcctggttcccttctttggaagtgacctccatttagaacaagaaaggctctcttgggctcttggtaccccttgccccaaagcctcacagaactgtgcacagggacacaggctgactgtcgctaagttcatgggcctcacctgtcaggccaaggtgggatttttgagggttagacagaaccttcaaacctctctggctgcccaggtggggtctaacttatttatttattgctagcctgcctgctctaagggtggcagctggttacccaaaggggcagtttgcatgcccctttccccacctgctacttggcacatgacaacacagtttgtactgaaggtatgtgaagggtagctagtaggagagacaggagagagacctggcacctagccactgtctcagtctcagtggtgggtgacagtgaacacaagagctgcagaggtgggaccctgttctgtttctgttctggtggctgcccatcatcacgtgccactgccatcccggcacagcggccccacacatctacactagacactgtgtcaaagtctgagtgactgggtagttgacatagagctgcttctgtgtaaatgctgcttctgtgtaaatgctattttaaacactaaaaaagcgtttaattttatggga

SEQ ID NO: 5 - protein, 700 aa, Mus musculus
MLKMLSFKLLLLAVALGFFEGDAKFGERSEGSGARRRRCLNGNPPKRLKRRDRRVMSQLELLSGGEILCGGFYPRVSCCLQSDSPGLGRLENKIFSATNNSECSRLLEEIQCAPCSPHSQSLFYTPERDVLDGDLALPLLCKDYCKEFFYTCRGHIPGLLQTTADEFCFYYARKDAGLCFPDFPRKQVRGPASNYLGQMEDYEKVGGISRKHKHNCLCVQEVMSGLRQPVSAVHSGDGSHRLFILEKEGYVKILTPEGELFKEPYLDIHKLVQSGIKGGDERGLLSLAFHPNYKKNGKLYVSYTTNQERWAIGPHDHILRVVEYTVSRKNPHQVDVRTARVFLEVAELHRKHLGGQLLFGPDGFLYIILGDGMITLDDMEEMDGLSDFTGSVLRLDVDTDMCNVPYSIPRSNPHFNSTNQPPEVFAHGLHDPGRCAVDRHPTDININLTILCSDSNGKNRSSARILQIIKGRDYESEPSLLEFKPFSNGPLVGGFVYRGCQSERLYGSYVFGDRNGNFLTLQQSPVTKQWQEKPLCLGASSSCRGYFSGHILGFGEDELGEVYILSSSKSMTQTHNGKLYKIVDPKRPLMPEECRVTVQPAQPLTSDCSRLCRNGYYTPTGKCCCSPGWEGDFCRIAKCEPACRHGGVCVRPNKCLCKKGYLGPQCEQVDRNVRRVTRAGILDQIIDMTSYLLDLTSYIV

SEQ ID NO: 6 - DNA, 2669 bp, Mus musculus
gctgcagccgccggcagaggagacctcagcatcctcgggagcccagcgccgaccctgcctccgcccggcccgctgccgccaccgccgccctttcggttcctgctactgtctcacctaaacaactcccattcagcggacaagcgaagttctatgactctcttctcctctccttcctcctcttcttccaactccttctcctcccacttcccaaccgctgtggaaagcccctaacccaacagacgctggcaaggctgcggacaagtgtcaactgcactttatcttgctgctcctactgccctaaggcaaagttgcatagctctacatctttctttcccagccacctccctctgcccccaagagcgtcccgccgccccgcagcactctcctggagctgcgccctagtgcccctgctgggcagtggcctttcccccaccccatcctcccgcgtcctgcccttgctgctccgggcagacgatgctgaagatgctctcgtttaagctgctactgctggccgtggctctgggcttctttgaaggagatgcgaagtttggggaaaggagcgaggggagcggagcgagaaggagacggtgcctgaatgggaaccccccaaagcgcctaaagagaagggacaggcgggtgatgtcccagctggagctgctcagtggaggagagatcctgtgtggtggcttctacccacgagtatcttgctgcctgcagagtgacagccctggattggggcgtctggagaacaagatcttttctgccaccaacaactcagaatgcagcaggctgctggaggagatccaatgtgctccctgctccccgcattcccagagcctcttctacacacctgaaagagatgtcctggatggggacctagcacttccgctcctctgcaaagactactgcaaagaattcttttatacttgccgaggccatattccaggtcttcttcaaacaactgctgatgaattttgcttttactatgcaagaaaagatgctgggttatgctttccagacttcccgagaaagcaagtcagaggaccagcatctaactacttgggccagatggaagactacgagaaagtgggggggatcagcagaaaacacaaacacaactgcctctgtgtccaggaggtcatgagtgggctgcggcagcctgtgagcgctgtgcacagcggggatggctcccatcggctcttcattctagagaaggaaggctacgtgaaaattctaaccccagaaggagaactgttcaaggagccttacttggacattcacaaacttgttcaaagtggaataaagggaggagacgaaaggggcctgctaagcctggcattccatcccaattacaagaaaaatggaaagctgtatgtgtcttataccaccaaccaggaacggtgggctattgggcctcacgaccacattcttcgggttgtggaatacacagtatccaggaaaaacccccatcaagttgatgtgagaacagccagggtgtttctggaagtcgcagagctccaccgaaagcatcttgggggacagctgctctttggtcctgatggctttttgtacatcatccttggggatggtatgatcacattggatgacatggaagagatggatgggttaagtgacttcacaggctctgtgctgaggctggacgtggacaccgacatgtgcaatgtgccttattccatacctcggagtaaccctcacttcaacagcaccaaccagcccccagaagtatttgcccacggcctccatgatccaggcagatgtgccgtggatcgacatcctactgatataaacatcaatttaacaatactttgctcagattccaacgggaaaaacaggtcatcagccagaatcctacagataataaagggaagagattatgaaagtgagccatctcttcttgaattcaagccattcagtaacggccctttggttggtggatttgtttacagaggctgtcagtctgaaagattgtacggaagctatgtgttcggagatcgcaatgggaatttcttaaccctccagcaaagcccagtgaccaagcaatggcaagaaaagccgctctgcctgggtgccagcagctcctgtcgaggctacttttcgggtcacatcttgggatttggagaagatgaattaggagaggtttacattctatcaagcagtaagagtatgacccagactcacaatggaaaactctacaagatcgtagaccccaaaagacctttaatgcctgaggaatgcagagtcacagttcaacctgcccagccactgacctccgattgctcccggctctgtcgaaacggctactacacccccactggcaagtgctgctgcagtcccggctgggagggagacttctgcagaattgccaagtgtgagccagcgtgccgtcatggaggtgtctgtgtcagaccgaacaagtgcctctgtaaaaagggctatcttggtcctcaatgtgaacaagtggacaggaacgtccgcagagtgaccagggcaggtatccttgatcagatcattgacatgacgtcttacttgctggatctcacaagttacattgtatagtttctgggacagttcgagtctatctttccagtgggcatttattttgaccttgtcatcattaaaaagagagactgtccttctgctacaaaaaa

SEQ ID NO: 7 - protein, 379 aa, Mus musculus
MARRRAFPAFALRLWSILPCLLLLRADAGQPPEESLYLWIDAHQARVLIGFEEDILIVSEGKMAPFTHDFRKAQQRMPAIPVNIHSMNFTWQAAGQAEYFYEFLSLRSLDKGIMADPTVNVPLLGTVPHKASVVQVGFPCLGKQDGVAAFEVNVIVMNSEGNTILRTPQNAIFFKTCQQAECPGGCRNGGFCNERRVCECPDGFYGPHCEKALCIPRCMNGGLCVTPGFCICPPGFYGVNCDKANCSTTCFNGGTCFYPGKCICPPGLEGEQCELSKCPQPCRNGGKCIGKSKCKCPKGYQGDLCSKPVCEPGCGAHGTCHEPNKCQCREGWHGRHCNKRYGASLMHAPRPAGAGLERHTPSLKKAEDRRDPPESNYIW

SEQ ID NO: 8 - DNA, 2047 bp, Mus musculus
agtaggaacagctccagccccgccagctgcagccaaggcgagaacttcacaagcagcacaggttgggtcgctgcggcaggagttgcaccaccagcgagaaggtcctgagcaccatggctcggagaagagccttccctgctttcgcgctccggctctggagcatcctaccttgcctgctcctgctgcgagcggatgcagggcagccacctgaggagagcttgtacctgtggatcgacgcccatcaggctagagtgctcataggatttgaagaagacattctgattgtctcggaggggaaaatggccccctttacacatgatttcaggaaagcccaacaaagaatgccagccattcctgtcaatatccactccatgaattttacctggcaagctgcggggcaggcagaatacttctacgagttcctgtctctgcgctccctggataaaggcatcatggcagatccaactgtcaatgtccctttgctgggaacagtgcctcacaaggcatcagttgttcaagttggtttcccgtgtctcggcaaacaagacggggtagcagcatttgaagtgaatgtgattgtcatgaattctgaaggcaacaccatccttaggacccctcagaatgccatcttctttaaaacatgtcaacaagctgagtgtcccggagggtgtcgaaatggaggcttttgtaacgaaaggcgggtctgcgagtgtccggatgggttctacgggcctcactgtgagaaagccctgtgcataccccgatgtatgaacggtggtctgtgtgtcactcctggcttctgcatctgcccccctggattctacggtgtcaactgtgacaaagcaaactgctcaaccacctgctttaatggagggacctgcttttacccgggaaaatgtatttgccctcctggactcgagggagagcagtgtgaactcagcaaatgcccccaaccctgccgaaatggaggtaaatgcattggtaaaagcaagtgtaagtgcccgaaaggttaccaaggagacctgtgctctaagcccgtctgcgagcctggctgtggtgcccacggaacctgccacgaacccaacaagtgccagtgtcgagagggctggcacggcagacactgcaataagaggtatggagccagcctcatgcatgccccgaggccagcaggcgccgggctggagcgacacacgccttcacttaaaaaggctgaggatagaagggatccacctgaatccaattacatctggtgaacccctaccccaccatctgaaacggttcaagttacaccgggttcacagcctttgttaacctttcgcgtgttggatgttcaaatgctgttcattacactttagaacgccggcctgaattttattagcttcattataaatcactgggctgatatctactcttccttttaggttttctaagcgtgtctagcatgatggtatagattttcttccttcagtccttttgggacagatcttatattgtgtcagttgatcaggttaaaaagaaaaaaaaatatctgtcttttcagtgtgtagttgacagatacttgcaaaatcacaacacatttgtggtcttagaatggggagtgttagagaggttaaactgggcagagatgcataaattacaaggtttcggataaagccaatagcagcgtttaagctacagtatttccaattttattgtcaaatatttggacatctgtctaattaatacttcaattgcccccccccccatcttgaatgcatacaatctatttcacccttgctgttactctagacagttcagttttgatggggcgggggacaaagtttaaaaaaattacactgagttagcggcatttaaacaatataatatattgtaaacacgacgagataaggaatataatgtatgaagcctttgcattggatggaagcaatataatatattgtaaacaaaacacagctcttacatagtaaacgttttatactgtttgtatgtatgaaataaaggtgacgctttcactttcaaaaaaaaaaaaaaaaaaaaaaaaaaaaaa

SEQ ID NO: 9 - DNA, 24 bp, Artificial Sequence
  Description of Artificial Sequence Synthetic oligonucleotide
tcgtcgttttgtcgttttgtcgtt

SEQ ID NO: 10 - DNA, 19 bp, Artificial Sequence
  Description of Artificial Sequence Synthetic primer
ccaccaactgggacacatg

SEQ ID NO: 11 - DNA, 24 bp, Artificial Sequence
  Description of Artificial Sequence Synthetic primer
gtctcaaacatgatctgggtcatc

SEQ ID NO: 12 - DNA, 18 bp, Artificial Sequence
  Description of Artificial Sequence Synthetic primer
agggggtttggaaagagg

SEQ ID NO: 13 - DNA, 22 bp, Artificial Sequence
  Description of Artificial Sequence Synthetic primer
ggattcatagtagacccagtcg

SEQ ID NO: 14 - DNA, 18 bp, Artificial Sequence
  Description of Artificial Sequence Synthetic primer
atcggagtggagttcacc

SEQ ID NO: 15 - DNA, 20 bp, Artificial Sequence
  Description of Artificial Sequence Synthetic primer
ctgctgtgcttcgtattgcc

SEQ ID NO: 16 - DNA, 22 bp, Artificial Sequence
  Description of Artificial Sequence Synthetic primer
catcaagttcaacagttcagga

SEQ ID NO: 17 - DNA, 30 bp, Artificial Sequence
  Description of Artificial Sequence Synthetic primer
ataggtgaggaccacgaaccacactactcc

SEQ ID NO: 18 - DNA, 18 bp, Artificial Sequence
  Description of Artificial Sequence Synthetic primer
gagaagccacacaagtgc

SEQ ID NO: 19 - DNA, 22 bp, Artificial Sequence
  Description of Artificial Sequence Synthetic primer
aacagtcagtctgctctcttcc

SEQ ID NO: 20 - DNA, 22 bp, Artificial Sequence
  Description of Artificial Sequence Synthetic primer
tgacccctttagcctacaagca

SEQ ID NO: 21 - DNA, 26 bp, Artificial Sequence
  Description of Artificial Sequence Synthetic primer
ttcttgtgatcttcccttcatatctg

SEQ ID NO: 22 - DNA, 28 bp, Artificial Sequence
  Description of Artificial Sequence Synthetic primer
tttattcccaacgtagccgagaagaccc

SEQ ID NO: 23 - DNA, 20 bp, Artificial Sequence
  Description of Artificial Sequence Synthetic primer
ctccaagtgtcgtccggttt

SEQ ID NO: 24 - DNA, 22 bp, Artificial Sequence
  Description of Artificial Sequence Synthetic primer
tgtactccgagtcggaggaatc

SEQ ID NO: 25 - DNA, 24 bp, Artificial Sequence
  Description of Artificial Sequence Synthetic primer
cgtgcctcctggtcacacgaacaa

SEQ ID NO: 26 - DNA, 22 bp, Artificial Sequence
  Description of Artificial Sequence Synthetic primer
ggctgtcggaagtcctattcac

SEQ ID NO: 27 - DNA, 25 bp, Artificial Sequence
  Description of Artificial Sequence Synthetic primer
caaccttcttgctcacacatgtaag

SEQ ID NO: 28 - DNA, 20 bp, Artificial Sequence
  Description of Artificial Sequence Synthetic primer
cgcaccttcggtcgcacacg

SEQ ID NO: 29 - DNA, 18 bp, Artificial Sequence
  Description of Artificial Sequence Synthetic primer
gccctgtgccaccatgtg

SEQ ID NO: 30 - DNA, 21 bp, Artificial Sequence
  Description of Artificial Sequence Synthetic primer
cggtagcgacgagagaagtca

SEQ ID NO: 31 - DNA, 20 bp, Artificial Sequence
  Description of Artificial Sequence Synthetic primer
ccatctgaccctccgccggg

(19) United States
(12) Patent Application Publication
(10) Pub. No.: US 2004/0126359 A1
(43) Pub. Date: Jul. 1, 2004

(54) Hedgehog

(76) Inventors: Jonathan Robert Lamb, Edinburgh (GB); Gerard Francis Hoyne, Canberra (AU); Margaret Jane Dallman, London (GB); Brian Robert Champion, Cambridge (GB)

Correspondence Address: FROMMER LAWRENCE & HAUG, 745 FIFTH AVENUE- 10TH FL., NEW YORK, NY, 10151, US

(21) Appl. No.: 10/682,230

(22) Filed: Oct. 9, 2003

Related U.S. Application Data
(63) Continuation-in-part of application No. PCT/GB02/01666, filed on Apr. 9, 2002.

(30) Foreign Application Priority Data
Apr. 9, 2001 (GB) 0108873.1
Apr. 9, 2001 (GB) 0108872.3

(51) Int. Cl. A61K 38/17; A61K 38/20

(52) U.S. Cl. 424/85.2; 514/12

(57) ABSTRACT

Provided is a method of modulating T-cell activation, proliferation or apoptosis by contacting T-cells with a modulator of a Hedgehog signalling pathway or a modulator of a pathway which is a target of the Hedgehog signaling pathway.

### CROSS-REFERENCE TO RELATED APPLICATIONS

This application is a continuation-in-part of International Application No. PCT/GB02/01666, filed on Apr. 9, 2002, published as WO 02/080952 on Oct. 17, 2002, and claiming priority to GB applications Serial Nos. 0108872.3 and 0108873.1, both filed on Apr. 9, 2001.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to the prevention and treatment of diseases associated with T-cell mediated diseases.

### BACKGROUND OF THE INVENTION

Normal development, growth and homeostasis in multi-cellular organisms require a careful balance between the production and destruction of cells in tissues throughout the body. Cell division is a carefully coordinated process with numerous control mechanisms. These mechanisms are designed to regulate DNA replication and to prevent inappropriate or excessive proliferation. In contrast, programmed cell death is the genetically controlled process by which unneeded or damaged cells can be eliminated without causing the tissue destruction and inflammatory responses that are often associated with acute injury and necrosis.

The term “apoptosis” was first used by Kerr J F et al, (1972) Br. J. Cancer 26:239-257 to describe the morphological changes that characterise cells undergoing programmed cell death. Dysregulation of apoptosis has recently been recognised as a significant factor in the pathogenesis of human disease. For example, inappropriate cell survival can cause or contribute to many diseases such as cancer, autoimmune diseases and inflammatory diseases. In contrast, increased apoptosis can cause immunodeficiency diseases such as AIDS, neurodegenerative disorders and myelodysplastic syndromes.

The discovery of mechanisms controlling the regulation of programmed cell death (hereinafter “apoptosis”) provides a means to investigate and provide diagnostic or therapeutic compositions useful in the detection, prevention and treatment of cancer, autoimmune diseases, lymphoproliferative disorders, psoriasis, atherosclerosis, restenosis, AIDS, immunodeficiency diseases, ischemic injuries, neurodegenerative diseases, osteoporosis, myelodysplastic syndromes, toxin-induced diseases, cachexia and viral infections.

Immunologic tolerance to self antigens is a necessary mechanism for protecting an organism from destruction by its own immune system. When this mechanism fails, allowing self-reactive immune cells to proliferate, an autoimmune disease develops within the host. A number of diseases such as Multiple Sclerosis (MS), Lupis, Myathenia Gravis and Rheumatoid Arthritis (RA) have been shown to result from loss of self-tolerance in T lymphocytes. For example, Myelin reactive T-cells have been demonstrated in patients with MS.

RA is characterised by chronic inflammation of the synovial joints resulting from hyperplasia of synovial fibroblasts and infiltration of lymphocytes, macrophages and plasma cells. All of these cells proliferate abnormally and produce an elevated amount of inflammatory cytokines. Some of the pathophysiological consequences of the disease may be explained by inadequate apoptosis, which may promote the survival of autoreactive T-cells. It has therefore been proposed that induction of apoptosis in the rheumatoid joint can be used to therapeutic advantage in the disease.

There is however a continuing need in the art for additional methods and tools for treating diseases mediated by increased or decreased apoptosis and for T-cell mediated diseases such as autoimmune diseases, as well as allergic diseases and transplantation rejection and cancers.

### SUMMARY OF THE INVENTION

The present invention relates to the discovery of mechanisms controlling T-cell activation and provides a means to investigate and to provide diagnostic or therapeutic compositions useful in the detection, prevention and treatment of diseases or disorders including diseases and infections mediated by T-cells. In particular, we have now shown that Sonic hedgehog (Shh) and Patched (Ptc) protein are expressed by T-cells; Shh can modulate Ptc expression by T-cells; and that Shh can modulate T-cell gene expression patterns. Bhardwaj et al Nature Immun. (2001) 2:172-180 suggested that Shh is an important regulator of primitive hematopoietic cells that is dependent on downstream BMP signals, and although it is known that Hedgehog (HH) may play a role in the survival of T-cells in thymus cells, we believe that we are the first to consider the mammalian peripheral immune system and to connect modulation of the Hedgehog signalling pathway with the treatment of apoptosis and T-cell mediated diseases.

The present invention provides a method of treatment of T-cell mediated diseases and/or diseases in which normal T-cell apoptosis is blocked or increased comprising the administration of a therapeutically effective amount of a modulator of a component of a Hedgehog family member signalling pathway or a modulator of a component of a signalling pathway which is a target of Hedgehog signalling to an individual in need of the same.

In a first aspect of the present invention, there is provided the use of a modulator of a Hedgehog signalling pathway, or a modulator of a pathway which is a target of the Hedgehog signalling pathway in the preparation of a medicament for treatment of a T-cell mediated disease or infection.

In a second aspect of the present invention, there is provided the use of a modulator of a Hedgehog signalling pathway, or a modulator of a pathway which is a target of the Hedgehog signalling pathway in the preparation of a medicament for treatment of a disease or disorder associated with increased or decreased T-cell apoptosis.

In a third aspect of the present invention, there is provided the use of a modulator of a Hedgehog signalling pathway, or a modulator of a pathway which is a target of the Hedgehog signalling pathway in the preparation of a medicament for modification of T-cell activation.

In a fourth aspect of the present invention, there is provided the use of a modulator of a Hedgehog signalling pathway, or a modulator of a pathway which is a target of the Hedgehog signalling pathway in the preparation of a medicament for modification of T-cell proliferation.

In a fifth aspect of the present invention, there is provided the use of a modulator of a Hedgehog signalling pathway, or a modulator of a pathway which is a target of the Hedgehog signalling pathway in the preparation of a medicament for modification of peripheral T-cell activation.

By the term “peripheral” T-cell we include “extra-thymic” T-cells.

In a sixth aspect of the present invention, there is provided the use of a modulator of a Hedgehog signalling pathway, or a modulator of a pathway which is a target of the Hedgehog signalling pathway in the preparation of a medicament for modification of peripheral T-cell proliferation.

In a seventh aspect of the present invention, there is provided the use of a modulator of a Hedgehog signalling pathway, or a modulator of a pathway which is a target of the Hedgehog signalling pathway in the preparation of a medicament for modification of T-cell apoptosis.

In an eighth aspect of the present invention, there is provided the use of a modulator of a Hedgehog signalling pathway, or a modulator of a pathway which is a target of the Hedgehog signalling pathway in the preparation of a medicament for modification of peripheral T-cell apoptosis.

In a nineth aspect of the present invention, there is provided the use of an agonist of a Hedgehog signalling pathway, or an agonist of a pathway which is a target of the Hedgehog signalling pathway in the preparation of a medicament for treatment of a T-cell mediated disease or infection.

In a tenth aspect of the present invention, there is provided the use of an agonist of a Hedgehog signalling pathway, or an agonist of a pathway which is a target of the Hedgehog signalling pathway in the preparation of a medicament for treatment of a disease or disorder associated with increased or decreased T-cell apoptosis.

In an eleventh aspect of the present invention, there is provided the use of an agonist of a Hedgehog signalling pathway, or an agonist of a pathway which is a target of the Hedgehog signalling pathway in the preparation of a medicament for treatment of a disease or disorder associated with increased or decreased T-cell proliferation.

In a twelfth aspect of the present invention, there is provided the use of a modulator of a Hedgehog signalling pathway, or a modulator of a pathway which is a target of the Hedgehog signalling pathway in the preparation of a medicament for modulation of the Notch signalling pathway.

In a thirteenth aspect of the present invention, there is provided the use of a modulator of a Hedgehog signalling pathway, or a modulator of a pathway which is a target of the Hedgehog signalling pathway in the preparation of a medicament for modulation of the Notch signalling pathway in immune cells.

Preferably, the Hedgehog signalling pathway is the Sonic hedgehog, Indian hedgehog or Desert hedgehog signalling pathway and the pathway which is a target of the Hedgehog signalling pathway is the Wnt signalling pathway.

In a preferred embodiment, the modulator is an inhibitor or upregulator of the biological activity of the pathway. Preferably, the inhibitor is HIP, cyclopamine, Frzb, Cerberus, WIF-1, Xnr-3, Gremlin, or Follistatin or a derivative, fragment, variant, mimetic, homologue or analogue thereof. Even more preferably, the inhibitor is Ptc, Cos2 or PKA or an agent of the cAMP signal transduction pathway. Alternatively, the modulator is a member of the TGF-β family such as TGF-β-1 and TGF-β-2, an interleukin such as IL-4, IL-10 and IL-13, IFN-γ, or FLT3 ligand, or a member of the BMP family. In one embodiment, the modulator is an antibody.

In a preferred embodiment, the present invention is used for the preparation of a medicament for the treatment of cancer of the breast, prostate and ovary as well as lymphomas and carcinomas, autoimmune diseases such as systemic lupus erythematosus (SLE), glomerulonephritis, Sjogren's syndrome, Graves disease, MS, RA and diabetes, inflammatory diseases such as osteoarthritis, Crohn's disease, inflammatory bowel disease and colitis, proliferative disorders such as atherosclerosis, restenosis, psoriasis, lymphadenopathy, and viral infections such as by herpesviruses, poxviruses and adenoviruses.

In another preferred embodiment, the present invention is used for the preparation of a medicament for the treatment of AIDS and other infectious or genetic immunodeficiencies, neurodegenerative diseases such as Alzheimer's disease, Parkinson's disease, amyotrophic lateral sclerosis, retinitis pigmentosa and cerebellar degeneration, myelodysplastic syndromes such as aplastic anemia, ischemic injuries such as myocardial infarction, stroke and reperfusion injury, toxin-induced diseases such as alcohol-induced liver damage, cirrhosis and lathyrism, wasting diseases such as cachexia, viral infections such as hepatitis B and C, and osteoporosis.

In another preferred embodiment, the present invention is of use for the preparation of a medicament for the treatment of asthma, allergy, graft rejection, autoimmunity, tumour induced abberrations to the T-cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Preferably, the present invention is used for the the preparation of a medicament for the treatment of multiple sclerosis, rheumatoid arthritis or diabetes.

In a fourteenth aspect of the present invention, there is provided a method for modulating T-cell activation by administering a modulator of a Hedgehog signalling pathway, or a modulator of a pathway which is a target of the Hedgehog signalling pathway.

In a fifteenth aspect of the present invention, there is provided a method for modulating T-cell proliferation by administering a modulator of a Hedgehog signalling pathway, or a modulator of a pathway which is a target of the Hedgehog signalling pathway.

In an sixteenth aspect of the present invention, there is provided a method for modulating peripheral T-cell proliferation by administering a modulator of a Hedgehog signalling pathway, or a modulator of a pathway which is a target of the Hedgehog signalling pathway.

In a seventeenth aspect of the present invention, there is provided a method for modulating T-cell apoptosis by administering a modulator of a Hedgehog signalling pathway, or a modulator of a pathway which is a target of the Hedgehog signalling, pathway.

In a eighteenth aspect of the present invention, there is provided a method for modulating peripheral T-cell apoptosis by administering a modulator of a Hedgehog signalling pathway, or a modulator of a pathway which is a target of the Hedgehog signalling pathway.

In a nineteenth aspect of the present invention, there is provided a method for treating a T-cell mediated disease or infection by administering an agonist of a Hedgehog signalling pathway, or an agonist of a pathway which is a target of the Hedgehog signalling pathway.

In a twentieth aspect of the present invention, there is provided a method for treating a disease or disorder associated with increased or decreased T-cell apoptosis by administering an agonist of a Hedgehog signalling pathway, or an agonist of a pathway which is a target of the Hedgehog signalling pathway.

In a twenty-first aspect of the present invention, there is provided a method for treating a disease or disorder associated with increased or decreased T-cell proliferation by administering an agonist of a Hedgehog signalling pathway, or an agonist of a pathway which is a target of the Hedgehog signalling pathway.

In a twenty-second aspect of the present invention, there is provided a method for modulating the Notch signalling pathway by administering a modulator of a Hedgehog signalling pathway, or a modulator of a pathway which is a target of the Hedgehog signalling pathway.

In a twenty-third aspect of the present invention, there is provided a method for modulating the Notch signalling pathway in immune cells by administering a modulator of a Hedgehog signalling pathway, or a modulator of a pathway which is a target of the Hedgehog signalling pathway. Preferably, the immune cells are peripheral T-cells.

In a twenty-fourth aspect of the present invention, there is provided a composition for use in treatment of T-cell mediated diseases comprising a therapeutically effective amount of a modulator of a Hedgehog signalling pathway or a modulator of a target pathway of the Hedgehog signalling pathway and a pharmaceutically acceptable carrier, diluent or excipient.

In a twenty-fifth aspect of the present invention, there is provided a composition for use in the treatment of diseases associated with increased or decreased T-cell apoptosis comprising a therapeutically effective amount of a modulator of a Hedgehog signalling pathway or a modulator of a target pathway of the Hedgehog signalling pathway and a pharmaceutically acceptable carrier, diluent or excipient.

In a twenty-sixth aspect of the present invention, there is provided a composition for use in the treatment of diseases associated with modification of T-cell activation, T-cell proliferation, peripheral T-cell activation, peripheral T-cell proliferation and T-cell apoptosis comprising a therapeutically effective amount of a modulator of a Hedgehog signalling pathway or a modulator of a target pathway of the Hedgehog signalling pathway and a pharmaceutically acceptable carrier, diluent or excipient.

In a twenty-seventh aspect of the present invention, there is provided a method for detecting modulators of Hedgehog signalling comprising the steps of monitoring Hedgehog signalling in a cell of the immune system in the presence and absence of a candidate modulator, and determining whether the candidate modulator modulates Hedgehog signalling.

In a twenty-eighth aspect of the present invention, there is provided a method for detecting modulators of Hedgehog signalling comprising the steps of:

(a) contacting a cell of the immune system with a candidate modulator;

(b) monitoring Hedgehog signalling; and

(c) determining whether the candidate modulator modulates Hedgehog signalling.

In a preferred embodiment, the candidate modulator is selected from the group consisting of: an organic compound, a inorganic compound, a peptide or polypeptide, a polynucleotide, an antibody, a fragment of an antibody, a cytokine and a fragment of a cytokine.

Preferably, the step of monitoring Hedgehog signalling comprises the step of monitoring levels of expression of at least one target gene. In one embodiment, the at least one target gene is an endogenous target gene of Hedgehog signalling. Alternatively, the at least one target gene is a reporter gene, preferably selected from the group consisting of: a gene encoding a polypeptide having an enzymatic activity, a gene comprising a radiolabel or a fluorescent label and a gene encoding a predetermined polypeptide epitope.

In a preferred embodiment, the at least one target gene is under the transcriptional control of a promoter region sensitive to Hedgehog signalling.

In an even more preferred embodiment, the at least one target gene is under the transcriptional control of a promoter region sensitive to:

i) Hedgehog signalling; and

ii) a second signal; and/or

iii) a third signal

wherein the second and third signals are different.

In one embodiment, the second signal results from activation of a signalling pathway specific to cells of the immune system, preferably a T-cell receptor (TCR) signalling pathway; a B cell receptor (BCR) signalling pathway; or a Toll-like receptor (TLR) signalling pathway.

The third signal is preferably a costimulus specific to cells of the immune system. In a preferred embodiment, the costimulus is selected from the group consisting of: B7 proteins B7.1-CD80, B7.2-CD86, B7H1, B7H2, B7H3, B7RP1, B7RP2, CTLA4, ICOS, CD2, CD24, CD27, CD27L, CD3, CD30, CD30L, CD34, CD38, CD40, CD40L, CD44, CD45, CD49, CD69, CD70, CD95 (Fas), CD134, CD134L, CD153, CD154, 4-1BB, 4-1BB-L, LFA-1, ICAM-1, ICAM-2, ICAM-3, OX40, OX40L, PD-1, PDL1, PDL2, TIM-1, TRANCE/RANK ligands, Fas ligand, MHC class II, DEC205-CD205, CD204-Scavenger receptor, CD14, CD206 (mannose receptor), Toll-like receptors (TLRs), such as TLR 1-11, CD207 (Langerin), CD209 (DC-SIGN), FC-γ receptor 2 (CD32), CD64 (FCγ receptor 1), CD68, CD83, CD33, CD54, BDCA-2, BDCA-3, BDCA-4, chemokine receptors, cytokines, growth factors and growth factor receptor agonists, and variants, derivatives, analogues and fragments thereof.

In a preferred embodiment, the cell of the immune system is an antigen presenting cell (APC), preferably a T-cell or T-cell progenitor, including a peripheral (i.e. extra-thymic) T-cell.

In another preferred embodiment, expression of the at least one target gene is monitored with a protein assay and/or a nucleic acid assay.

In a twenty-ninth aspect of the present invention, there is provided a modulator identifiable by a method of the invention.

In a twenty-fifth aspect of the present invention, there is provided a method for detecting modulators of Hedgehog signalling comprising the steps of:

(a) activating a cell of the immune system;

(b) contacting the cell with a candidate modulator;

(c) monitoring Hedgehog signalling;

(wherein steps (a), (b) and (c) can be carried out in any order); and

(d) determining whether the candidate modulator modulates Hedgehog signalling.

Preferably, the cell of the immune system is a T-cell. In one embodiment, the T-cell is activated by activation of the T-cell receptor. The T-cell receptor may be activated with an antigen or antigenic determinant. Alternatively, the T-cell receptor may be activated by an anti-TCR antibody, preferably an anti-CD3 antibody.

In another embodiment, the T-cell is co-activated. Preferably, the T-cell is co-activated by activation of CD28. The T-cell receptor may be co-activated by an anti-CD28 antibody.

The term “modulate” as used herein refers to a change or alteration in the biological activity of the Hedgehog signalling pathway or a target signalling pathway thereof. In one embodiment the modulator is an “antagonist” or “inhibitor” which blocks, at least to some extent, the normal biological activity of the Hedgehog signalling pathway. Antagonists and inhibitors may include proteins, nucleic acids and may include antibodies. In another embodiment the modulator is an agonist of the Hedgehog signalling pathway or a target signalling pathway thereof.

The terms “comprises”, “comprising”, and the like can have the meaning ascribed to them in U.S. Patent Law and can mean “includes”, “including” and the like.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 shows a schematic representation of HH signalling;

FIG. 2 shows a schematic representation of a component of HH signalling;

FIG. 3 shows a schematic representation of Wnt signalling;

FIGS. 4A and 4B show Shh expression by human T-cells. FIG. 4A shows anti-Shh immuno-staining of unstimulated T-cells. FIG. 4B shows the results of PCR analysis of expression of Shh by CD4+ and CD8+ human T-cells. pSHH=Shh plasmid control;

FIGS. 5A-5D show Ptc expression in human T-cells. FIG. 5(A) shows an analysis of the effect of increasing Shh concentrations on Ptc mRNA levels in unactivated human CD4+ T-cells at 24 hrs using Taqman analysis. FIG. 5(C) shows antibody staining of human T-cells with anti-human Ptc antibodies; FIG. 5(D) shows the results of PCR analysis of expression of Ptc in human CD4 and CD8 T-cells and B cells;

FIG. 6 shows PCR analysis of expression of components of the Hedgehog pathway in peripheral lymphoid tissues: spleen (S), lymph node (LN) and thymus (T). Water (H2O) was used as a negative control.;

FIG. 7 shows proliferation of human CD4 T-cells cultured with Shh N-terminal active peptide (aCD3/aCD28) (measured by reference to incorporation of 3H thymidine);

FIG. 8 shows proliferation of human CD8 T-cells cultured with Shh N-terminal active peptide (measured by reference to incorporation of 3H thymidine);

FIG. 9 shows two-colour FACS analysis profiles of CD69 expression in CD3 positive T-cells, in CD4+ T-cells activated for 72 hours with anti-CD3 and anti-CD28 alone (activated only) or with Shh added at 100 ng/ml;

FIG. 10 shows two-colour FACS analysis profiles of CD25 expression in CD3 positive T-cells, in CD4+ T-cells activated for 72 hours with anti-CD3 and anti-CD28 alone (activated only) or with Shh added at 100 ng/ml;

FIG. 11 shows the effect of Shh on the survival, in vitro, of human CD4+ T-cells without any other stimulus. Survival was measured by Trypan blue staining of dead and alive cells in culture;

FIG. 12 shows the effect of Shh (10 ng/ml and 100 ng/ml) on mouse spleen cells and purified CD4+ T-cells in culture (no activation). Cells cultured in medium without Shh were used as controls;

FIG. 13 shows the effect of Shh (10 ng/ml and 500 ng/ml) on cell cycle progression in CD4+ T-cells cultured in a medium alone or with anti-CD3 and anti-CD28 antibodies and with or without anti-Shh neutralising mAb;

FIG. 14 shows Taqman real time PCR analysis of Shh, Ihh, HIP and Ptc expression in CD4+ T-cells in the presence and absence of anti-CD3/CD28 antibodies and/or in the presence and absence of Shh.

FIG. 15 shows real time PCR analysis of Shh, Ihh, HIP and Ptc expression in CD4+ T-cells in the presence of CD3/CD28, with or without IFN-g cytokine;

FIG. 16 shows Shh induced T-cell survival requires Gli signalling. Cell survival was measured in Gli2⁻/Gli3⁻ mice cells (i.e. from mice in which one allele each of Gli2 and Gli3 were deleted from the genome) in the presence (10 ng/ml and 500 ng/ml) and absence of Shh;

FIG. 17 shows expression analysis of Shh, Ihh, Ptc and Hip in CD4+ T-cells in the presence of α-CD3/CD28. Expression in medium, in the presence of 50 ng/ml IL-10 and in the presence of 50 ng/ml IFN-γ was compared to expression in resting CD4+ T-cells;

FIG. 18 shows the expression of components of the Hh signalling pathway in peripheral CD4+ cells. FIG. 18(A) shows an RT-PCR analysis of the expression of shh (S), Gli1 (G), smo (Sm) and ptc (P) in adult mouse normal thymus (a; as positive control), activated CD4⁺ T-cells (b) and resting CD4⁺ T-cells (c), with size of product given (bp=base pairs). Water (H₂O) was used as a negative control. FIGS. 18(B-D) show immunocytochemistry analysis of expression of Shh (C) and ptc (D) protein in the spleen. An isotype control is also shown (B). Original magnification ×400.

FIG. 19 shows that exogenous Shh increases proliferation of sub-optimally activated CD4+ T-cells. FIG. 19(A) shows proliferation of resting T-cells in medium alone (open bars) or cultured with Shh peptide (500 ng/ml; shaded bars); CD4⁺ T-cells optimally activated with anti-CD3 (1.0 μg/ml) and anti-CD28 (5 μgs/ml) antibodies alone (open) or with Shh added (filled) at 0 or 24 hr before activation. FIG. 19(B) shows proliferation of CD4⁺ T-cells sub-optimally activated with anti-CD3 (0.25 μg/ml) and anti-CD28 (0.1 μg/ml) antibodies alone (open) or in the presence of 500 ng/ml Shh peptide (filled) added at time 0 or 24 hr before activation. Data given are mean cpm counts from 3 separate experiments. * Significantly higher than proliferation in absence of Shh p=<0.01, ** p=<0.04.

FIG. 20 shows that Shh promotes entry of activated but not resting CD4+ T-cells into S/G2 phase. FIGS. 20(A and B) shows representative plots of cell cycle analysis of CD4⁺ T-cells activated with anti-CD3 (1 μg/ml) and anti CD28 (5 μg/ml) in the absence (A) or presence of Shh (B). FIG. 20(C) shows data from representative cell cycle analysis of resting CD4⁺ T-cells in the absence or presence of Shh peptide (500 ng/ml) at 24, 48 or 72 hr. FIG. 20(D) shows data from a representative cell cycle analysis of CD4⁺ T-cells optimally activated by anti CD3 (1 μg/ml) and anti CD28 (5 μg/ml) antibodies or sub-optimally activated by anti CD3 (0.25 μg/ml) and anti CD28 (1 μg/ml) antibodies in the absence of Shh or in the presence of 500 ng/ml Shh peptide added at time 0 or 24 hr before activation.

FIG. 21 shows that neutralising anti-Shh antibody inhibits TCR mediated CD4+ T-cell proliferation. CD4⁺ T-cell proliferation was measured by ³H-TdR incorporation and determined at 72 hr. CD4⁺ T-cells were sub-optimally activated with anti-CD3 and anti-CD28 antibodies and the neutralising anti-Shh antibody (5E1) or isotype control added at the time of activation.

FIG. 22 shows a kinetic analysis of Shh, ptc, Gli1 and bcl-2 gene expression in activated CD4⁺ T-cells with and without exogenous Shh. CD4⁺ T-cells were activated with sub-optimal concentrations of anti-CD3 and anti-CD28 in medium alone (A) or with exogenous Shh added at the time of activation (B). Cells were collected at 24, 48 and 72 hr and RNA isolated for the measurement of transcripts by real time PCR.

FIG. 23 shows the relative level of Shh and bcl-2 gene expression in activated CD4⁺ T-cells with and without exogenous Shh. CD4⁺ T-cells were activated with sub-optimal concentrations of anti-CD3 and anti-CD28 in medium alone or with exogenous Shh added at the time of activation. Cells were collected at 24, 48 and 72 hr and RNA isolated for the measurement of transcripts by real time PCR. In order to examine the effect of exogenous Shh peptide on the transcription of Shh (A) and bcl-2 (B), the RNA samples from activated CD4⁺ T-cell cultures with addition of Shh peptide were normalised against the media only activated cultures at equivalent time points.

FIG. 24 shows that neutralising anti-Shh antibody inhibits CD4+ T-cell entry into S/G2 phase of the cell cycle. In more detail, it shows a cell-cycle analysis of a representative experiment where CD4⁺ T-cells were sub-optimally activated with anti-CD3 (0.25 μg/ml) ant anti-CD28 (0.1 μg/ml) antibodies in the absence or presence of neutralising anti-Shh antibody (5E1).

### DETAILED DESCRIPTION

For ease of reference a summary of the accompanying sequence listings is given below:

SEQ ID NO:1 shows the deduced amino acid sequence of mouse SHH and SEQ ID NO:2 shows the corresponding nucleic acid sequence;

SEQ ID NO:3 shows the deduced amino acid sequence of mouse Dvl-1 and SEQ ID NO:4 shows the corresponding nucleic acid sequence;

SEQ ID NO:5 shows the deduced amino acid sequence of mouse HIP and SEQ ID NO:6 shows the corresponding nucleic acid sequence; and

SEQ ID NO:7 shows the deduced amino acid sequence of mouse WIF-1 and SEQ ID NO:8 shows the corresponding nucleic acid sequence.

Hedgehog Family Proteins

All multicellular organisms require cell communication to regulate growth and differentiation in the embryo. One strategy for this is to establish discrete organising centres that emit signals to coordinately control cell proliferation and cell fate determination. The hedgehog (hh) gene was identified originally through the segment polarity phenotype caused by its mutation in Drosophila. Genes of the hh family have now been isolated from several vertebrate species, including mouse, chicken, zebrafish, rat, Xenopus and human. The genes not only seem to show a high degree of structural homology both within and between species, but in addition exhibit some remarkable similarities in the ways in which they function in various embryonic processes. In vertebrates, Sonic hedgehog (Shh) is a key signal in several signalling centres. There are two other mammalian HH members, Indian hedgehog (Ihh) and Desert hedgehog (Dhh).

A summary of various hedgehog genes is given in the following Table 1:

TABLE 1

| Gene | Species |
|---|---|
| hedgehog (hh) | Drosophila |
| Sonic hedgehog (Shh) | Mouse, Human, Rat, Xenopus, |
| | Chicken, Zebrafish |
| Indian hedgehog (Ihh) | Mouse, Human, Chicken |
| Desert hedgehog (Dhh) | Mouse |
| Banded hedgehog (X-bhh) | Xenopus |
| Cepalic hedgehog (X-chh) | Xenopus |
| tiggy-winkle hedgehog (twhh) | Zebrafish |
| echidna hedgehog (ehh) | Zebrafish |

The classification of genes from different species is based on the comparison of the expression pattern and the amino acid sequence. Of all vertebrate proteins, DHH is most similar to Drosophila HH (51% identity over entire length of processed proteins). Amino acid identity among SHH is 93% between mouse and human, 84% between mouse and chicken, 78% between mouse and Xenopus, and 68% between mouse and zebrafish. Intraspecies comparison within the mouse reveals 58-63% identity in pairwise combination between SHH, IHH and DHH. Interspecies comparison between the mouse and Xenopus reveals highest identities between IHH and XBHH (70%) and DHH and XCHH (64%).

The various Hedgehog proteins consist of a signal peptide, with a highly conserved N-terminal region and a more divergent C-terminal domain. It is understood that the biologically active Hedgehog peptides are formed from a larger precursor protein. In addition to signal sequence cleavage in the secretory pathway, Hedgehog precursor proteins undergo an internal autoproteolytic cleavage. This autocleavage generates an N-terminal peptide (about 19 kDa) and a C-terminal peptide (of about 26-28 kDa). It is this N-terminal peptide that is necessary for short- and long-range Hedgehog signalling activities in Drosophila and vertebrates. The N-terminal peptide stays tightly associated with the surface of cells in which it is synthesised, while the C-terminal peptide is freely diffusable.

Signalling Pathway

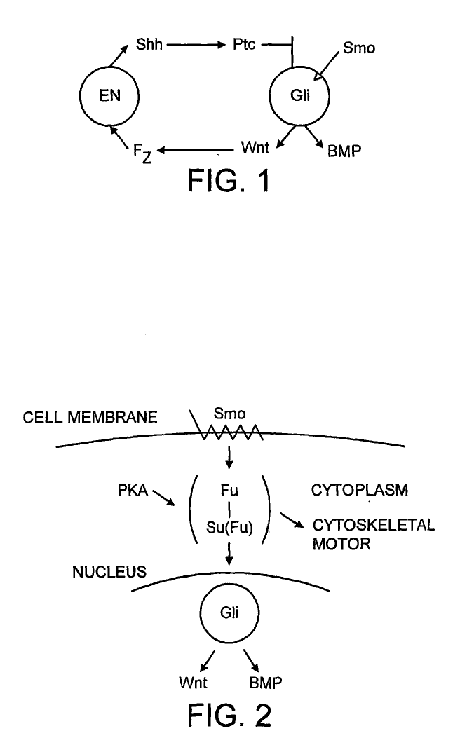

FIG. 1 shows one representation of a Hedgehog signalling pathway, with particular reference to signalling in vertebrates.

Epithelial cells may express the homeodomain transcription factor engrailed (en) and secrete Hedgehog protein shown for illustrative purposes in the Figure as Shh. We have observed that En plays an important role in the maintenance of lymphocyte survival in the peripheral immune system.

In targeT-cells, HH signalling is mediated by two transmembrane proteins patched (Ptc) which has structural similarities to channel and transporter proteins, and Smoothened (Smo), a seven-transmembrane protein similar to G-protein coupled receptors and the Wingless receptor Frizzeled (described below). Smo is a constitutive activator of HH target genes. Its activity is normally repressed by Ptc, and this repression is relieved by HH binding to Ptc. Thus, binding of HH to Ptc allows signal transduction leading to activation of the transcription factor Gli, which is located in the nucleus of the targeT-cells.

The signal reaches Gli through the cytoplasmic complex formed between (1) the serine/threonine kinase Fused (Fu), (2) Suppressor of Fused (SU(Fu)); and (3) Costal2 (Cos2). Signalling through this complex may be inhibited by the cAMP-dependent protein kinase A (PKA) (see FIG. 2).

Gli acts on target genes wingless (Wnt) and the BMP/activin growth factors. Both Wnt and BMP are secreted to the extracellular fluid to bind to their receptors. This process is illustrated schematically in FIG. 1.

A summary and comparison of components of the Hedgehog signalling pathway is given below in Table 2:

TABLE 2

| Drosophila | Vertebrate |
|---|---|
| En | En 1, 2 |
| Hh | Ihh, Dhh, Shh |
| Ptc | Ptc 1, 2 |
| Smo | Smo |
| Ci | Gli 1-3 |
| Target genes | |
| Wg | Wnt˜15 |
| Dpp ≡ TGF_{β} | BMP 8-10 |

Vertebrate and non-vertebrate nomenclature may be used interchangeably herein.

Shh, ptc & smo transcripts are present in primitive and mature CD19⁺, CD33⁺ and CD3⁺ cell populations. Members of the Shh signalling pathway regulate differentiation of T-cells from the double negative (CD4⁻CD8⁻) to the double positive (CD4⁺CD8⁺) stage of T-cell development.

Shh has a proliferative effect on a variety of cell types including hematopoietic stem cells (Fan and Khavari; Bhardwaj et al; Fujita et al; Kenny and Rowitch; and Outram et al). Shh and ptc protein are expressed in peripheral lymphoid tissue. The Shh signalling pathway components Shh, ptc, smo and Gli1 are present in both resting and activated peripheral CD4⁺ T-cells. It has been demonstrated that members of the Shh signalling pathway are expressed in the thymus (Outram, et al.). Shh is present in thymic epithelial cells but not thymocytes. By contrast, the receptors smo and ptc have been detected in thymocytes at various stages of development (Outram, et al.). Furthermore, transcripts for Shh, ptc and smo have been detected in mature CD3⁺ T-cell populations (Bhardwaj, et al.).

Shh is thought to function as a cofactor and contribute to clonal expansion of T-cells under physiological conditions of stimulation. Shh increases proliferation in activated CD4⁺ T-cells. It appears to promote CD4⁺ T-cell entry into the proliferative S/G2 phase of the cell cycle. This effect of Shh has been reported for several other cell types (Fan and Khavari; Kenny and Rowitch). For example, it has been demonstrated that Shh induced a disproportionate number of keratinocytes in S/G2 phase of the cell cycle (Fan and Khavari). Kenney & Rowitch found that Shh increased the number of neuronal precursor cells in S phase.

Shh does significantly increase the transcription of bcl-2. Shh has previously been shown to induce expression of bcl-2 (Fan et al). Bcl-2 is known to play an important role in the regulation of post-thymic T-cell survival (Strasser et al; Katsuma et al; Nakayama et al; and Veis et al). Thus, Shh is thought to act, at least in part, by promoting survival of activated cells through the induction of bcl-2.

Cell cycle progression is largely dependent on a regulatory network whose key components include the cyclins and cyclin-dependent kinases (cdks) (Lees; Morgan; Sherr; Sherr; and Elledge). It has previously been shown that Shh expression is associated with increased activity of cdk2 & 4, important in G1 to S transition, in keratinocytes under normal growth conditions (Fan and Khavari). It has also been shown that Shh promotes cell cycle progression in proliferating neuronal precursors by maintaining expression of G1 phase cyclins such as cyclin D1, D2 & E, thought to be via synthesis of unknown protein intermediates (Kenny and Rowitch).

Entry into mitosis requires the activation and nuclear translocation of the M phase promoting factor (MPF) (Borgne et al; and Peter et al). The MPF consists of 2 proteins—cdc2 and cyclin B1. Patched 1 can interact with cyclin B1 and prevent nuclear translocation of the MPF and thereby prevenT-cell cycle progression. With addition of Shh to bind ptc, the release of cyclin B1 is facilitated, and nuclear import of the MPF and subsequently cell cycle progression can take place (Barnes et al). However, the effects of Shh on the cell cycle in CD4⁺ T-cells occurred in S phase, which implies that repression of MPF (which controls the latter G2/M phase) is not the sole factor involved. The repressive effect of ptc on cell cycle progression could explain why the transcription of ptc mRNA does not increase throughout the course of proliferation as in the case of Shh mRNA and Gli1 mRNA.

In summary, Shh signalling plays an important role in sustained and enhanced peripheral CD4⁺ T-cell proliferation. This may occur via promotion of CD4⁺ T-cells into S/G2 phase of the cell cycle. Furthermore, Shh can be produced in an autocrine fashion by the CD4⁺ T-cells themselves, functioning to amplify and maintain clonal expansion.

Further information on Hedgehog signalling may be find in the following articles: Ingham; Chuang and McMahon; Pepicelli et al; Hammerschmidt et al; Bhardwaj et al; and Outram et al.

Wingless/Wnt Signalling Pathway

We have examined the role for dysregulation of the Wnt signalling pathway in interstitial lung disease. The Wnt genes are targets of the HH pathway, and the Wnt proteins are secreted growth factors which are involved in the regulation of epithelial cell proliferation and differentiation in the lung during embryonic development. We propose that Wnt signalling may also be upregulated during processes of epithelial cell repair in the lung.

Dishevelled-1 (Dvl-1) is the murine homolog of the fly Dsh gene and functions to transmit signals from the Wnt receptor, Frizzled, to the cytoplasm, where it regulates the kinase activity of a well known serine/threonine kinase, GSK-3b. Over expression of Dsh in fly epithelia leads to oncogenic activation of the epithelium by increasing Wnt signalling.

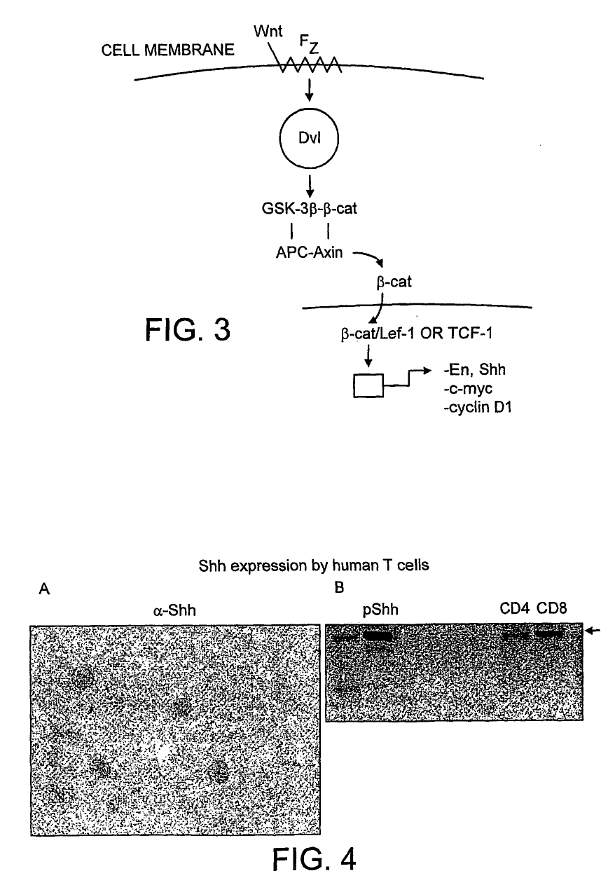

A representation of this pathway is shown in FIG. 3. Wingless (Wg), in Drosophila, and, its vertebrate homolog, Wnt signalling pathways regulate cell profileration. Wg and Wnt are secreted growth factors which are involved in triggering cellular decisions. The Wg/Wnt ligand binds to Frizzled (Fz) family receptor molecules to initiate a signal transduction cascade involving the cytoplasmic protein Dishevelled (Dvl) (Sussman D J et al). The GenBank accession number for Dvl-1 cDNA is U10115. The complex illustrated in FIG. 3 is present in the cytoplasm of the targeT-cell. Generally APC blocks signalling; however, in the presence of signalling from Wnt, β-catenin is released and interacts with two transcription factors—Lef-1/TCF-1 resulting in target gene expression. Target genes of Wnt include En and therefore indirectly HH, c-myc and cyclin D1.

Modulators

The present invention relates to the use of compounds which inhibit or block (antagonise) Hedgehog signalling. Such compounds may be seen as having the effect of downregulating the expression of Hedgehog. Similarly the present invention also relates to the use of compounds which inhibit or block (antagonise) a signalling pathway which is a target of the Hedgehog signalling pathway. Conveniently such compounds may be referred to herein as inhibitors or antagonists.

The present invention also relates to the use of compounds which increase (agonise) Hedgehog signalling. Similarly the present invention also relates to the use of compounds which increase (agonise) a signalling pathway which is a target of the Hedgehog signalling pathway. Conveniently such compounds may be referred to as upregulators or agonists.

The invention contemplates that mutations that result in loss of normal function of the regulators of the Hedgehog signalling pathway or regulators of a pathway which is a target of the Hedgehog signalling pathway in human disease states in which lymphocyte infiltration or failure of a cell cycle checkpoint is involved. Gene therapy to restore such regulatory activity would thus be indicated in treating those disease states Alternatively, it is contemplated that preventing the expression of or inhibiting the activity of such signalling pathways will be useful in treating the disease states. It is contemplated that antisense therapy or gene therapy could be applied to negatively regulate such signalling pathways.

Antagonists for each component of the signalling pathway have been identified. These may be summarised as follows in Table 3:

TABLE 3

| Component | Antagonist |
|---|---|
| HH | Hip (Chuang and McMahon), Veratrum |
| | alkaloids and distal inhibitors of |
| | cholesterol biosynthesis (Cooper et |
| | al) e.g. cyclopamine (Coventry et al). |
| Wnt | Frzb (Leyns et al), Cerberus (Bouwmeester |
| | et al), Gremlin (Hsu et al), |
| | WIF-1 (Hsieh et al) |
| Activin | Follistatin (Iemura et al) |

HIP (for Hedgehog-interacting protein) is a membrane glycoprotein that binds to at least all three mammalian Hedgehog proteins with an affinity comparable to that of Ptc. HIP appears to attenuate Hedgehog signalling as a result of binding to Hedgehog proteins. Such a negative regulatory feedback loop could also serve to modulate the response to any Hedgehog signal. The GenBank accession number for HIP is AF116865.

Veratrum alkaloids and distal inhibitors of cholesterol biosynthesis have been studied for more than 30 years as potent teratogens capable of inducing cyclopia and other birth defects. It has also been shown that these compounds specifically block the Shh signaling pathway (Cooper et al). One example of such a veraturm alkaloid is cyclopamine (11-deoxojervine), a steroid isolated from the desert plant Veratrum californicum (Coventry et al).

Frzb (Frezzled) is a secreted antagonist of Wnt signalling. Frzb contains a domain similar to the putative Wnt-binding region of the Frizzled family of transmembrane receptors, but it lacks all the transmembrane domains resulting in a putative secreted Wnt-binding protein. The GenBank accession numbers for the Xenopus, mouse and human Frzb cDNA sequences are U68059, U68058 and U68057, respectively.

Cerberus is a secreted protein and it has been found to be an antagonist of the Wnt signalling pathway. The GenBank accession number for the Xenopus Cerberus cDNA is U64831.

WIF-1 (Wnt-inhibitory factor-1) is a secreted protein which binds to Wnt proteins and inhibits their activities. GenBank accession numbers for WIF-1 are: human, AF122922; mouse, AF122923; Xenopus, AF122924; and zebrafish, AF122925.

Gremlin is a secreted protein and it has been found to be an antagonist of the Wnt signalling pathway. The GenBank accession numbers for Gremlin cDNA are: Xenopus, AF045798; chick, AF045799; human, AF045800; and mouse, AF045801.

It will also be appreciated that the antagonist or agonist may itself be a component of the Hedgehog signalling pathway, or a component of the target pathway of the Hedgehog signalling pathway. Examples of such antagonists include the negative regulators of HH signalling: Ptc, Cos2 and PKA. Examples of such agonists include the positive regulators of HH signalling Smo and Gli.

In a particularly preferred embodiment use is made of PKA. PKA has been implicated in the mechanism of Hh signal transduction because it acts to repress Hh target genes in imaginal disc cells that express Ci. Ci action as transcriptional repressor or activator is contingent upon Hedgehog-regulated, PKA-dependent proteolytic processing.

Cyclic AMP (cAMP) is a nucleotide that is generated from ATP in response to hormonal stimulation of cell-surface receptors. cAMP acts as a signaling molecule by activating A-kinase; it is hydrolyzed to AMP by phosphodiesterase (PDE). cAMP levels affect cubitus cleavage and TGF-β levels. Specifically, when cAMP levels increase, TGF-β levels decrease. In another embodiment of the invention use is made of cAMP modifiers in treatment. Such modifiers include PDE inhibitors, and beta-agonists such as the beta-adrenergic agonist. For example, it has been found that ptc 1 transcription can be induced by agents activating the cAMP signal transduction pathway. Agents which elevate intracellular cAMP levels are well known in the art and we have shown that such agents could be used in the present invention throuhg their reduction of TGF-beta production.

Immunosuppressive cytokines may also be used to modulate the Hedgehog signalling pathway. Examples include members of the TGF-β family such as TGF-β-1 and TGF-β-2, and interleukins such as IL-4, IL-10 and IL-1 3, IFN-γ, and FLT3 ligand.

Antisense nucleic acids (preferably 10 to 20 base pair oligonucleotides) capable of specifically binding to expression control sequences or RNA are introduced into cells (e.g., by a viral vector or colloidal dispersion system such as a liposome). The antisense nucleic acid binds to the target sequence in the cell and prevents transcription or translation of the target sequence. Phosphothioate and methylphosphate antisense oligonucleotides are specifically contemplated for therapeutic use by the invention. The antisense oligonucleotides may be further modified by poly-L-lysine, transferrin polylysine, or cholesterol moieties at their 5′ end.

Also comprehended by the present invention are antibody products (e.g., monoclonal and polyclonal antibodies, single chain antibodies, chimeric antibodies, CDR-grafted antibodies and antigen-binding fragments thereof) and other binding proteins (such as those identified in the assays above). Binding proteins can be developed using isolated natural or recombinant enzymes. The binding proteins are useful, in turn, for purifying recombinant and naturally occurring enzymes and identifying cells producing such enzymes. Assays for the detection and quantification of proteins in cells and in fluids may involve a single antibody substance or multiple antibody substances in a “sandwich” assay format to determine cytological analysis of HH protein levels. The binding proteins are also manifestly useful in modulating (i.e. blocking, inhibiting, or stimulating) interactions.

Antibodies may be generated by administering polypeptides or epitope-containing fragments to an animal, usually a rabbit, using routine protocols. Examples of such techniques include those in Kohler and Milstein.

In more detail in one embodiment, the modulator of hedgehog signalling may be, for example, a genetically engineered soluble fusion protein comprising a hedgehog protein or polypeptide, or a fragment thereof, and any of various portions of the constant regions of heavy or light chains of immunoglobulins of various subclasses (eg IgG, IgM, IgA, IgE). Preferred as an immunoglobulin component of such a fusion protein is the constant part of the heavy chain of human IgG, particularly IgG1, where fusion takes place at the hinge region. In a particular embodiment, the Fc part can be removed simply by incorporation of a cleavage sequence which can be cleaved with blood clotting factor Xa. Examples of fusion protein technology can be found in International Patent Application Nos. WO94/29458 and WO94/22914.

More generally, the modulators may be selected from polypeptides and fragments thereof, linear peptides, cyclic peptides, synthetic and natural compounds including low molecular weight organic or inorganic compounds. The modulator may be derived from a biological material such as a component of extracellular matrix.

Polypeptide substances may be purified from mammalian cells, obtained by recombinant expression in suitable hosT-cells or obtained commercially. Alternatively, nucleic acid constructs encoding the polypeptides may be introduced by transfection using standard techniques or viral infection/transduction.

Modulators for use according to the present invention may be conveniently identified using a convenient screening procedure.

One assay for identifying such modulators may involve immobilizing a component of the relevant pathway, e.g. HH, or a test protein, detectably labelling the nonimmobilized binding partner, incubating the binding partners together and determining the amount of label bound. Bound label indicates that the test protein interacts with the component.

Another type of assay for identifying modulators involves immobilizing a component of the pathway, e.g. HH, or a fragment thereof on a solid support coated (or impregnated with) a fluorescent agent, labelling a test protein with a compound capable of exciting the fluorescent agent, contacting the immobilized component with the labelled test protein, detecting light emission by the fluorescent agent, and identifying interacting proteins as test proteins which result in the emission of light by the fluorescent agent. Alternatively, the putative interacting protein may be immobilized and the component may be labelled in the assay.

Moreover, such assays for identifying modulators may involve: transforming or transfecting appropriate hosT-cells with a DNA construct comprising a reporter gene under the control of a promoter regulated by a transcription factor having a DNA-binding domain and an activating domain; expressing in the hosT-cells a first hybrid DNA sequence encoding a first fusion of part or all of a component of the pathway, e.g. HH or Wnt, and the DNA binding domain or the activating domain of the transcription factor; expressing in the hosT-cells a second hybrid DNA sequence encoding part or all of a protein that interacts with said component and the DNA binding domain or activating domain of the transcription factor which is not incorporated in the first fusion; evaluating the effect of a test compound on the interaction between said component and the interacting protein by detecting binding of the interacting protein to said component in a particular hosT-cell by measuring the production of reporter gene product in the hosT-cell in the presence or absence of the test compound; and identifying modulating compounds as those test compounds altering production of the reported gene product in comparison to production of the reporter gene product in the absence of the modulating compound. Presently preferred for use in the assay are a lexA promoter to drive expression of the reporter gene, the lacZ reporter gene, a transcription factor comprising the lexA DNA binding domain and the GALA transactivation domain, and yeast hosT-cells.

In a particular embodiment described in relation to Hedgehog signalling the appropriate hosT-cell is transformed or transfected with a DNA construct comprising a reporter gene under the control of the Ptc promoter; expressing in said cells a DNA sequence encoding Hedgehog; evaluating the effect of a test compound on the interaction between HH and the Ptc promoter in a particular hosT-cell by measuring the production of reporter gene product in the hosT-cell in the absence and presence of the test compound; and identifying modulators as those test compounds reducing the production of the reporter gene product in comparison to production of the reporter gene product in the absence of the test compound.

Analogous assays may be used for modulators of the target pathways of Hedgehog signalling. For example, for the Wnt signalling pathway, the ability of a compound to modulate the interaction of Wnt and Fz may be determined.

Combinatorial libraries, peptide and peptide mimetics, defined chemical entities, oligonucleotides, and natural product libraries may be screened for activity as modulators in such assays.

The present invention also relates to the use of derivatives, variants, fragments, analogues, homologues and mimetics of the modulators mentioned above, including those identifiable using the assay procedures. Assays in accordance with the present invention are described in more detail below.

The term “derivative” as used herein in relation to the polypeptides of the present invention includes any substitution of, variation of, modification of, replacement of, deletion of, or addition of one (or more) amino acid residues from or to the sequence providing that the resultant protein etc., possesses the capability to agonise or antagonise the action of the signalling pathway.

The term “variant” as used herein in relation to the polypeptides of the present invention includes any substitution of, variation of, modification of, replacement of, deletion of, or addition of one (or more) amino acid residues from or to the sequence providing that the resultant protein etc., possesses the capability to agonise or antagonise the action of the signalling pathway.

The term “fragment” as used herein in relation to the polypeptides of the present invention includes a variant polypeptide which has an amino acid sequence that is entirely the same as part but not all of the amino acid sequence of the aforementioned polypeptide and possesses the capability to agonise or antagonise the action of the signalling pathway.

The term “analogue” as used herein in relation to the polypeptides of the present invention includes any peptidomimetic, i.e. a chemical compound that possess the capability to agonise or antagonise the action of the signalling pathway in a similar manner to the parent polypeptide.

The term “homologue” as used herein in relation to the polypeptides of the present invention includes a polypeptide which has the same evolutionary origin as the subject polypeptide providing that it possesses the capability to agonise or antagonise the action of the signalling pathway.

The term “mimetic” as used herein in relation to the inhibitors of the present invention includes a compound which also possesses the capability to agonise or antagonise the action of the signalling pathway in a similar manner to the parent compound.

More particularly, the term “homologue” covers identity with respect to structure and/or function providing the expression product of the resultant nucleotide sequence has the inhibitory or upregulatory activity. With respect to sequence identity (i.e. similarity), preferably there is at least 75%, more preferably at least 85%, more preferably at least 90% sequence identity. More preferably there is at least 95%, more preferably at least 98%, sequence identity. These terms also encompass allelic variations of the sequences.

Sequence identity with respect to the sequences can be determined by a simple “eyeball” comparison (i.e. a strict comparison) of any one or more of the sequences with another sequence to see if that other sequence has, for example, at least 75% sequence identity to the sequence(s).

Relative sequence identity can also be determined by commercially available computer programs that can calculate % identity between two or more sequences using any suitable algorithm for determining identity, using for example default parameters. A typical example of such a computer program is CLUSTAL. Advantageously, the BLAST algorithm is employed, with parameters set to default values. The BLAST algorithm is described in detail at http://www.ncbi.nih.gov/BLAST/blast-help.html, which is incorporated herein by reference. The search parameters are defined as follows, can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see http://www.ncbi.nih.gov/BLAST/blast_help.html) with a few enhancements. The BLAST programs were tailored for sequence similarity searching, for example to identify homologues to a query sequence. For a discussion of basic issues in similarity searching of sequence databases, see Altschul et al (1994) Nature Genetics 6:119-129.

The five BLAST programs available at http://www.ncbi.nlm.nih.gov perform the following tasks:

blastp—compares an amino acid query sequence against a protein sequence database.

blastn—compares a nucleotide query sequence against a nucleotide sequence database.

blastx—compares the six-frame conceptual translation products of a nucleotide query sequence (both strands) against a protein sequence database.

tblastn—compares a protein query sequence against a nucleotide sequence database dynamically translated in all six reading frames (both strands).

tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST.

Other computer program methods to determine identify and similarity between the two sequences include but are not limited to the GCG program package (Devereux et al 1984 Nucleic Acids Research 12: 387) and FASTA (Atschul et al 1990 J Molec Biol 403-410).

In some aspects of the present invention, no gap penalties are used when determining sequence identity.

As used herein the terms protein and polypeptide and peptide may be assumed to be synonymous, protein merely being used in a general sense to indicate a relatively longer amino acid sequence than that present in a polypeptide, and polypetide merely being used in a general sense to indicate a relatively longer amino acid sequence than that present in a peptide. Generally for ease of reference only we will simply refer to the term polypeptide.

The present invention also encompasses use of nucleotide sequences that are complementary to the sequences presented herein, or any fragment or derivative thereof. If the sequence is complementary to a fragment thereof then that sequence can be used as a probe to identify similar promoter sequences in other organisms.

The present invention also encompasses use of nucleotide sequences that are capable of hybridising to the sequences presented herein, or any fragment or derivative thereof.

Hybridization means a “process by which a strand of nucleic acid joins with a complementary strand through base pairing” (Coombs J (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Also included within the scope of the present invention are use of nucleotide sequences that are capable of hybridizing to the nucleotide sequences presented herein under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related nucleotide sequences.

In a preferred aspect, the present invention covers use of nucleotide sequences that can hybridise to the nucleotide sequences of the present invention under stringent conditions (e.g. 65° C. and 0.1×SSC).

The present invention also encompasses use of nucleotide sequences that are capable of hybridising to the sequences that are complementary to the sequences presented herein, or any fragment or derivative thereof. Likewise, the present invention encompasses use of nucleotide sequences that are complementary to sequences that are capable of hybridising to the sequence of the present invention. These types of nucleotide sequences are examples of variant nucleotide sequences.

In this respect, the term “variant” encompasses sequences that are complementary to sequences that are capable of hydridising to the nucleotide sequences presented herein. Preferably, however, the term “variant” encompasses sequences that are complementary to sequences that are capable of hydridising under stringent conditions (eg. 65° C. and 0.1×SSC {1×SSC=0.15 M NaCl, 0.015 Na₃ citrate pH 7.0}) to the nucleotide sequences presented herein.

In one embodiment, for example, the modulator of the hedgehog signalling pathway may be a hedgehog protein, either in substantially full length form or in the form of a bioactive fragment.

As reported in WO 0164238 (Curis) examples of bioactive fragments of hedgehog polypeptides are described, for example in PCT publications WO 95/18856 and WO 96/17924.

As reported in WO 0164238 there are a wide range of lipophilic moieties or groups with which such hedgehog polypeptides can, if desired, be derivatived. The term “lipophilic group”, in the context of being attached to a hedgehog polypeptide, refers to a group having high hydrocarbon content thereby giving the group high affinity to lipid phases. A lipophilic group can be, for example, a relatively long chain alkyl or cycloalkyl (preferably n-alkyl) group having approximately 7 to 30 carbons. The alkyl group may terminate with a hydroxy or primary amine “tail”. To further illustrate, lipophilic molecules include naturally-occurring and synthetic aromatic and non-aromatic moieties such as fatty acids, sterols, esters and alcohols, other lipid molecules, cage structures such as adamantane and buckminsterfullerenes, and aromatic hydrocarbons such as benzene, perylene, phenanthrene, anthracene, naphthalene, pyrene, chrysene, and naphthacene.

In one embodiment, a hedgehog polypeptide may be modified with one or more sterol moieties, such as cholesterol (see, for example, PCT publication WO 96/17924). In certain embodiments, the cholesterol is preferably added to the C-terminal glycine were the hedgehog polypeptide corresponds to the naturally-occurring N-terminal proteolytic fragment. In another embodiment, the hedgehog polypeptide can be modified with a fatty acid moiety, such as a myrostoyl, palmitoyl, stearyl, or arachidoyl moiety. (see, for example, Pepinsky et al. (1998) J Biol. Chem 273: 14037).

In addition to those effects seen by cholesterol-addition to the C-terminus or fatty acid addition to the N-terminus of extracellular fragments of the protein, at least certain of the biological activities of the hedgehog gene products are unexpectedly potentiated by derivativation of the protein with lipophilic moieties at other sites on the protein and/or by moieties other than cholesterol or fatty acids. Certain aspects of the invention are directed to the use of preparations of hedgehog polypeptides which are modified at sites other than N-terminal or C-terminal residues of the natural processed form of the protein, and/or which are modified at such terminal residues with lipophilic moieties other than a sterol at the C-terminus or fatty acid at the N-terminus.

Particularly useful as lipophilic molecules are alicyclic hydrocarbons, saturated and unsaturated fatty acids and other lipid and phospholipid moieties, waxes, cholesterol, isoprenoids, terpenes and polyalicyclic hydrocarbons including adamantane and buckminsterfullerenes, vitamins, polyethylene glycol or oligoethylene glycol, (C₁-C₁₈)-alkyl phosphate diesters, —O—CH₂—CH(OH)—O—(C₁₂-C₁₈)-alkyl, and in particular conjugates with pyrene derivatives. The lipophilic moiety can be a lipophilic dye suitable for use in the invention include, but are not limited to, diphenylhexatriene, Nile Red, N-phenyl-1naphthylamine, Prodan, Laurodan, Pyrene, Perylene, rhodamine, rhodamine B, tetramethylrhodamine, Texas Red, sulforhodamine, 1,1′-didodecyl-3,3,3′,3′-tetramethylindocarbocyanine perchlorate, octadecyl rhodamine B and the BODIPY dyes available from Molecular Probes Inc.

The hedgehog polypeptide or bioactive fragment may be linked to the hydrophobic moiety by any suitable means, including by chemical coupling means, or by genetic engineering.

WO 01/98344 (Amgen) also describes a number of protein, nucleic acid and antibody modulators of the hedgehog signalling pathway, in the form of both agonists and antagonists, for example as follows:

Production of Fragments and Analogs

As described in WO 01/98344, fragments of a hedgehog signalling protein can be produced efficiently by recombinant methods, by proteolytic digestion, or by chemical synthesis using methods known to those of skill in the art. In recombinant methods, internal or terminal fragments of a polypeptide can be generated by removing one or more nucleotides from one end (for a terminal fragment) or both ends (for an internal fragment) of a DNA sequence which encodes for the isolated hedgehog polypeptide. Expression of the mutagenized DNA produces polypeptide fragments. Digestion with “end nibbling” endonucleases can also be used to generate DNAs which encode an array of fragments. DNAs which encode fragments of a protein can, for example, be generated by random shearing, restriction digestion, or a combination of both. Protein fragments can be generated directly from intact proteins. Peptides can be cleaved specifically by proteolytic enzymes, including, but not limited to plasmin, thrombin, trypsin, chymotrypsin, or pepsin. Each of these enzymes is specific for the type of peptide bond it attacks. Trypsin catalyzes the hydrolysis of peptide bonds in which the carbonyl group is from a basic amino acid, usually arginine or lysine. Pepsin and chymotrypsin catalyse the hydrolysis of peptide bonds from aromatic amino acids, such as tryptophan, tyrosine, and phenylalanine. Alternative sets of cleaved protein fragments are generated by preventing cleavage at a site which is susceptible to a proteolytic enzyme. For instance, reaction of the E-amino acid group of lysine with ethyltrifluorothioacetate in mildly basic solution yields blocked amino acid residues whose adjacent peptide bond is no longer susceptible to hydrolysis by trypsin. Proteins can be modified to create peptide linkages that are susceptible to proteolytic enzymes.

For instance, alkylation of cysteine residues with 3-haloethylamines yields peptide linkages that are hydrolyzed by trypsin (Lindley, (1956) Nature 178,647). In addition, chemical reagents that cleave peptide chains at specific residues can be used. For example, cyanogen bromide cleaves peptides at methionine residues (Gross and Witkip, (1961) J. Am. Chem. Soc. 83, 1510). Thus, by treating proteins with various combinations of modifiers, proteolytic enzymes and/or chemical reagents, the proteins may be divided into fragments of a desired length with no overlap of the fragments, or divided into overlapping fragments of a desired length.

Fragments can also be synthesized chemically using techniques known in the art such as the Merrifield solid phase F-moc or t-Boc chemistry (eg Merrifield, Recent Progress in Hormone Research 23: 451 (1967)).

Production of Altered DNA and Peptide Sequences: Random Methods

As described in WO 01/98344, amino acid sequence variants of a protein can, for example, be prepared by random mutagenesis of DNA which encodes the protein or a particular portion thereof. Useful methods include PCR mutagenesis and saturation mutagenesis. A library of random amino acid sequence variants can also be generated by the synthesis of a set of degenerate oligonucleotide sequences. Examples of such methods include, for example: PCR Mutagenesis: (see, for example Leung et al., (1989) Technique 1,11-15); Saturation Mutagenesis: One method is described generally in Mayers et al., (1989) Science 229-242; and Degenerate Oligonucleotide Mutagenesis : (see for example Harang, S. A., (1983) Tetrahedron 39, 3; Itakura et al., (1984) Ann. Rev. Biochem. 53, 323 and Itakura et al., Recombinant DNA, Proc. 3rd Cleveland Symposium on Macromolecules, pp. 273-289 (A. G. Walton, ed.), Elsevier, Amsterdam, 1981.

Production of Altered DNA and Peptide Sequences: Directed Methods

Non-random, or directed, mutagenesis provides specific sequences or mutations in specific portions of a polynucleotide sequence that encodes an isolated polypeptide, to provide variants which include deletions, insertions, or substitutions of residues of the known amino acid sequence of the isolated polypeptide. The mutation sites may be modified individually or in series, for instance by: (1) substituting first with conserved amino acids and then with more radical choices depending on the results achieved; (2) deleting the target residue; or (3) inserting residues of the same or a different class adjacent to the located site, or combinations of options 1-3.

Such site-directed methods provide one way in which an N-terminal cysteine (or a functional equivalent) can be introduced into a given polypeptide sequence to provide the attachment site for a hydrophobic moiety. Suitable techniques include, for example:

Alanine scanning Mutagenesis: (see Cunningham and Wells, (1989) Science 244, 1081-1085);

Oligonucleotide-Mediated Mutagenesis: (see, for example, Adelman et al., (1983) DNA 2,183);

Cassette Mutagenesis: (see, for example, Wells et al., (1985) Gene 34, 315); and

Combinatorial Mutagenesis: (see, for example, Ladner et al., WO 88/06630).

Other Variants of Isolated Polypeptides

As described in WO 01/98344, hedgehog proteins can be generated to include a moiety, other than sequence naturally associated with the protein, that binds a component of the extracellular matrix and enhances localization of the analog to cell surfaces. For example, sequences derived from the fibronectin “type-III repeat”, such as a tetrapeptide sequence R-G-D-S (Pierschbacher et al. (1984) Nature 309: 30-3; and Komblihtt et al. (1985) EMBO 4: 1755-9) can be added to the hedgehog polypeptide to support attachment of the chimeric molecule to a cell through binding ECM components (Ruoslahti et al. (1987) Science 238: 491-497; Pierschbacheret al. (1987) J Biol. Chem. 262: 17294-8.; Hynes (1987) Cell 48: 549-54; and Hynes (1992) Cell 69: 11-25).

N-Modified Hedgehog Polypeptides as Antagonists

Certain hedgehog variants that contain N-terminal modifications can block hedgehog function because they lack the ability to elicit a hedgehog-dependent response but retain the ability to bind to hedgehog receptor, patched-1. For example, it has been reported that hedgehog polypeptides which either lack the N-terminal cysteine completely or contain this N-terminal cysteine in a modified form (e. g. chemically modified or included as part of an N terminal extension moiety), can act as hedgehog antagonists. Examples of hedgehog protein antagonists with such N-terminal modifications are included below:

N-Terminal Extensions

Antagonist polypeptides suitable for use in the invention may include a hedgehog polypeptide sequence in which the N-terminal cysteine is linked to an N-terminal extension moiety.

The isolated antagonist polypeptide can therefore be, for example, a recombinant fusion protein having: (a) a first N-terminal polypeptide portion that can be 5′ to the hedgehog polypeptide itself, and that contains at least one element (e. g., an amino acid residue) that may be unrelated to hedgehog, linked to (b) an N-terminal cysteine corresponding to Cys-1 of Sonic hedgehog that is part of a hedgehog antagonist of the invention, or a portion of hedgehog antagonist. This N-terminal extension moiety (e. g., the first N-terminal polypeptide portion) can be, for example, a histidine tag, a maltose binding protein, glutathione-S-transferase, a DNA binding domain, or a polymerase activating domain. The functional antagonist may include an N-terminal extension moiety that contains an element which replaces the Cys-1 of mature hedgehog or an N-terminal cysteine that corresponds to Cys-1 of a mature Sonic hedgehog.

N-Terminal Deletions

Another example of a functional antagonist is a hedgehog protein that is missing no greater than about 12 amino acids beginning from that N-terminal cysteine corresponding to Cys-1 of a mature hedgehog. For example, it has been reported that deletions of about 10 contiguous amino acids will provide suitable functional antagonists and that one can also remove fewer than 10 contiguous residues and still maintain antagonist function. It has been further reported that one can delete various combinations of noncontiguous residues provided that there are preferably at least about 3 deleted residues in total.

N-Terminal Mutations

Yet another example of a functional antagonist has a mutation of the N-terminal cysteine to another amino acid residue. Any non-hydrophobic amino acid residue may be acceptable and persons having ordinary skill in the art following the teachings described herein will be able to perform the mutations and test the effects of such mutations. One example is Shh in which the N-terminal cysteine is replaced with a serine residue. Replacements with aspartic acid, alanine and histidine have also reportedly been shown to serve as antagonists.

N-Terminal Cysteine Modifications

Because the primary amino acid sequence of hedgehog contains the Cys-1 that is important for biological activity, many other types of modifications will result in inactive antagonist variants of hedgehog protein. For example, another reported antagonist is an isolated functional antagonist of a hedgehog polypeptide, comprising a hedgehog polypeptide containing an N-terminal cysteine that corresponds to Cys-1 of a mature Sonic hedgehog, except that the cysteine is in a modified form. Antagonist polypeptides of hedgehog may have nonsequence modifications that include in vivo or in vitro chemical derivatization of their N-terminal cysteine, as well as possible changes in acetylation, methylation, phosphorylation, amidation, or carboxylation. As an example, the functional antagonist can have an N-terminal cysteine in an oxidized form. Thus, a functional antagonist can have an N-terminal cysteine that is effectively modified by including it as part of an N-terminal extension moiety.

Antibody Homologs as Modulators

In further embodiments, the modulators used in the method of the invention may be antibodies which bind to, including block or coat, cell-surface hedgehog (such as vertebrate Sonic, Indian or Desert) and/or cell surface ligand for said hedgehog proteins (such as patched) is an anti-hedgehog and/or anti patched monoclonal antibody or antibody homolog. Preferred antibodies and homologs for treatment, in particular for human treatment, include for example human antibody homologs, humanized antibody homologs, chimeric antibody homologs, Fab, Fab′, F(ab′) 2 and F (v) antibody fragments, and monomers or dimers of antibody heavy or light chains or mixtures thereof.

The technology for producing monoclonal antibodies is well known. The preferred antibody homologs contemplated herein can be expressed from intact or truncated genomic or cDNA or from synthetic DNAs in prokaryotic or eukaryotic hosT-cells. The dimeric proteins can be isolated from the culture media and/or refolded and dimerized in vitro to form biologically active compositions. Heterodimers can be formed in vitro by combining separate, distinct polypeptide chains. Alternatively, heterodimers can be formed in a single cell by co-expressing nucleic acids encoding separate, distinct polypeptide chains (see, for example, W093/09229, or U.S. Pat. No. 5,411,941, for several exemplary recombinant heterodimer protein production protocols).

Anti-hedgehog antibodies may, for example, be identified by flow cytometry, e. g., by measuring fluorescent staining of cells incubated with an antibody believed to recognize hedgehog protein. The lymphocytes used in the production of hybridoma cells typically may be isolated from immunized mammals whose sera have already tested positive for the presence of anti-hedgehog antibodies using such screening assays.

Typically, the immortal cell line (e. g., a myeloma cell line) is derived from the same mammalian species as the lymphocytes. Suitable immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion may then be selected using HAT medium, which kills unfused and unproductively fused myeloma cells (unfused splenocytes die after several days because they are not transformed).

Hybridomas producing a desired antibody may be detected by screening the hybridoma culture supernatants. For example, hybridomas prepared to produce anti-hedgehog or patched antibodies may be screened by testing the hybridoma culture supernatant for secreted antibodies having the ability to bind to a recombinant hedgehog or patched expressing cell line.

To produce antibody homologs that are intact immunoglobulins, hybridoma cells that tested positive in such screening assays may be cultured in a nutrient medium under conditions and for a time sufficient to allow the hybridoma cells to secrete the monoclonal antibodies into the culture medium. Tissue culture techniques and culture media suitable for hybridoma cells are well known. The conditioned hybridoma culture supernatant may be collected and the anti-hedgehog or patched antibodies optionally further purified by well-known methods.

Alternatively, the desired antibody may be produced by injecting the hybridoma cells into the peritoneal cavity of an unimmunized mouse. The hybridoma cells may proliferate in the peritoneal cavity, secreting the antibody which accumulates as ascites fluid. The antibody may be harvested by withdrawing the ascites fluid from the peritoneal cavity with a syringe. Several anti-hedgehog or patched monoclonal antibodies have been previously described. These anti-hedgehog or patched monoclonal antibodies and others will be useful in the practice of the present invention.

Fully human monoclonal antibody homologs against hedgehog or patched provide another example of a suitable binding agent which may block or coat hedgehog ligands in the practice of the invention. In their intact form these may, for example, be prepared using in vitro primed human splenocytes, as described by Boerner et al., 1991, J. Immunol., 14, 8695. Alternatively, they may be prepared by repertoire cloning as described by Persson et al., 1991, Proc. Nat. Acad. Sci. USA, 88: 2432-2436 or by Huang and Stollar, 1991, J. Immunol. Methods 141, 227-236; or U.S. Pat. No. 5,798,230.

In one embodiment an antibody suitable for use in the present invention may be a humanized recombinant antibody homolog having, for example, anti-hedgehog or anti-patched specificity. Such antibodies may be produced, for example, as described in EP 0239400 (Winter et al.) whereby antibodies are altered by substitution (within a given variable region) of their complementarity determining regions (CDRs) for one species with those from another. This process may be used, for example, to substitute the CDRs from human heavy and light chain Ig variable region domains with alternative CDRs from murine variable region domains. These altered Ig variable regions may subsequently be combined with human Ig constant regions to create antibodies which are totally human in composition except for the substituted murine CDRs. The process for humanizing monoclonal antibodies via CDR “grafting” has been termed “reshaping”. (Riechmann et al., 1988, Nature 332,323-327; Verhoeyen et al., 1988, Science 239,1534-1536).

Typically, complementarity determining regions (CDRs) of a murine antibody may be transplanted onto the corresponding regions in a human antibody, since it is the CDRs (three in antibody heavy chains, three in light chains) that are the regions of the mouse antibody which bind to a specific antigen. Transplantation of CDRs is achieved by genetic engineering whereby CDR DNA sequences may be determined by cloning of murine heavy and light chain variable (V) region gene segments, and may then be transferred to corresponding human V regions by site directed mutagenesis. Human constant region gene segments of the desired isotype (usually gamma I for CH and kappa for CL) may be added and the humanized heavy and light chain genes may be co-expressed in mammalian cells to produce soluble humanized antibody.

The transfer of CDRs to a human antibody may confer on the human antibody the antigen binding properties of the original murine antibody. The six CDRs in the murine antibody are mounted structurally on a V region “framework” region. Thus, for example, humanized antibody homologs may be prepared, as exemplified in Jones et al., 1986, Nature 321,522-525; Riechmann, 1988, Nature 332,323-327; Queen et al., 1989, Proc. Nat. Acad. Sci. USA 86,10029; and Orlandi et al., 1989, Proc. Nat. Acad. Sci. USA 86,3833. Queen et al., 1989 (supra) and WO 90/07861 (Protein Design Labs) describe the preparation of a humanized antibody that contains modified residues in the framework regions of the acceptor antibody by combining the CDRs of a murine MAb (anti-Tac) with human immunoglobulin framework and constant regions (see also U.S. Pat. Nos. 5,693,762; 5,693,761; 5,585,089; and 5,530,101 (Protein Design Labs)).

Specific examples of antibodies which bind specifically to Patched proteins are described, for example, in U.S. Pat. No. 6,172,200 (The Board of Trustees of the Leland Stanford University) and examples of antibodies which bind specifically to Smoothened proteins are described, for example, in U.S. Pat. No. 6,136,958 (Genentech).

Small Molecule Modulators

Alternatively or in addition, the modulator of the hedgehog signalling pathway may be a so-called “small molecule” agent, typically an organic molecule having a molecular weight of less than 2000 Da, preferably less than 1000 Da, suitably less than 500 Da. Many examples of such compounds are known in the art, for example as follows:

In one embodiment, the modulator of the hedgehog signalling pathway may, for example, be a compound as described in WO 01/74344 (Curis), for example as represented by the general formula VIII:

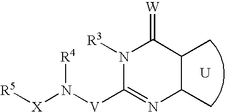

wherein:

U represents a substituted or unsubstituted aryl or heteroaryl ring fused to the nitrogen-containing ring;

V represents a lower alkylene group;

W represents S or O, preferably O;

X represents C═O, C═S, or SOx;

R³ represents substituted or unsubstituted aryl, heteroaryl, lower alkyl, lower alkenyl, lower alkynyl, carbocyclyl, carbocyclylalkyl, heterocyclyl, heterocyclylalkyl, aralkyl, or heteroaralkyl;

R⁴ represents substituted or unsubstituted aralkyl or lower alkyl; and

R⁵ represents substituted or unsubstituted aryl, heteroaryl, aralkyl, or heteroaralkyl, including polycyclic aromatic or heteroaromatic groups.

Alternatively or in addition the modulator of the hedgehog signalling pathway may, for example, be a compound as described in WO 01/74344 (Curis), for example as represented by the general formula (I):

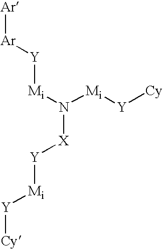

wherein:

Ar and Ar′ independently represent substituted or unsubstituted aryl or heteroaryl rings;

Y, independently for each occurrence, is absent or represents —N(R)—, —O—, —S— or —Se—;

X is selected from —C(═O)—, —C(═S)—, —S(0₂)-, —S(O)—, —C(═NCN)—, —P(═O)(OR)—, and a methylene group optionally substituted with 1-2 groups selected from lower alkyl, alkenyl, and alkynyl groups;

M represents, independently for each occurrence, a substituted or unsubstituted methylene group, or two M taken together represent substituted or unsubstituted ethene or ethyne;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4-to 8-membered ring;

Cy and Cy′ independently represent substituted or unsubstituted aryl, heterocyclyl, heteroaryl, or cycloalkyl;

i represents, independently for each occurrence, an integer from 0 to 5; and

n, individually for each occurrence, represents an integer from 0 to 10.

Alternatively or in addition the modulator of the hedgehog signalling pathway may, for example, be a compound as described in WO 01/74344 (Curis), for example as represented by the general formula (II):

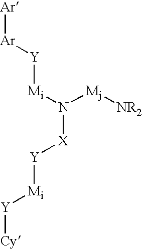

wherein:

Ar and Ar′ independently represent substituted or unsubstituted aryl or heteroaryl rings;

Y, independently for each occurrence, is absent or represents —N(R)—, —O—, —S—, or —Se—;

X is selected from —C(═O)—, —C(═S)—, —S(0₂)-, —S(O)—, —C(═NCN)—, —P(═O)(OR)—, and a methylene group optionally substituted with 1-2 groups selected from lower alkyl, alkenyl, and alkynyl groups;

M represents, independently for each occurrence, a substituted or unsubstituted methylene group, or two M taken together represent substituted or unsubstituted ethene or ethyne, wherein some or all occurrences of M in Mj form all or part of a cyclic structure;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4-to 8-membered ring;

Cy′ represents substituted or unsubstituted aryl, heterocyclyl, heteroaryl, or cycloalkyl;

j represents, independently for each occurrence, an integer from 0 to 10;

i represents, independently for each occurrence, an integer from 0 to 5; and

n, individually for each occurrence, represents an integer from 0 to 10.

Alternatively or in addition the modulator of the hedgehog signalling pathway may, for example, be a compound as described in WO 01/74344 (Curis), for example as represented by the general formula (III):

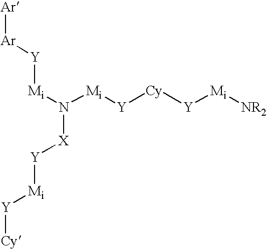

wherein:

Ar and Ar′ independently represent substituted or unsubstituted aryl or heteroaryl rings;

Y, independently for each occurrence, is absent or represents —N(R)—, —O—, —S—, or —Se—;

X is selected from —C(═O)—, —C(═S)—, —S(0₂)-, —S(O)—, —C(═NCN)—, —P(═O)(OR)—, and a methylene group optionally substituted with 1-2 groups selected from lower alkyl, alkenyl, and alkynyl groups;

M represents, independently for each occurrence, a substituted or unsubstituted methylene group, or two M taken together represent substituted or unsubstituted ethene or ethyne, wherein some or all occurrences of M in Mj form all or part of a cyclic structure;

R represents, independently for each occurrence, H or substituted or unsubstituted aryl, heterocyclyl, heteroaryl, aralkyl, heteroaralkyl, alkynyl, alkenyl, or alkyl, or two R taken together may form a 4-to 8-membered ring;

Cy and Cy′ independently represent substituted or unsubstituted aryl, heterocyclyl, heteroaryl, or cycloalkyl;

i represents, independently for each occurrence, an integer from 0 to 5; and n, individually for each occurrence, represents an integer from 0 to 10.

Alternatively or in addition, the modulator of the hedgehog signalling pathway may, for example, be a compound as described in WO 01/74344 (Curis), for example as represented by the general formula (IX) of WO01/74344:

wherein:

Ar represents a substituted or unsubstituted aryl or heteroaryl ring;

Z is absent or represents a substituted or unsubstituted aryl, carbocyclyl, heterocyclyl, or heteroaryl ring, or a nitro, cyano, or halogen substituent;

Y, independently for each occurrence, is absent or represents —N(R)—, —O—, —S—, or —Se—, provided that if Z is not a ring, then Y attached to Z is absent;

X is selected from —C(═O)—, —C(═S)—, —S(0₂)-, —S(O)—, —C(═NCN)—, —P(═O)(OR)— and a methylene group optionally substituted with 1-2 groups;

M represents, independently for each occurrence, a substituted or unsubstituted methylene group, or two M taken together represent substituted or unsubstituted ethene or ethyne;

R represents, independently for each occurrence, H or substituted or unsubstituted aryl, heterocyclyl, carbocyclyl, heteroaryl, aralkyl, heteroaralkyl, heterocyclylalkyl, carbocyclylalkyl, alkynyl, alkenyl, or alkyl, or two R taken together may form a 4-to 8-membered ring;

Cy and Cy′ independently represent substituted or unsubstituted aryl, heterocyclyl, heteroaryl, or cycloalkyl, including polycyclic groups;

i represents, independently for each occurrence, an integer from 0 to 5; and

k represents an integer from 0 to 3.

Alternatively or in addition the modulator of the hedgehog signalling pathway may, for example, be a compound as described in WO 01/74344 (Curis), for example as represented by the general formula (X) of this publication:

wherein:

Ar represents a substituted or unsubstituted aryl or heteroaryl ring;

Z is absent or represents a substituted or unsubstituted aryl, carbocyclyl, heterocyclyl, or heteroaryl ring, or a nitro, cyano, or halogen substituent;

Y, independently for each occurrence, is absent or represents —N(R)—, —O—, —S— or —Se—, provided that if Z is not a ring, then Y attached to Z is absent;

X is selected from —C(═O)—, —C (═S)—, —S(0₂)-, —S(O)—, —C(═NCN)—, —P (═O)(OR)— and a methylene group optionally substituted with 1-2 groups;

R represents, independently for each occurrence, H or substituted or unsubstituted aryl, heterocyclyl, carbocyclyl, heteroaryl, aralkyl, heteroaralkyl, heterocyclylalkyl, carbocyclylalkyl, alkynyl, alkenyl, or alkyl, or two R taken together may form a 4-to 8-membered ring;

Cy′ represents a substituted or unsubstituted aryl, heterocyclyl, heteroaryl, or cycloalkyl, including polycyclic groups;

M represents, independently for each occurrence, a substituted or unsubstituted methylene group, or two M taken together represent substituted or unsubstituted ethene or ethyne, wherein some or all occurrences of M in Mj form all or part of a cyclic structure;

j represents, independently for each occurrence, an integer from 2 to 10;

i represents, independently for each occurrence, an integer from 0 to 5; and

k represents, independently for each occurrence, an integer from 0 to 3.

Alternatively or in addition, the modulator of the hedgehog signalling pathway may, for example, be a compound as described in WO 01/74344 (Curis), for example as represented by the general formula (XI) of this publication:

wherein:

Ar represents a substituted or unsubstituted aryl or heteroaryl ring;

Z is absent or represents a substituted or unsubstituted aryl, carbocyclyl, heterocyclyl, or heteroaryl ring, or a nitro, cyano, or halogen substituent;

Y, independently for each occurrence, is absent or represents —N(R)—, —O—, —S— or —Se—, provided that if Z is not a ring, then Y attached to Z is absent;

X is selected from —C(═O)—, —C(═S)—, —S(0₂)-, —S(O)—, —C(═NCN)—, —P(═O)(OR)— and a methylene group optionally substituted with 1-2 groups;

M represents, independently for each occurrence, a substituted or unsubstituted methylene group, or two M taken together represent substituted or unsubstituted ethene or ethyne;

R represents, independently for each occurrence, H or substituted or unsubstituted aryl, heterocyclyl, carbocyclyl, heteroaryl, aralkyl, heteroaralkyl, heterocyclylalkyl, carbocyclylalkyl, alkynyl, alkenyl, or alkyl, or two R taken together may form a 4-to 8-membered ring;

Cy and Cy′ independently represent substituted or unsubstituted aryl, heterocyclyl, heteroaryl, or cycloalkyl, including polycyclic groups;

i represents, independently for each occurrence, an integer from 0 to 5; and k represents an integer from 0 to 3.

Alternatively or in addition, the modulator of the hedgehog signalling pathway may, for example, be a compound as described in WO 01/19800 (Curis), for example as represented by the general formula (I):

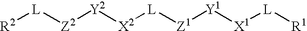

wherein:

R¹ and R², independently for each occurrence, represent H, lower alkyl, —(CH₂)ₙ-aryl (substituted or unsubstituted), or —(CH₂)ₙ-heteroaryl (substituted or unsubstituted);

L, independently for each occurrence, is absent or represents —(CH₂)ₙ-alkyl, -alkenyl-, -alkynyl-, —(CH₂)ₙ-alkenyl-, —(CH₂)ₙ-alkynyl-, —(CH₂)ₙO(CH₂)ₚ—, —(CH₂)ₙNR₂(CH₂)ₚ—, —(CH₂)ₙS(CH₂)ₚ—, —(CH₂)ₙ-alkenyl(CH₂)ₚ—, —(CH₂)ₙ-alkynyl(CH₂)ₚ—, —O(CH₂)ₙ—, —NR₂(CH₂)ₙ—, or —S(CH₂)ₙ—;

X1 and X2 are selected, independently, from —N(R⁸)—, —O—, —S—, —Se—, —N═N—, —ON═CH—, —(R⁸)N—N(^{R8})—, —ON(R⁸)—, a heterocycle, or a direct bond between L and Y¹ or Y², respectively;

Y¹ and Y² are selected, independently, from —C(═O)—, —C(═S)—, —S(02)-, —S(O)—, C(═NCN)—, —P(═O)(OR²)—, a heteroaromatic group, or a direct bond between X¹ and Z¹ or X² and Z², respectively;

Z¹ and Z² are selected, independently, from —N(R⁸)—, —O—, —S—, —Se—, —N═N—, —ON═CH—, —R⁸N—NR⁸—ONR⁸—, a heterocycle, or a direct bond between Y¹ or Y², respectively, and L;

R⁸, independently for each occurrence, represents H, lower alkyl, —(CH₂)ₙ-aryl (substituted or unsubstituted), —(CH₂)ₙ-heteroaryl (substituted or unsubstituted), or two R⁸ taken together form a 4-to 8-membered ring, together with the atoms to which they are attached, which ring may include one or more carbonyls;

p represents, independently for each occurrence, an integer from 0 to 10; and n, individually for each occurrence, represents an integer from 0 to 10.

Alternatively or in addition, the modulator of the hedgehog signalling pathway may, for example, be a compound as described in WO 01/19800 (Curis), for example as represented by the general formula (II):

wherein:

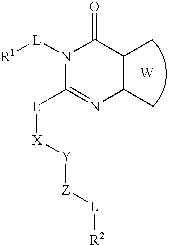

R¹ and R², independently for each occurrence, represent H, lower alkyl, aryl (substituted or unsubstituted), aralkyl (substituted or unsubstituted), heteroaryl (substituted or unsubstituted), or heteroaralkyl (substituted or unsubstituted);

L, independently for each occurrence, is absent or represents —(CH₂)ₙ-alkyl, -alkenyl-, -alkynyl-, —(CH₂)ₙalkenyl-, —(CH₂)ₙalkynyl-, —(CH₂)ₙO(CH₂)ₚ—, —(CH₂)ₙNR₂(CH₂)ₚ—, —(CH₂)ₙS(CH₂)ₚ—, —(CH₂)ₙalkenyl(CH₂)ₚ—, —(CH₂)ₙalkynyl (CH₂)ₚ—, —O(CH₂)ₙ—, —NR₂(CH₂)ₙ—, or —S(CH₂)ₙ—;

X is selected from —N(R⁸)—, —O—, —S—, —Se—, —N═N—, —ON═CH—, —(R⁸)—N—N—(R⁸)—, —ON—(R⁸)—, a heterocycle, or a direct bond between L and Y;

Y is selected from —C(═O)—, —C(═S)—, —S(0₂)-, —S(O)—, —C(═NCN)—, —P(═O)(OR₂)—, a heteroaromatic group, or a direct bond between X and Z;

Z is selected from —N(R⁸)—, —O—, —S—, —Se—, —N═N—, —ON═CH—, —R⁸—N—N—R⁸—, —ONR⁸—, a heterocycle, or a direct bond between Y and L;

R⁸, independently for each occurrence, represents H, lower alkyl, aryl (substituted or unsubstituted), aralkyl (substituted or unsubstituted), heteroaryl (substituted or unsubstituted), or heteroaralkyl (substituted or unsubstituted), or two R⁸ taken together form a 4-to 8-membered ring, together with the atoms to which they are attached, which ring may include one or more carbonyls;

W represents a substituted or unsubsituted aryl or heteroaryl ring fused to the pyrimidone ring; p represents, independently for each occurrence, an integer from 0 to 10; and n, individually for each occurrence, represents an integer from 0 to 10.

The term “aliphatic group” as used herein refers to a straight-chain, branched-chain, or cyclic aliphatic hydrocarbon group and includes saturated and unsaturated aliphatic groups, such as an alkyl group, an alkenyl group, and an alkynyl group.

The terms “alkenyl” and “alkynyl” refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R⁸, where m and R⁸ are as described herein.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e. g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters),—CF₃,—CN and the like. Exemplary substituted alkyls are described below. Cycloalkyls can be further substituted with alkyls, alkenyls, alkoxys, alkylthios, aminoalkyls, carbonyl-substituted alkyls,—CF₃,—CN, and the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and-S—(CH₂)ₘ—R⁸, wherein m and R⁸ are as defined herein. Representative alkylthio groups include methylthio, ethylthio, and the like.

The term “aralkyl”, as used herein, refers to an alkyl group substituted with an aryl group (e. g., an aromatic or heteroaromatic group).

The term “aryl” as used herein includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” groups, “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃,—CN, or the like. The term “aryl” also includes polycyclic ring systems having two or more cyclic rings in which two or more carbons are common to two adjoining rings (the rings are “fused rings”) wherein at least one of the rings is aromatic, e. g., the other cyclic rings can be cycloalkyls, cycloalkenyls, cycloalkynyls, aryls and/or heterocyclyls.

The term “carbocycle”, as used herein, refers to an aromatic or non-aromatic ring in which each atom of the ring is carbon.

The term “heteroatom” as used herein means an atom of any element other than carbon or hydrogen. Preferred heteroatoms are boron, nitrogen, oxygen, phosphorus, sulfur and selenium.

The terms “heterocyclyl” or “heterocyclic group” refer to 3-to 10-membered ring structures, more preferably 3-to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety,—CF₃,—CN, or the like.

As used herein, the term “halogen“designates —F, Cl, —Br or —I;

The terms “polycyclyl” or “polycyclic group” refer to two or more rings (e. g., cycloalkyls, cycloalkenyls, cycloalkynyls, aryls and/or heterocyclyls) in which two or more carbons are common to two adjoining rings, e. g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety,—CF₃,—CN, or the like.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e. g., which does not spontaneously undergo transformation such as by rearrangement, cyclization, elimination, etc.

Lead Compound Discovery/High-Throughput Screening Assay

An example of a high throughput screen suitable for testing or confirming the activity of modulators of hedgehog signalling is described in WO 0119800 (Curis) as follows:

Compounds to be tested are dissolved in DMSO to a concentration of 10 mM, and stored at −20 C. To activate the Hedgehog pathway in the assay cells, an octylated (lipid-modified) form of the N-terminal fragment of the Sonic Hedgehog protein (OCT-SHH) is used. This N-terminal SHH fragment is produced bacterially.

Compounds may be tested in the“Gli-Luc”assay below, using the cell line 10T (s12), wherein the cells contain a Hedgehog-responsive reporter construct utilizing Luciferase as the reporter gene. In this way, any increase or decrease in Hedgehog pathway signaling activity can be measured via the Gli-Luc response.

10tl/2 (s12) cells are plated in a 96-well micro-titer plate (MTP) at 20,000 cells/well in full medium [DMEM with 10% FBS]. Then plates are placed in the incubator for incubation overnight (O/N), at 37 C and 5% CO2. After 24 h, the medium is replaced with Luciferase-assay medium (DMEM with 0.5% FBS). Compounds are thawed and diluted in assay medium at 3: 1000 (about 300-fold) resulting in a starting concentration of about 30 IlM. Subsequently, 150 l of each 30 iM sample is added to the first wells (in triplicate).

The MTP samples are then diluted at 3-fold dilutions to a total of seven wells, ultimately resulting in a regimen of seven dilutions in triplicate, for each compound. Next, the protein ligand OCT-SHH is diluted in Luciferase-assay medium and added to each well at a final concentration of 0.3 pg/ml. Plates are then returned to the incubator for further incubation O/N, at 37 C and 5% CO2. After about 24 h, plates are removed from the incubator and the medium is aspirated/discarded. Wells are washed once with assay buffer [PBS +1 mM Mg2+ and 1 mM Ca2+]. Then 50 l of assay buffer is added to each well. The Luciferase assay reagent is prepared as described by the vendor (LucLite kit from Packard), and 50 ul is added to each well. Plates are incubated at room temperature (RT) for about 30 minutes after which the signals are read, again at RT, on a Topcount (Packard) to determine hedgehog signalling.

By comparing the level of signalling both with and without a given compound, the action of the compound in increasing or decreasing signalling activity can be readily evaluated.

Transgenic Animals

The present invention also relates to transgenic animals which are capable of expressing or overexpressing at least one modulator useful in the present invention. Preferably the animal expresses or overexpresses HIP, Frzb-1 and/or WIF-1.

The present invention additionally relates to transgenic animals which are capable of expressing or overexpressing at least one polypeptide which is a component of the Hedgehog signalling pathway or a component of a pathway which is a target of the Hedgehog signalling pathway, such as the Wnt signalling pathway. Preferably the animal expresses or overexpresses HH (more preferably Shh), and/or Dvl-1.

The transgenic animal is typically a vertebrate, more preferably a rodent, such as a rat or a mouse, but also includes other mammals such as human, goat, pig or cow etc.

Such transgenic animals are useful as animal models of disease and in screening assays for new useful compounds. By specifically expressing one or more polypeptides, as defined above, the effect of such polypeptides on the development of disease can be studied. Furthermore, therapies including gene therapy and various drugs can be tested on transgenic animals. Methods for the production of transgenic animals are known in the art. For example, there are several possible routes for the introduction of genes into embryos. These include (i) direct transfection or retroviral infection of embryonic stem cells followed by introduction of these cells into an embryo at the blastocyst stage of development; (ii) retroviral infection of early embryos; and (iii) direct microinjection of DNA into zygotes or early embryo cells.

The present invention also includes stable cell lines for use as disease models for testing or treatment.

A stable cell line will contain a recombinant gene or genes, also known herein as a transgene, encoding one or more inhibitors or components of a Hedgehog signalling pathway or of a pathway which is a target of the Hedgehog signalling pathway.

Preferably the transgene is HH (more preferably Shh), HIP, WIF-1, Frzb-1, Ngg and/or Dvl-1. A cell line containing a transgene, as described herein, is made by introducing the transgene into a selected cell line according to one of several procedures known in the art for introducing a foreign gene into a cell.

As also described below, the sequences encoding the modulators and components of signalling pathways, as described herein, are operably linked to control sequences, including promoters/enhancers and other expression regulation signals.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymptocytes, dendritic cells, skin, brain cells and epithelial cells within the eye are particularly preferred, for example the CD2, CD11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Assays

Assays for monitoring expression of the one or more target genes and other methods of detecting modulation of Hedgehog signalling are described below.

The present invention preferably provides a cell-based assay for screening compounds for their ability to modulate Hedgehog signalling. In one embodiment, the present invention provides an assay comprising the steps of:

(a) providing a culture of immune cells;

(b) optionally transfecting said cells with a reporter construct;

(c) optionally transfecting said cells with a Hedgehog gene;

(d) exposing the cells to one or more compound(s) to be tested; and

(e) determining the difference in Hedgehog signalling between cells exposed to the compound(s) to be tested and cells not so exposed.

The assay of the present invention is set up to detect either inhibition or enhancement of Hedgehog signalling in cells of the immune system by candidate modulators. The method comprises mixing cells of the immune system, where necessary transformed or transfected, etc. with a synthetic reporter gene, in an appropriate buffer, with a sufficient amount of candidate modulator and monitoring Hedgehog signalling. The modulators may be small molecules, proteins, antibodies or other ligands as described above. Amounts or activity of the target gene (also described above) will be measured for each compound tested using standard assay techniques and appropriate controls. Preferably the detected signal is compared with a reference signal and any modulation with respect to the reference signal measured.

The assay may also be run in the presence of a known antagonist of the Hedgehog signalling pathway in order to identify compounds capable of rescuing the Hedgehog signal.

Any one or more of appropriate targets—such as an amino acid sequence and/or nucleotide sequence—may be used for identifying a compound capable of modulating the Hedgehog signalling pathway in cells of the immune system in any of a variety of drug screening techniques. The target employed in such a test may be free in solution, affixed to a solid support, borne on a cell surface, or located intracellularly. The assay of the present invention is a cell based assay.

The assay of the present invention may be a screen, whereby a number of agents are tested. In one aspect, the assay method of the present invention is a high through put screen.

Techniques for drug screening may be based on the method described in Geysen, European Patent No. 0138855, published on Sep. 13, 1984. In summary, large numbers of different small peptide candidate modulators are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators.

This invention also contemplates the use of competitive drug screening assays in which neutralising antibodies capable of binding a target specifically compete with a test compound for binding to a target.

It is expected that the assay methods of the present invention will be suitable for both small and large-scale screening of test compounds as well as in quantitative assays.

Various nucleic acid assays are also known. Any conventional technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

Target gene presence, amplification and/or expression may be measured in a sample directly, for example, by conventional Southern blotting, Northern blotting to quantitate the transcription of target mRNA, dot blotting (DNA or RNA analysis), or in situ hybridisation, using an appropriately labelled probe. Those skilled in the art will readily envisage how these methods may be modified, if desired.

Generation of nucleic acids for analysis from samples generally requires nucleic acid amplification. Many amplification methods rely on an enzymatic chain reaction (such as a polymerase chain reaction, a ligase chain reaction, or a self-sustained sequence replication) or from the replication of all or part of the vector into which it has been cloned. Preferably, the amplification according to the invention is an exponential amplification, as exhibited by for example the polymerase chain reaction.

Many target and signal amplification methods have been described in the literature, for example, general reviews of these methods in Landegren, U., et al., Science 242:229-237 (1988) and Lewis, R., Genetic Engineering News 10:1, 54-55 (1990). These amplification methods may be used in the methods of our invention, and include polymerase chain reaction (PCR), PCR in situ, ligase amplification reaction (LAR), ligase hybridisation, Qbeta bacteriophage replicase, transcription-based amplification system (TAS), genomic amplification with transcript sequencing (GAWTS), nucleic acid sequence-based amplification (NASBA) and in situ hybridisation. Primers suitable for use in various amplification techniques can be prepared according to methods known in the art.

PCR is a nucleic acid amplification method described inter alia in U.S. Pat. Nos. 4,683,195 and 4,683,202. PCR consists of repeated cycles of DNA polymerase generated primer extension reactions. PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached. PCR can be used to amplify any known nucleic acid in a diagnostic context (Mok et al., (1994), Gynaecologic Oncology, 52: 247-252).

Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 10⁶ to 10⁹ have been achieved in one hour at 42° C.

Ligation amplification reaction or ligation amplification system uses DNA ligase and four oligonucleotides, two per target strand. This technique is described by Wu, D. Y. and Wallace, R. B. (1989) Genomics 4:560. The oligonucleotides hybridise to adjacent sequences on the target DNA and are joined by the ligase. The reaction is heat denatured and the cycle repeated.

Alternative amplification technology can be exploited in the present invention. For example, rolling circle amplification (Lizardi et al., (1998) Nat Genet 19:225) is an amplification technology available commercially (RCAT™) which is driven by DNA polymerase and can replicate circular oligonucleotide probes with either linear or geometric kinetics under isothermal conditions.

In the presence of two suitably designed primers, a geometric amplification occurs via DNA strand displacement and hyperbranching to generate 10¹² or more copies of each circle in 1 hour.

If a single primer is used, RCAT generates in a few minutes a linear chain of thousands of tandemly linked DNA copies of a target covalently linked to that target.

A further technique, strand displacement amplification (SDA; Walker et al., (1992) PNAS (USA) 80:392) begins with a specifically defined sequence unique to a specific target. But unlike other techniques which rely on thermal cycling, SDA is an isothermal process that utilises a series of primers, DNA polymerase and a restriction enzyme to exponentially amplify the unique nucleic acid sequence.

SDA comprises both a target generation phase and an exponential amplification phase.

In target generation, double-stranded DNA is heat denatured creating two single-stranded copies. A series of specially manufactured primers combine with DNA polymerase (amplification primers for copying the base sequence and bumper primers for displacing the newly created strands) to form altered targets capable of exponential amplification.

The exponential amplification process begins with altered targets (single-stranded partial DNA strands with restricted enzyme recognition sites) from the target generation phase.

An amplification primer is bound to each strand at its complementary DNA sequence. DNA polymerase then uses the primer to identify a location to extend the primer from its 3′ end, using the altered target as a template for adding individual nucleotides. The extended primer thus forms a double-stranded DNA segment containing a complete restriction enzyme recognition site at each end.

A restriction enzyme is then bound to the double stranded DNA segment at its recognition site. The restriction enzyme dissociates from the recognition site after having cleaved only one strand of the double-sided segment, forming a nick. DNA polymerase recognises the nick and extends the strand from the site, displacing the previously created strand. The recognition site is thus repeatedly nicked and restored by the restriction enzyme and DNA polymerase with continuous displacement of DNA strands containing the target segment.

Each displaced strand is then available to anneal with amplification primers as above. The process continues with repeated nicking, extension and displacement of new DNA strands, resulting in exponential amplification of the original DNA target.

In an alternative embodiment, the present invention provides for the detection of gene expression at the RNA level. Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

PCR technology as described e.g. in section 14 of Sambrook et al., 1989, requires the use of oligonucleotide probes that will hybridise to target nucleic acid sequences. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating ³²P dATP with the Klenow fragment of DNA polymerase in a random priming reaction, as is well known in the art. Oligonucleotides are usually end-labelled with ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation. Preferred are such sequences, probes which hybridise under high-stringency conditions.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefor easily identifiable. Thus, cell-based screening assays can be designed by constructing cell lines in which the expression of a reporter protein, i.e. an easily assayable protein, such as β-galactosidase, chloramphenicol acetyltransferase (CAT) or luciferase, is dependent on the activation of a Hedgehog. For example, a reporter gene encoding one of the above polypeptides may be placed under the control of an response element which is specifically activated by Hedgehog signalling. Alternative assay formats include assays which directly assess responses in a biological system. If a cell-based assay system is employed, the test compound(s) indentified may then be subjected to in vivo testing to determine their effect on Hedgehog signalling pathway.

In general, reporter constructs useful for detecting Hedgehog signalling by expression of a reporter gene may be constructed according to the general teaching of Sambrook et al (1989). Typically, constructs according to the invention comprise a promoter of the gene of interest (i.e. of an endogenous target gene), and a coding sequence encoding the desired reporter constructs, for example of GFP or luciferase. Vectors encoding GFP and luciferase are known in the art and available commercially. Reporter genes are discussed in more detail below.

Sorting of cells, based upon detection of expression of target genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure target gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefor generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefor assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a target mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In Vivo Expression Technology (IVET) (Camilli). IVET identifies target genes up-regulated during say treatment or disease when compared to laboratory culture.

The present invention also provides a method of detection of polypeptides. The advantage of using a protein assay is that Hedgehog activation can be directly measured. Assay techniques that can be used to determine levels of a polypeptide are well known to those skilled in the art. Such assay methods include radioimmunoassays, competitive-binding assays, protein gel assay, Western Blot analysis, antibody sandwich assays, antibody detection, FACS and ELISA assays. For example, polypeptides can be detected by differential mobility on protein gels, or by other size analysis techniques, such as mass spectrometry. The detection means may be sequence-specific. For example, polypeptide or RNA molecules can be developed which specifically recognise polypeptides in vivo or in vitro.

For example, RNA aptamers can be produced by SELEX. SELEX is a method for the in vitro evolution of nucleic acid molecules with highly specific binding to target molecules. It is described, for example, in U.S. Pat. Nos. 5,654,151, 5,503,978, 5,567,588 and 5,270,163, as well as PCT publication WO 96/38579

The invention, in certain embodiments, includes antibodies specifically recognising and binding to polypeptides.

Antibodies may be recovered from the serum of immunised animals. Monoclonal antibodies may be prepared from cells from immunised animals in the conventional manner.

The antibodies of the invention are useful for identifying cells expressing the genes being monitored.

Antibodies according to the invention may be whole antibodies of natural classes, such as IgE and IgM antibodies, but are preferably IgG antibodies. Moreover, the invention includes antibody fragments, such as Fab, F(ab′)2, Fv and ScFv. Small fragments, such Fv and ScFv, possess advantageous properties for diagnostic and therapeutic applications on account of their small size and consequent superior tissue distribution.

The antibodies may comprise a label. Especially preferred are labels which allow the imaging of the antibody in neural cells in vivo. Such labels may be radioactive labels or radioopaque labels, such as metal particles, which are readily visualisable within tissues. Moreover, they may be fluorescent labels or other labels which are visualisable in tissues and which may be used for cell sorting.

In more detail, antibodies as used herein can be altered antibodies comprising an effector protein such as a label. Especially preferred are labels which allow the imaging of the distribution of the antibody in vivo. Such labels can be radioactive labels or radioopaque labels, such as metal particles, which are readily visualisable within the body of a patient. Moreover, they can be fluorescent labels or other labels which are visualisable on tissue Antibodies as described herein can be produced in cell culture. Recombinant DNA technology can be used to produce the antibodies according to established procedure, in bacterial or preferably mammalian cell culture. The selected cell culture system optionally secretes the antibody product, although antibody products can be isolated from non-secreting cells.

Multiplication of hybridoma cells or mammalian hosT-cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of hosT-cells which are bacterial cells or yeasT-cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by an enzyme immunoassay, e.g. a sandwich assay or a dot-assay, or a radioinmuunoassay.

For isolation of the antibodies, the immunoglobulins in the culture supernatants or in the ascitic fluid can be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with the target antigen, or with Protein-A.

The antibody is preferably provided together with means for detecting the antibody, which can be enzymatic, fluorescent, radioisotopic or other means. The antibody and the detection means can be provided for simultaneous, simultaneous separate or sequential use, in a kit.

The antibodies of the invention are assayed for immunospecific binding by any method known in the art. The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al, eds, 1994, Current Protocols in Molecular Biology, Vol. 1, John Wiley & Sons, Inc., New York, which is incorporated by reference herein in its entirety). Exemplary immunoassays are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2,1% Trasylol) supplemented with protein phosphatase and/or protease inhibitors (e. g., EDTA, PMSF, aprotinin, sodium vanadate), adding the antibody of interest to the cell lysate, incubating for a period of time (e. g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e. g., western blot analysis.

Western blot analysis generally comprises preparing protein samples, electrophoresis of the protein samples in a polyacrylamide gel (e. g., 8%-20% SDS-PAGE depending on the molecular weight of the antigen), transferring the protein sample from the polyacrylamide gel to a membrane such as nitrocellulose, PVDF or nylon, blocking the membrane in blocking solution (e. g., PBS with 3% BSA or non-fat milk), washing the membrane in washing buffer (e. g., PBS-Tween 20), exposing the membrane to a primary antibody (the antibody of interest) diluted in blocking buffer, washing the membrane in washing buffer, exposing the membrane to a secondary antibody (which recognises the primary antibody, e. g., an antihuman antibody) conjugated to an enzymatic substrate (e. g., horseradish peroxidase or alkaline phosphatase) or radioactive molecule (e. g., ³²P or ¹²⁵I) diluted in blocking buffer, washing the membrane in wash buffer, and detecting the presence of the antigen.

ELISAs generally comprise preparing antigen, coating the well of a 96 well microtitre plate with the antigen, adding the antibody of interest conjugated to a detectable compound such as an enzymatic substrate (e. g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognises the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well.

It is convenient when running assays to immobilise one of more of the reactants, particularly when the reactant is soluble. In the present case it may be convenient to immobilse any one of more of the candidate modulator, Hedgehog ligand, immune cell activator or immune cell costimulus. Immobilisation approaches include covalent immobilsation, such as using amine coupling, surface thiol coupling, ligand thiol coupling and aldehyde coupling, and high affinity capture which relies on high affinity binding of a ligand to an immobilsed capturing molecule. Example of capturing molecules include: streptavidin, anti-mouse Ig antibodies, ligand-specific antibodies, protein A, protein G and Tag-specific capture. In one embodiment, immobilisation is achieved through binding to a support, particularly a particulate support which is preferably in the form of a bead.

For assays involving monitoring or detection of tolerised T-cells for use in clinical applications, the assay will generally involve removal of a sample from a patient prior to the step of detecting a signal resulting from cleavage of the intracellular domain.

The invention additionally provides a method of screening for a candidate modulator of Hedgehog signalling, the method comprising mixing in a buffer an appropriate amount of Hedgehog, wherein Hedgehog is suitably labelled with detection means for monitoring cleavage of Hedgehog; and a sample of a candidate ligand; and monitoring any cleavage of Hedgehog.

As used herein, the term “sample” refers to a collection of inorganic, organic or biochemical molecules which is either found in nature (e.g., in a biological- or other specimen) or in an artificially-constructed grouping, such as agents which may be found and/or mixed in a laboratory. The biological sample may refer to a whole organism, but more usually to a subset of its tissues, cells or component parts (e.g. body fluids, including but not limited to blood, mucus, saliva and urine).

The present invention provides a method of detecting novel modulators of Hedgehog signalling. The modulators identified may be used as therapeutic agents—i.e. in therapy applications.

Cells of the Immune System

Cells of use in the present invention are cells of the immune system capable of transducing the Hedgehog signalling pathway.

Most preferably the cells of use in the present invention are T-cells. These include, but are not limited to, CD4⁺ and CD8⁺ mature T-cells, immature T-cells of peripheral or thymic origin and NK-T-cells.

Alternatively, the cells will be antigen-presenting cells (APCs). APCs include dendritic cells (DCs) such as interdigitating DCs or follicular DCs, Langerhans cells, PBMCs, macrophages, B-lymphocytes, T-lymphocytes, or other cell types such as epithelial cells, fibroblasts or endothelial cells, constitutively expressing or activated to express a MHC Class II molecules on their surfaces. Precursors of APCs include CD34⁺ cells, monocytes, fibroblasts and endothelial cells. The APCs or precursors may be modified by the culture conditions or may be genetically modified, for instance by transfection of one or more genes.

The T-cells or APCs may be isolated from a patient, or from a donor individual or another individual. The cells are preferably mammalian cells such as human or mouse cells. Preferably the cells are of human origin. The APC or precursor APC may be provided by a cell proliferating in culture such as an established cell line or a primary cell culture. Examples include hybridoma cell lines, L-cells and human fibroblasts such as MRC-5. Preferred cell lines for use in the present invention include Jurkat, H9, CEM and EL4 T-cells; long-term T-cell clones such as human HA1.7 or mouse D10 cells; T-cell hybridomas such as DO11.10 cells; macrophage-like cells such as U937 or THP1 cells; B-cell lines such as EBV-transformed cells such as Raji, A20 and M1 cells.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba (1992) J Exp Med 175:1157-1167), or from bone marrow, non-adherent CD34+ cells can be treated with GM-CSF and TNF-α (Caux et al (1992) Nature 360:258-261). DCs can also be routinely prepared from the peripheral blood of human volunteers, similarly to the method of Sallusto and Lanzavecchia J Exp Med (1994) 179(4) 1109-18 using purified peripheral blood mononucleocytes (PBMCs) and treating 2 hour adherenT-cells with GM-CSF and IL-4. If required, these may be depleted of CD19⁺ B cells and CD3⁺ , CD2⁺ T-cells using magnetic beads (Coffin et al (1998) Gene Therapy 5:718-722). Culture conditions may include other cytokines such as GM-CSF or IL-4 for the maintenance and, or activity of the dendritic cells or other antigen presenting cells.

T-cells and B cells for use in the invention are preferably obtained from cell lines such as lymphoma or leukemia cell lines, T-cell hybridomas or B cell hybridomas but may also be isolated from an individual suffering from a disease of the immune system or a recipient for a transplant operation or from a related or unrelated donor individual. T-cells and B cells may be obtained from blood or another source (such as lymph nodes, spleen, or bone marrow) and may be enriched or purified by standard procedures. Alternatively whole blood may be used or leukocyte enriched blood or purified white blood cells as a source of T-cells and other cell types. It is particularly preferred to use helper T-cells (CD4⁺). Alternatively other T-cells such as CD8⁺ cells may be used.

Candidate modulators of use in the present invention are brought into contact with a cell of the immune system as described above. In a further step, modulation of Hedgehog signalling by a candidate modulator is detected. Assays for detecting modulation of Hedgehog signalling will be described below. Many of these assays will involve monitoring the expression of a “target gene”.

Stimulatory Signals

Expression or repression of the target genes (endogenous or reporter genes) of use in the present invention is dependent on Hedgehog signalling. In a preferred embodiment, expression or repression of the target genes will additionally be depend on a second immune cell specific stimulus, with or without an accessory signal (or “costimulus”).

In one embodiment, the second stimulus will result from activation of an immune cell receptor. Examples of immune cell receptors include T-cell receptors (TCR), B cell receptors (BCR) and Toll-like receptors (TLR). Examples of molecules capable of triggering a TCR or BCR signal include specific antigens for the receptors, superantigens such as TSS1, SEA, SEB, SEC, SED and SEE, antibodies to the TCR αβ chains including Fab, F(ab)2 fragments, phage displayed peptides and ScFV or antibodies to CD3 proteins including ξ and ε chains, anti-CD28 antibodies, anti-BCR antibodies, LPS and other bacterial products, cell receptors involved in phagocytosis such as Fc receptors, complement receptors, mannose receptors and other scavenger receptors, receptors involved in clearance of apoptotic cells such as CD36 and αvβ5, dendritic cell receptors such as DEC205 and DC-light, and activators of TCR and/or BCR signalling pathways such as PMA, ionomycin or kinase inhibitors. These molecules may be used alone or in combination and may be presented on an antigen presenting cell.

In accordance with one embodiment of the present invention there is provided a method for detecting modulators of Hedgehog signalling comprising the steps of:

(a) activating a cell of the immune system;

(b) contacting the cell with a candidate modulator;

(c) monitoring Hedgehog signalling;

(wherein steps (a), (b) and (c) can be carried out in any order); and

(d) determining whether the candidate modulator modulates Hedgehog signalling.

Preferably the activator is an anti-CD3 antibody or an anti-CD28 antibody. In more detail, T-cell activation involves multiple intracellular signaling events originating from the cell surface TCR/CD3 complex. Cross-linking of the TCR/CD3 complex by anti-CD3 antibodies induces T-cell activation, leading to the production of cytokines such as IL-2. IL-2 binds to its high affinity receptor to promote cell proliferation. Additionally co-stimulatory surface molecules such as CD28 have been shown to provide accessory signals in T-cell activation, enhancing IL-2 production, e.g. when combined with an anti-CD3 antibody. CD28 is an antigen expressed on the surface of T-cells, and is also responsible for activation of T-cells.

Accessory or costimulatory signals of immune cell receptor signalling include B7 proteins such as B7.1-CD80, B7.2-CD86, B7H1, B7H2, B7H3, B7RP1, B7RP2, CTLA4, ICOS, CD2, CD24, CD27, CD28, CD30, CD34, CD38, CD40, CD44, CD45, CD49, CD69, CD70, CD95 (Fas), CD134, CD134L, CD153, CD154, 4-1BB, 4-1BB-L, LFA-1, ICAM-1, ICAM-2, ICAM-3, OX40, OX40L, TRANCE/RANK ligands, Fas ligand, MHC class II, DEC205-CD205, CD204-Scavenger receptor, CD14, CD206 (mannose receptor), Toll-like receptors (TLRs), such as TLR 1-9, CD207 (Langerin), CD209 (DC-SIGN), FCγ receptor 2 (CD32), CD64 (FCγ receptor 1), CD68, CD83, CD33, CD54, BDCA-2, BDCA-3, BDCA-4, chemokine receptors, cytokines, growth factors and growth factor receptor agonists, and variants, derivatives, analogues and fragments thereof.

In one embodiment, the second stimulus will be a costimulus. In an alternative embodiment, expression of the target genes will depend on three separate stimuli: Hedgehog signalling, immune cell signalling and a costimulus, all of which are described above. The signals may be delivered all at once or may be phased over a defined period (possibly separated by hours or even days). Preferably, the signals will be delivered substantially simultaneously.

Cell Activation

Immune cell activation may be monitored by any suitable method known to those skilled in the art. For example, cytotoxic activity may be monitored. Natural killer (NK) cells will demonstrate enhanced cytotoxic activity within 4 hours after activation. This cytotoxic activity is maximal after 18 hours.

Once activated, leukocytes express a variety of new cell surface antigens. NK cells, for example, will express transferrin receptor, HLA-DR and the CD25 IL-2 receptor after activation. Activation may therefore be assayed by monitoring expression of these antigens.

Hara et al. Human T-cell Activation: III, Rapid Induction of a Phosphorylated 28 kD/32 kD Disulfidelinked Early Activation Antigen (EA-1) by 12-0-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and continue to appear as late as 48 hours after activation.

These antigens may be useful in detecting leukocyte activation. Additionally, leukocyte activation may be monitored as described in EP 0 325 489 which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognizes a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the activation or stimulation of leukocytes.

Further details of techniques for the monitoring of immune cell activation may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp187-376; ‘Cytokines of the Immune Response’ Chapter 7 in “Handbook of Immune Response Genes”. Mak T. W. and J. J. L. Simard 1998, which are incorporated herein by reference.

Target Genes

The target genes of use in the present invention may be endogenous target genes (i.e. endogenous target genes of the Hedgehog signalling pathway) or synthetic reporter genes. Suitable endogenous target genes of the Hedgehog signalling pathway are discussed above under the sections on the signalling pathways.

In an alternative embodiment of the present invention, the target gene is a reporter gene. In a preferred embodiment, the reporter gene is under the transcriptional control of a promoter region or responder element(s) sensitive to Hedgehog signalling.

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Other protocols include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes may even be used. These and other assays are described, among other places, in Hampton R et al (1990, Serological Methods, A Laboratory Manual, APS Press, St Paul Mn.) and Maddox D E et al (1983) J Exp Med 15(8):121-1.

One skilled in the art will recognize that the identity of the specific reporter gene can, of course, vary. Examples of reporter genes that have been used in the art include, but are not limited to, genes encoding an enzymatic activity such as chloramphenicol acetyltransferase (CAT) gene, Green Fluorescent Protein (GFP), luciferase (luc), β-galactosidase, invertase, horseradish peroxidase, glucuronidase, exo-glucanase, glucoamylase or alkaline phosphatase. Alternatively, the reporter gene may comprise a radiolabel or a fluorescent label such as FITC, rhodamine, lanthanide phosphors, or a green fluorescent fusion protein (See for example Stauber et al (1995) Virol. 213:439-449). Alternatively, the reporter may comprise a predetermined polypeptide epitope which can be recognized by a secondary reporter such as leucine zipper pair sequences, binding sites for secondary antibodies, metal binding domains, or epitope tags. One skilled in the art will appreciate that the specific reporter gene or genes utilized in the methods disclosed herein may vary and may also depend on the specific model system utilized, and the methods disclosed herein are not limited to any specific reporter gene or genes.

By way of further examples, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

The reporter gene used in the method of the present invention is under the transcriptional control of at least one Hedgehog signalling sensitive promoter region and/or responder element. Promoter regions and/or responder elements sensitive to Hedgehog signalling include the regulatory elements of endogenous Hedgehog target genes such as the HES promoters, Deltex promoter, Hedgehog and Hedgehog ligand promoters, IL-10 promoters. Regulatory elements of use in the present invention also include single or multimerized CBF1 sites, CTLA4 promoters and AIRE promoters. The regulatory elements are positioned such that activation of the Hedgehog signalling pathway results in increased expression of the reporter gene.

One or more copies of the reporter gene can be inserted into the hosT-cell by methods known in the art. The term “hosT-cell”—in relation to the present invention includes any cell that could comprise the target for the agent of the present invention. Polynucleotides may be introduced into prokaryotic cells or eukaryotic cells, for example yeast, insect or mammalian cells. Preferably, the hosT-cell will be a cell of the immune system as described above.

Polynucleotides of the invention may be introduced into suitable hosT-cells using a variety of techniques known in the art, such as transfection, transformation and electroporation. Where polynucleotides of the invention are to be administered to animals, several techniques are known in the art, for example infection with recombinant viral vectors such as retroviruses, herpes simplex viruses and adenoviruses, direct injection of nucleic acids and biolistic transformation.

In the present invention, the hosT-cells will preferably be mammalian cells and the polypeptides will be expressed either intracellularly, on the cell membranes or secreted in a culture media if preceded by an appropriate leader sequence.

Expression of the target genes (whether endogenous or synthetic reporter genes) may be dependent on Hedgehog signalling alone or on Hedgehog signalling and one or more further stimulatory signals.

Therapeutic Uses

The present invention is useful in the treatment and/or prevention of a disease. In general, the present invention is useful in the treatment and/or disease which is mediated by T-cells, for example disease which are established or maintained by an inappropriate or excessive T-cell response.

Diseased or infectious states that may be described as being mediated by T-cells include, but are not limited to, any one or more of asthma, allergy, graft rejection, autoimmunity, tumour induced abberrations to the T-cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T-cells include MS, RA and diabetes. The present invention may also be used in organ transplantation or bone marrow transplantation.

The present invention is likely to be particularly useful in the treatment of hypersensitivity disorders. Hypersensitivity reactions include:

(i) allergies, resulting from inappropriate responses to innocuous foreign substances;

(ii) autoimmune diseases, resulting from responses to self tissue antigens; and

(iii) graft rejection, resulting from responses to a transplant.

Examples of allergies include, but are not limited to: hay fever, extrinsic asthma, insect bite and sting allergies, food and drug allergies, allergic rhinitis, bronchial asthma chronic bronchitis, anaphylactic syndrome, urticaria, angioedema, atopic dermatitis, allergic contact dermatitis, erythema nodosum, erythema multiforme, Stevens-Johnson Syndrome, rhinoconjunctivitis, conjunctivitis, cutaneous necrotizing venulitis, inflammatory lung disease and bullous skin diseases.

Examples of the autoimmune diseases include, but are not limited to: rheumatoid arthritis (RA), myasthenia gravis (MG), multiple sclerosis (MS), systemic lupus erythematosus (SLE), autoimmune thyroiditis (Hashimoto's thyroiditis), Graves'disease, inflammatory bowel disease, autoimmune uveoretinitis, polymyositis and certain types of diabetes, systemic vasculitis, polymyositis-dermatomyositis, systemic sclerosis (scleroderma), Sjogren's Syndrome, ankylosing spondylitis and related spondyloarthropathies, rheumatic fever, hypersensitivity pneumonitis, allergic bronchopulmonary aspergillosis, inorganic dust pneumoconioses, sarcoidosis, autoimmune hemolytic anemia, immunological platelet disorders, cryopathies such as cryofibrinogenemia and autoimmune polyendocrinopathies.

A variety of tissues are commonly transplanted in clinical medicine, including kidney, liver, heart lung, skin, cornea and bone marrow. All grafts except corneal and some bone marrow grafts usually require long-term immunosuppression at present.

In one embodiment of this aspect of the invention, the peptide is for use in the treatment and/or prevention of diabetes.

“Autoimmune disease” is used in accordance with its ordinary signification in the art, namely to refer to a disease or component of a disease in which the immune system plays a damaging role by attacking “self” targets. Examples of autoimmune diseases include multiple sclerosis, arthritis and inflammatory bowel disease.

Particular areas of interest include the treatment of immune-related disorders such as organ transplant rejection and autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions, in particular reaction associated with histamine production, and asthma.

The selected antibodies or binding proteins thereof of the present invention will typically find use in preventing, suppressing or treating inflammatory states, allergic hypersensitivity, cancer, bacterial or viral infection, and autoimmune disorders (which include, but are not limited to, Type I diabetes, multiple sclerosis, rheumatoid arthritis, systemic lupus erythematosus (SLE), Crohn's disease and myasthenia gravis).

In a further embodiment of this aspect of the invention, the peptide is for use in the treatment and/or prevention of multiple sclerosis (MS). Multiple sclerosis (MS) is a chronic inflammatory disease characterised by multiple demyelinating lesions disseminated throughout the CNS white matter and occurring at various sites and times (McFarlin and McFarland, 1982 New England J. Medicine 307:1183-1188 and 1246-1251). MS is thought to be mediated by autoreactive T-cells.

The Hedgehog signalling pathway appears to play a role in the development of diseases relates to decreased or increased apoptosis. Diseases arising from decreased apoptosis include, but are not limited to, cancer of the breast, prostate and ovary as well as lymphomas and carcinomas, autoimmune diseases such as systemic lupus erythematosus, glomerulonephritis, Sjogren's syndrome, Graves disease, MS, RA and diabetes, inflammatory diseases such as osteoarthritis, Crohn's disease, inflammatory bowel disease and colitis, proliferative disorders such as atherosclerosis, restenosis, psoriasis, lymphadenopathy, and viral infections such as by herpesviruses, poxviruses and adenoviruses.

Diseases associated with increased apoptosis include, but are not limited to, AIDS and other infectious or genetic immunodeficiencies, neurodegenerative diseases such as Alzheimer's disease, Parkinson's disease, amyotrophic lateral sclerosis, retinitis pigmentosa and cerebellar degeneration, myelodysplastic syndromes such as aplastic anemia, ischemic injuries such as myocardial infarction, stroke and reperfuision injury, toxin-induced diseases such as alcohol-induced liver damage, cirrhosis and lathyrism, wasting diseases such as cachexia, viral infections such as hepatitis B and C, and osteoporosis.

As used herein, the term “apoptosis” refers to a genetically programmed cell death which is regulated throughout the lifetime of an organism. In apoptosis, a triggering agent from either outside or inside the cell causes “cell-suicide” genes to produce enzymes that damage the cell in several ways, including disrupting its cytoskeleton and nucleus. As a result, the cell shrinks and pulls away from neighbouring cells. The DNA within the nucleus fragments, and the cytoplasm shrinks, although the plasma membrane remains intact. Phagocytes in the vicinity then ingest the dying cell. Apoptosis may be regarded as a normal type of cell death and contrasts with necrosis which is a pathological type of cell death that results from tissue injury. Apoptosis removes unneeded cells during development before birth. It continues to occur after birth to regulate the number of cells in a tissue and eliminate potentially dangerous cells such as cancer cells.

For example, the control of apoptosis in neurons may be useful in the treatment of a number of diseases, including but not limited to atherosclerosis, inflammatory conditions, systemic inflammatory response syndrome (SIRS), neurodegenerative diseases, retinal diseases, cancer metastasis, Alzheimer's and Parkinson's disease, adult respiratory distress syndrome (ARDS) and other related conditions, stroke, myocardial infarction, myelosuppression following chemotherapy or irradiation and a significant number of other diseases where cell death is a key feature of the pathology.

If a successful therapeutic outcome is to be achieved, an immunotherapeutic approach to cancer treatment depends on a number of factors. These include the ability to elicit a cytotoxic T-lymphocyte (CTL) response, the ability to elicit an antibody response and, importantly, the ability to break immune tolerance in a subject. The present invention is useful in eliciting an immunotherapeutic anti-tumour response. Advantageously, the response is an anti-tumour immunotherapeutic response which is effective to inhibit, arrest or reverse the development of a tumour in a subject. Advantageously, the present invention is capable of breaking immune tolerance to 5T4 in a subject.

The present invention is therefore also useful in cancer therapy, e.g. the present invention is useful in relation to adenocarcinomas such as: small cell lung cancer, and cancer of the kidney, uterus, prostrate, bladder, ovary, colon and breast.

A disease or disorder may be both associated with both a disregulation in apoptosis and mediated by T-cells.

We have now found that the use of modulators of Hedgehog signalling may prevent and/or promote regression of the above-mentioned diseases.

We also provide a method of treatment for the following diseases/disorders through modulation of the Hedgehog signalling pathway or a pathway which is a target of the Hedgehog signalling pathway:

The present invention is also useful in treating immune disorders such as autoimmune diseases or graft rejection such as allograft rejection.

Examples of disorders that may be treated include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail: Organ-specific autoimmune diseases include multiple sclerosis, insulin dependent diabetes mellitus, several forms of anemia (aplastic, hemolytic), autoimmune hepatitis, thyroiditis, insulitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, GianT-cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, liver cirrhosis or other hepatic diseases, thyroiditis or other glandular diseases, glomeruloneplritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis piginentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

We have found that the use of antagonists, and additionally agonists, of Hedgehog signalling may prevent and/or promote regression of the above-mentioned diseases.

A disease or disorder may be both associated with both a disregulation in apoptosis and mediated by T-cells.

We have now found that the use of modulators of Hedgehog signalling may prevent and/or promote regression of the above-mentioned diseases.

Vectors, hosT-Cells, Expression

The present invention also relates to vectors which comprise a polynucleotide useful in the present invention, hosT-cells which are genetically engineered with vectors of the invention and the production of polypeptides useful in the present invention by such techniques.

For recombinant production, hosT-cells can be genetically engineered to incorporate expression systems or polynucleotides of the invention. Introduction of a polynucleotide into the hosT-cell can be effected by methods described in many standard laboratory manuals, such as Davis et al and Sambrook et al, such as calcium phosphate transfection, DEAE-dextran mediated transfection, transvection, microinjection, cationic lipid-mediated transfection, electroporation, transduction, scrape loading, ballistic introduction and infection.

Representative examples of appropriate hosts include bacterial cells, such as streptococci, staphylococci, E. Coli, streptomyces and Bacillus subtilis cells; fungal cells, such as yeasT-cells and Aspergillus cells; insecT-cells such as Drosophila S2 and Spodoptera Sf9 cells; animal cells such as CHO, COS, HeLa, C127, 3T3, BHK, 293 and Bowes melanoma cells; and planT-cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the invention can be recovered and purified from recombinanT-cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Methods of Delivery

In the present invention the polynucleotide may be delivered to a targeT-cell population, either ex vivo or in vivo, by any suitable Gene Delivery Vehicle.

This includes but is not restricted to, DNA, formulated in lipid or protein complexes or administered as naked DNA via injection or biolistic delivery, viruses such as retroviruses, adenoviruses, herpes viruses, vaccinia viruses, adeno associated viruses. The GDV can be designed by a person ordinarily skilled in the art of recombinant DNA technology and gene expression to express the fusion protein at appropriate levels and with the cellular specificity demanded by a particular application.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a targeT-cell. Optionally, once within the targeT-cell, the vector may then serve to maintain the heterologous DNA within the cell or may act as a unit of DNA replication. Examples of vectors used in recombinant DNA techniques include plasmids, chromosomes, artificial chromosomes or viruses.

The vector can be delivered by viral or non-viral techniques.

Non-viral delivery systems include but are not limited to DNA transfection methods. Here, transfection includes a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), multivalent cations such as spermine, cationic lipids or polylysine, 1,2,-bis (oleoyloxy)-3-(trimethylammonio) propane (DOTAP)-cholesterol complexes (Wolff and Trubetskoy 1998 Nature Biotechnology 16: 421) and combinations thereof.

Viral delivery systems include but are not limited to adenovirus vector, an adeno-associated viral (AAV) vector, a herpes viral vector, a retroviral vector, a lentiviral vector or a baculoviral vector.

Examples of retroviruses include but are not limited to: murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV).

A detailed list of retroviruses may be found in Coffin et al (“Retroviruses” 1997 Cold Spring Harbour Laboratory Press Eds: J M Coffin, S M Hughes, H E Varmus pp 758-763).

Adenoviruses and adeno-associated viruses which have good specificity for epithelial cells are particularly preferred.

Other examples of vectors include ex vivo delivery systems, which include but are not limited to DNA transfection methods such as electroporation, DNA biolistics, lipid-mediated transfection, compacted DNA-mediated transfection.

Thus, nucleic acid vectors according to the invention may be capable of delivery preferentially to the targeT-cell. For example in the case of a retroviral vector, the retroviral envelope protein may be capable of directing the vector to a particular cell type or cell types. For that purpose, the envelope protein may be a modified envelope protein adapted to have a specific targeting ability, or it may be a selected envelope protein derived from a different viral or retroviral source and having the desired targeting ability.

Preferably, the nucleic acid in a vector according to the invention is operatively linked to an expression control sequence capable of causing preferential expression of the fusion protein in the targeT-cell. The expression control sequence may be for example a promotor or enhancer which is preferentially active in certain cell types including the targeT-cell, or a promotor or enhancer which is preferentially active under certain conditions.

The term “promoter” is used in the normal sense of the art, e.g. an RNA polymerase binding site in the Jacob-Monod theory of gene expression.

The term “enhancer” includes a DNA sequence which binds to other protein components of the transcription initiation complex and thus facilitates the initiation of transcription directed by its associated promoter.

Preferably the promoters of the present invention are tissue specific. That is, they are capable of driving transcription of a nucleic acid in one tissue while remaining largely “silent” in other tissue types. A particularly preferred promoter is the epithelial cell promoter.

The term “tissue specific” means a promoter which is not restricted in activity to a single tissue type but which nevertheless shows selectivity in that they may be active in one group of tissues and less active or silent in another group.

Administration

Compounds capable of affecting a component of the Hedgehog family signalling pathway or a target pathway thereof for use in therapy are typically formulated for administration to patients with a pharmaceutically acceptable carrier or diluent to produce a pharmaceutical composition. The formulation will depend upon the nature of the compound identified and the route of administration but typically they can be formulated for local, systemic, oral, topical, parenteral, intramuscular, intravenous, intra-peritoneal, intranasal inhalation, lung inhalation, mucosal, intradermal or intra-articular administration. The compound may be used in an injectable form. It may therefore be mixed with any vehicle which is pharmaceutically acceptable for an injectable formulation, preferably for a direct injection at the site to be treated, although it may be administered systemically.

The pharmaceutically acceptable carrier or diluent may be, for example, sterile isotonic saline solutions, or other isotonic solutions such as phosphate-buffered saline. The compounds of the present invention may be admixed with any suitable binder(s), lubricant(s), suspending agent(s), coating agent(s), solubilising agent(s). It is also preferred to formulate the compound in an orally active form.

In general, a therapeutically effective daily oral or intravenous dose of the compounds of the invention, including compounds of formula (1) and their salts, is likely to range from 0.01 to 50 mg/kg body weight of the subject to be treated, preferably 0.1 to 20 mg/kg. The compounds of the formula (I) and their salts may also be administered by intravenous infusion, at a dose which is likely to range from 0.001-10 mg/kg/hr.

Tablets or capsules of the compounds may be administered singly or two or more at a time, as appropriate. It is also possible to administer the compounds in sustained release formulations.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

Alternatively, the compounds of the invention can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavouring or colouring agents.

The compositions (as well as the compounds alone) can also be injected parenterally, for example intracavernosally, intravenously, intramuscularly or subcutaneously. In this case, the compositions will comprise a suitable carrier or diluent.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

For oral, parenteral, buccal and sublingual administration to subjects (such as patients), the daily dosage level of the compounds of the present invention and their pharmaceutically acceptable salts and solvates may typically be from 10 to 500 mg (in single or divided doses). Thus, and by way of example, tablets or capsules may contain from 5 to 100 mg of active compound for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

In one embodiment, the modulator of Hedgehog signalling may be used as an agonist, which may be used, for example, to achieve an immune stimulating or adjuvant-type effect.

When used as an adjuvant, modulators of the Hedgehog signalling pathway may, for example, be used in vaccine compositions and preparations which may be used to protect or treat a mammal susceptible to, or suffering from disease, eg by administering vaccine via a mucosal route, such as the oralibucal/intestinal/vaginal/rectal or nasal route.

Thus in a further embodiment the invention provides a vaccine composition comprising a modulator of the Hedgehog signalling pathway.

Hedgehog modulators may also be used to enhance the immunogenicity of antigens applied to the skin, for example by intradermal, transdermal or transcutaneous delivery. In addition, such adjuvants may be parenterally delivered, for example by intramuscular or subcutaneous administration.

For certain vaccine formulations, other vaccine components may be included in the formulation. For example the adjuvant formulations of the present invention may also comprise a bile acid or derivative of cholic acid. Suitably the derivative of cholic acid is a salt thereof, for example a sodium salt thereof. Examples of bile acids include cholic acid itself, deoxycholic acid, chenodeoxy colic acid, lithocholic acid, taurodeoxycholate ursodeoxycholic acid, hyodeoxycholic acid and derivatives like glyco-, tauro-, amidopropyl-1-propanesulfonic- and amidopropyl-2-hydroxy-1-propanesulfonic-derivatives of the above bile acids, or N,N-bis (3DGluconoamidopropyl) deoxycholamide.

Suitably, an adjuvant formulation of the present invention may be in the form of an aqueous solution or a suspension of non-vesicular forms. Such formulations are convenient to manufacture, and also to sterilise (for example by terminal filtration through a 450 or 220 nm pore membrane).

Suitably, the route of administration to said host is via the skin, intramuscular or via a mucosal surface such as the nasal mucosa. When the admixture is administered via the nasal mucosa, the admixture may for example be administered as a spray. The methods to enhance an immune response may be either a priming or boosting dose of the vaccine.

The term “adjuvant” as used herein includes an agent having the ability to enhance the immune response of a vertebrate subject's immune system to an antigen.

The term “immune response” includes any response to an antigen or antigenic determinant by the immune system of a subject. Immune responses include for example humoral immune responses (e. g. production of antigen-specific antibodies) and cell-mediated immune responses (e. g. lymphocyte proliferation).

The term “cell-mediated immune response” includes the immunological defence provided by lymphocytes, such as the defence provided by T-cell lymphocytes when they come into close proximity with their victim cells.

When “lymphocyte proliferation” is measured, the ability of lymphocytes to proliferate in response to specific antigen may be measured. Lymphocyte proliferation includes B cell, T-helper cell or CTL cell proliferation.

Compositions of the present invention may be used to formulate vaccines containing antigens derived from a wide variety of sources. For example, antigens may include human, bacterial, or viral nucleic acid, pathogen derived antigen or antigenic preparations, host-derived antigens, including GnRH and IgE peptides, recombinantly produced protein or peptides, and chimeric fusion proteins.

Preferably the vaccine formulations of the present invention contain an antigen or antigenic composition capable of eliciting an immune response against a human pathogen. The antigen or antigens may, for example, be peptides/proteins, polysaccharides and lipids and may be derived from pathogens such as viruses, bacteria and parasites/fungi as follows:

Viral antigens

Viral antigens may be derived, for example, from:

Cytomegalovirus ( especially Human, such as gB or derivatives thereof); Epstein Barr virus (such as gp350); flaviviruses (e. g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992,10, 915-920) or purified or recombinant proteins thereof, such as NP, NA, HA, or M proteins); measles virus; mumps virus; parainfluenza virus; Respiratory Syncytial virus (such as F and G proteins); rotavirus (including live attenuated viruses); Varicella Zoster Virus (such as gpI, II and IE63); and Human Papilloma Virus (HPV) considered to be responsible for genital warts, (HPV 6 or HPV 11 and others), and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Bacterial Antigens

Bacterial antigens may be derived, for example, from:

Bacillus spp., including B. anthracis (eg botulinum toxin); Bordetella spp, including B. pertussis (for example pertactin, pertussis toxin, filamenteous hemagglutinin, adenylate cyclase, fimbriae); Borrelia spp., including B. burgdorferi (eg OspA, OspC, DbpA, DbpB), B. garinii (eg OspA, OspC, DbpA, DbpB), B. afzelii (eg OspA, OspC, DbpA, DbpB), B. andersonii (eg OspA, OspC, DbpA, DbpB), B. hermsii; Campylobacter spp, including C. jejuni (for example toxins, adhesins and invasins) and C. coli; Chlamydia spp., including C. trachomatis (eg MOMP, heparin-binding proteins), C. pneumonie (eg MOMP, heparin-binding proteins), C. psittaci; Clostridium spp., including C. tetani (such as tetanus toxin), C. botulinum (for example botulinum toxin), C. difficile (eg clostridium toxins A or B); Corynebacterium spp., including C. diphtheriae (eg diphtheria toxin); Ehrlichia spp., including E. equi and the agent of the Human Granulocytic Ehrlichiosis; Rickettsia spp, including R.rickettsii; Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (eg PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa; Legionella spp, including L. pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example W097/41731)); Mycobacterium spp., including M. tuberculosis (for example ESAT6, Antigen 85A, -B or -C), M. bovis, M. Ieprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (eg capsular polysaccharides and conjugates thereof, PsaA, PspA, streptolysin, choline-binding proteins) and the protein antigen Pneumolysin (Biochem Biophys Acta, 1989,67,1007; Rubins et al., Microbial Pathogenesis, 25,337-342), and mutant detoxified derivatives thereof (see for example WO 90/06951; WO 99/03884); Treponema spp., including T. pallidum (eg the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasite/Fungal Antigens

Parasitic/fingal antigens may be derived, for example, from:

Babesia spp., including B. microti; Candida spp., including C. albicans;

Cryptococcus spp., including C. neoformans; Entamoeba spp., including E. histolytica;

Giardia spp., including ;G. lamblia; Leshmania spp., including L. major;

Plasmodium. faciparum (MSP1, AMA1, MSP3, EBA, GLURP, RAP1, RAP2, Sequestrin, PfEMP1, Pf332, LSA1, LSA3, STARP, SALSA, PFEXP1, Pfs25, Pfs28, PFS27/25, Pfs16, Pfs48/45, Pfs230 and their analogues in Plasmodium spp.); Pneumocystis spp., including P. ;carinii; Schisostoma spp., including S. mansoni;

Trichomonas spp., including T. vaginalis; Toxoplasma spp., including T. gondii (for example SAG2, SAG3, Tg34); Trypanosoma spp., including T. cruzi.

The amount of protein in each vaccine dose is selected as an amount which induces an immunoprotective response without significant, adverse side effects in typical recipients. Such amount will vary depending upon which specific immunogen is employed and how it is presented. Typically, it is expected that each dose will comprise 1-1000 pg of protein, preferably 1-500 μg, preferably 1-100 μg, most preferably 1 to 50 μg. After an initial vaccination, subjects may receive one or several booster immunisations suitably spaced.

The vaccines of the present invention may also be administered via the oral route. In such cases the pharmaceutically acceptible excipient may also include alkaline buffers, or enteric capsules or microgranules. The vaccines of the present invention may also be administered by the vaginal route. In such cases, the pharmaceutically acceptable excipients may also include emulsifiers, polymers such as CARBOPOL, and other known stablilisers of vaginal creams and suppositories. The vaccines of the present invention may also be administered by the rectal route. In such cases the excipients may also include waxes and polymers known in the art for forming rectal suppositories.

The formulations of the present invention may be used for both prophylactic and therapeutic purposes. In a further aspect of the present invention there is provided an adjuvant combination and a vaccine as herein described for use in medicine. Vaccine preparation is generally described in New Trends and Developments in Vaccines, edited by Voller et al., University Park Press, Baltimore, Maryland, U. S. A. 1978.

It will be appreciated that the adjuvants of the present invention may further be combined with other adjuvants including, for example: Cholera toxin and its B subunit; E. Coli heat labile enterotoxin LT, its B subunit LTB and detoxified versions thereof such as mLT; immunologically active saponin fractions e. g. Quil A derived from the bark of the South American tree Quillaja Saponaria Molina and derivatives thereof (for example QS21, as described in U.S. Pat. No. 5,057,540); the oligonucleotide adjuvant system CpG (as described in WO 96/02555), especially 5′TCG TCG TTT TGT CGT TTT GTC GTT3 (SEQ ID NO. 9); and Monophosphoryl Lipid A and its non-toxic derivative 3-O-deacylated monophosphoryl lipid A (3D-MPL, as described in GB 2,220,211).

Immune Suppression

Alternatively, the modulator may be used as an antagonist, for example to achieve an immune suppressant effect. When used in this way, it may be advantageous to administer an additional immune suppressant agent, including any agent capable of suppressing the immune system and/or a specific immune response. Examples of such agents include methotrexate, azathioprine, cyclophosphamide, cyclosporin, rapamycin (sirolimus) and FK506 (tacrolimus) and their respective pharmaceutically acceptable salts or derivatives.

The present invention is additionally described by way of the following illustrative, non-limiting Examples, which provide a better understanding of the present invention and of its many advantages.

### EXAMPLES

### Example 1

Mice

C57BL/6J mice were purchased from Harlan Orlac (Bicester, UK) and maintained in the MFAA Animal Unit at the University of Edinburgh. All experiments were performed in accordance with the animal ethics regulations of the Home Office in the United Kingdom.

### Example 2

Antibodies

Functional grade anti-CD3e and anti-CD28 antibodies were purchased from Insight Biotechnology Ltd, Wembley, UK. The neutralising anti-Shh antibody 5E1 (Developmental Studies Hybridoma Bank, Iowa City, USA) and the IgG1 isotype control antibody (cell name P3X63Ag8, ECACC, Wiltshire, UK) were purified from hybridoma supernatants using Protein G colurns (Amersham Pharmacia Biotech, Bucks, UK). Western blotting confirmed that 5E1 but not the isotype control antibody bound to Shh peptide (data not shown). Anti-CD4^{FITC} antibody (BD Biosciences, Heidelberg, Germany) was used at a dilution of 1:100 for FACS staining. Both anti-Shh N-19 (1:40 dilution) and anti-Ptc C-20 (1:60 dilution) antibodies for use in immunocytochemistry were goat polyclonal antibodies (Autogen Bioclear, Wiltshire, UK). Both anti-Shh and anti-Ptc are completely tolerated by use of the relevant peptide (data not shown). The secondary antibody for ICC was a biotinylated rabbit anti-goat antibody (Dako Ltd, Cambridgeshire, UK) used at a dilution of 1:400.

### Example 3

Isolation of CD4⁺ T-Cells

Single cell suspensions from pooled C57BL/6 mouse spleens were applied to negative selection CD4⁺ T-cell columns (R&D systems Europe Ltd, Abingdon, UK) as per manufacturers instructions. Purity was checked using FACS staining with an anti-CD4^{FITC} antibody and this ranged between 88-93%.

### Example 4

Culture of CD4⁺ T-Cells

CD4⁺ T-cells were cultured in RPMI 1640 medium (Life Technologies, Paisley, UK) supplemented with 10% FCS (Life Technologies), 2 mM L-glutamine, (Sigma, Dorset, UK), 20 μg/ml penicillin/streptomycin (Life Technologies) and 50 mM 2-mercaptoethanol (Sigma). Anti-CD3/28 antibody activation was carried out at 2 concentrations, namely ‘sub-optimal’ (anti-CD3 at 0.25 μg/ml and anti-CD28 at 0.1 μg/ml) and ‘optimal’ (anti-CD3 at 1 μg/ml and anti-CD28 at 5 μg/ml). Tissue culture plates (Corning Inc., NY, USA) were coated with the anti-CD3 antibody for 90 min at 37° C. prior to addition of the CD4⁺ T-cells. Recombinant mouse Shh amino-terminal peptide (R&D systems Europe Ltd, Abingdon, UK) was added into cultures at a concentration of 500 ng/ml. 5E1 antibody was used at either 20 μg/ml or 50 μg/ml and the isotype control was used at 20 μg/ml. The concentrations used were based on results of dose response curves (data not shown).

### Example 5

T-Cell Proliferation Assays

The CD4⁺ T-cells were cultured as above in 96-well plates with and without addition of exogenous Shh or 5E1. They were pulsed after 48 hr of anti-CD3/28 activation with 20 μl of ³H-TdR (50 μCi/ml) (Amersham), harvested at 72 hr and read on a betaplate scintillation counter (Wallac UK, Milton Keynes, UK).

### Example 6

Immunocytochemistry

Paraffin sections of mouse lymph node and spleen were de-waxed in xylene and re-hydrated through descending alcohols. Antigen retrieval was carried in a microwave using Vector Antigen Retrieval solution (Vector Laboratories Inc., Burlingame, Calif., USA). After blocking endogenous peroxidase in 3% hydrogen peroxide, the sections were loaded onto a Sequenza (Shandon Scientific Ltd, Cheshire, UK). Non-specific binding was blocked using normal rabbit serum, and endogenous biotin was blocked using the Vector blocking kit according to manufacturer's instructions. The primary and secondary antibodies were applied to the sections for 30 min at room temperature. After washing, Vectastain Elite avidin biotin complex (Vector) was then applied according to kit instructions before addition of the substrate diaminobenzidine (Sigma).

### Example 7

Cell Cycle Analysis

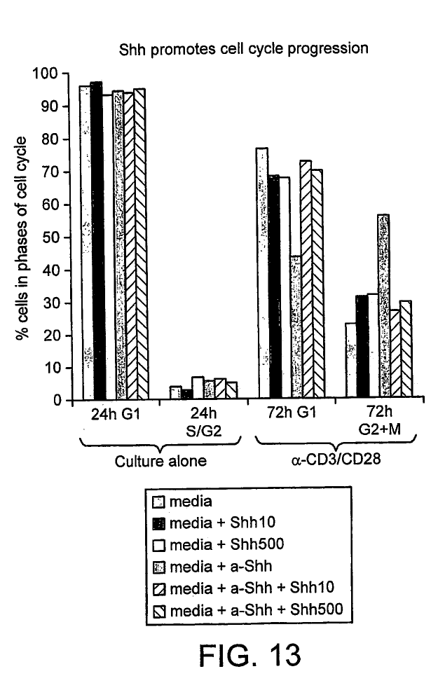

The CD4⁺ T-cells were cultured as above in 48-well plates with and without addition of exogenous Shh or 5E1. At 72 hr post-activation, the cells were spun at 13,000 rpm for 7 min then re-suspended in citrate buffer. Cell cycle analysis was carried out using the Vindelov method (Vindelov et al, 1990). Briefly, the cells were trypsinized (Sigma) to expose the nucleus before being stained with propidium iodide (Sigma). Cell cycle analysis was then performed on an Epics© XL flow cytometer (Beckman Coulter UK Ltd, Bucks, UK). The machine counted 30,000 nuclei in each sample and the software analysed the % of cells in each stage of the cell cycle sub-G1, G1, S phase and G2/M. From these figures, the % of live cells (G1, S and G2/M) was calculated, and from this, the % of live cells in G1 and S/G2 phases. Results of such cell cycle analyses are shown in FIGS. 13, 16, 20 and 24.

FIG. 13 shows the effect of anti-CD3/CD28, of Shh and of anti-Shh neutralising antibody on cell cycle progression. Data at 72 hrs shows that anti-Shh neutralising antibody reduces the percent of cells in G1 and increases those in G2+ M for activated cells. This indicates that Shh protein is being produced by activated T-cells and that this protein is limiting the amount of cell cycle progression of the T-cell population. This effect of anti-Shh was reversed by addition of Shh to the cultures as well. Direct addition of Shh on its own had a slight effect in the opposite direction.

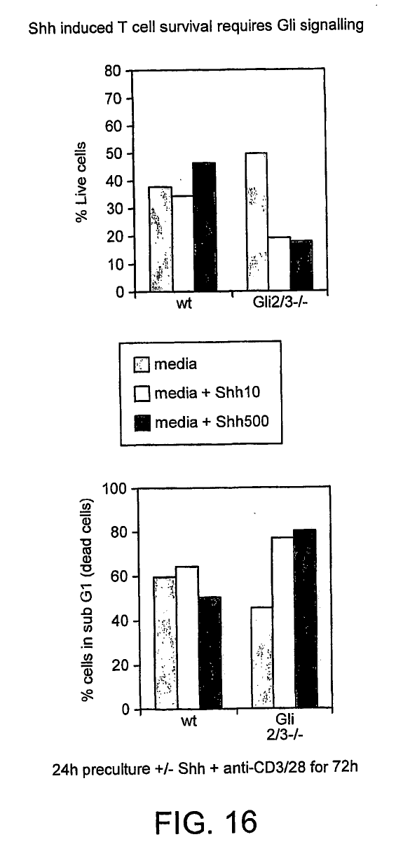

FIG. 16 shows that in Gli2⁻ and Gli3⁻ mice, Shh promotes apoptosis (less live cells and more cells in sub-G1 pool) rather than survival. Clearly Shh can still signal but we can conclude that a partial loss of these two Gli proteins converts the signal from survival promoting to death inducing. This supports to conclusion that Shh works through an appropriate signalling pathway (i.e outcome is influenced by Gli levels).

### Example 8

Statistical Analyses

A paired T test using a one-tailed p value was used to test the significance of differences in ³H-TdR incorporation or % of CD4⁺ T-cells in the proliferative S/G2 phase with and without the addition of Shh or anti-Shh antibody. p values of <0.05 were considered significant.

### Example 9

RNA Isolation

CD4⁺ T-cells were cultured as above in 48 well plates with and without addition of exogenous Shh or 5E1. At various time points (24 hr, 48 hr, 72 hr) post-activation, the CD4⁺ T-cells were spun at 300 g for 7 mins then resuspended in lysis buffer provided as part of the RNeasy kit used for the RNA isolation (Qiagen Ltd, Crawley, UK). Any contaminating DNA was then digested by treating the RNA with DnaseI (Life Technologies) according to the manufacturer's instructions. In order to check that no contaminating DNA remained, a PCR was carried out using genomic β-actin primers (forward primer 5′-CCACCAACTGGGACACATG-3′ (SEQ ID NO:10) and reverse primer 5′-GTCTCAAACATGATCTGGGTCATC-3′ (SEQ ID NO:11)) (MWG-Biotech AG). The PCR program was as follows: 35 cycles of 30 secs at 94° C., 1 min at 58° C., 2 mins at 72° C. followed by a 5 min 72° C. extension then 4° C. hold. This was carried out on a PTC-200 Peltier thermal cycler (MJ Research Inc., Massachusetts, USA).

### Example 10

Reverse Transcription Polymerase Chain Reaction

Reverse transcription of RNA was carried out using M-MLV reverse transcriptase (all components Promega, Southampton, UK). Tubes were incubated at 37° c. for 45 min then 95° c. for 5 min to allow the reverse transcription to take place. The following primer pairs were used for the PCR:

| | | |
|---|---|---|
| Shh fp | AGGGGGTTTGGAAAGAGG | (SEQ ID NO:12) |
| Shh rp | GGATTCATAGTAGACCCAGTCG | (SEQ ID NO:13) |
| ptc fp | ATCGGAGTGGAGTTCACC | (SEQ ID NO:14) |
| ptc rp | CTGCTGTGCTTCGTATTGCC | (SEQ ID NO:15) |
| smo fp | CATCAAGTTCAACAGTTCAGGA | (SEQ ID NO:16) |
| smo rp | ATAGGTGAGGACCACGAACCACACTACTCC | (SEQ ID NO:17) |
| Gli1 fp | GAGAAGCCACACAAGTGC | (SEQ ID NO:18) |
| Gli1 rp | AACAGTCAGTCTGCTCTCTTCC | (SEQ ID NO:19) |

The PCR conditions used were: 35 cycles of 1 min at 94° C., 1 min at 65° C. (60° C. for Shh and Gli1), 2 min at 72° C. followed by an extension of 5 min at 72° C. and a 4° C. hold.

### Example 11

Real Time Polymerase Chain Reaction

Unless otherwise stated all materials for real time PCR were supplied by Applied Biosytems UK, Cheshire, UK. 400 ng of RNA was reverse transcribed using the Multiscribe RT kit. Samples were incubated for 10 min at 25° C., 40mins at 48° C. then 5 min at 95° C. to allow the reverse transcription to take place. cDNA samples were then diluted 1:5 in nuclease-free water (Promega). The PCR step was carried out using Taqman Universal PCR Mastermix, a primer/probe mix specific to the gene of interest and a primer/probe mix specific to 18s rRNA control reagent. The following primer/probe sequences were used:

| | | |
|---|---|---|
| Shh fp | TGACCCCTTTAGCCTACAAGCA | (SEQ ID NO:20) |
| Shh rp | TTCTTGTGATCTTCCCTTCATATCTG | (SEQ ID NO:21) |
| Shh probe | TTTATTCCCAACGTAGCCGAGAAGACCC | (SEQ ID NO:22) |
| ptc fp | CTCCAAGTGTCGTCCGGTTT | (SEQ ID NO:23) |
| ptc rp | TGTACTCCGAGTCGGAGGAATC | (SEQ ID NO:24) |
| ptc probe | CGTGCCTCCTGGTCACACGAACAA | (SEQ ID NO:25) |
| Gli1 fp | GGCTGTCGGAAGTCCTATTCAC | (SEQ ID NO:26) |
| Gli1 rp | CAACCTTCTTGCTCACACATGTAAG | (SEQ ID NO:27) |
| Gli1 probe | CGCACCTTCGGTCGCACACG | (SEQ ID NO:28) |
| bcl-2 fp | GCCCTGTGCCACCATGTG | (SEQ ID NO:29) |
| bcl-2 rp | CGGTAGCGACGAGAGAAGTCA | (SEQ ID NO:30) |
| bcl-2 probe | CCATCTGACCCTCCGCCGGG | (SEQ ID NO:31) |

These probes were all labelled with the fluorescent dye fam (6-carboxy-fluorescein). The primer/probe mix for 18S was supplied by Applied Biosystems which was labelled with the fluorescent dye vic™. Each cDNA sample was run in duplicate 25 μl volumes on a capped 96-well optical reaction plate. The plate was run in the ABI Prism 7700 sequence detector using SDS software. The PCR conditions were as follows: 50° C. for 2 min, 95° C. for 10 min then 40 cycles of 15 sec at 95° C. and 1 min at 60° C. The software then analysed the data and output a pair of ‘ct’ values for each sample. ‘Ct’ is the number of cycles needed to result in a signal crossing a set threshold. Each sample yielded two ct values, one for the gene of interest and one for the 18S house keeping control. The ct values were then transported to a Microsoft Excel spreadsheet and analysed to give a value representing the relative mRNA levels present for the gene of interest linearly as per the manufacturers instructions.

### Example 12

Expression of Hedgehog Signalling Pathway Components in Resting and Activated Peripheral CD4⁺ T-Cells and Secondary Lymphoid Tissue

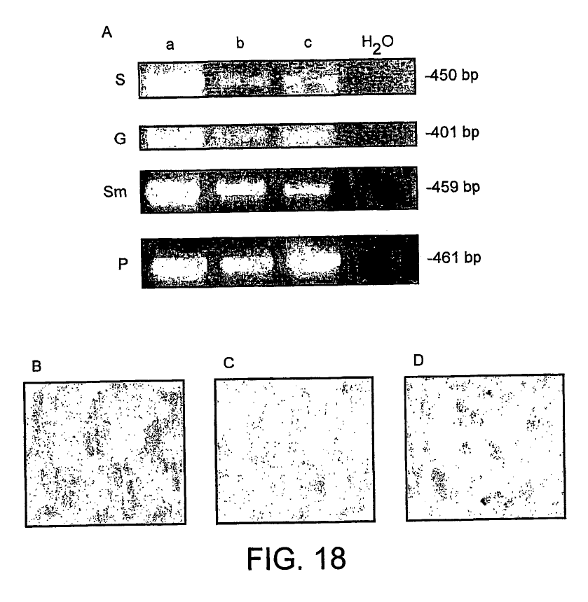

Expression of mRNAs encoding Shh, Ptc, Smo and Gli1 was investigated using RT-PCR. RNA from adult thymus was used as a positive control and was compared to the expression of these genes in both resting (t=0) and anti-CD3/CD28 antibody activated (t=72 hr) CD4⁺ T-cells. Specific transcripts for Shh, Ptc, Smo and Gli1 were detected in both resting and activated CD4⁺ T-cells (FIG. 18).

Members of the Shh signalling pathway are expressed in the thymus (Outram et al). In particular, Shh has been observed on thymic epithelial cells but not thymocytes. By contrast, the receptors Smo and Pct have been detected on thymocytes at various stages of development (Outram et al). Furthermore, transcripts for Shh, ptc and smo have been detected in mature CD3⁺ T-cell populations (15), which is in agreement with the findings reported here.

To verify the presence of components of the hedgehog signalling pathway in the peripheral immune system, expression of the Shh and Ptc proteins was investigated in the spleen and lymph node using immunocytochemistry. Both ptc and Shh expressing cells were present in the spleen (FIGS. 18B-D) and lymph nodes (data not shown).

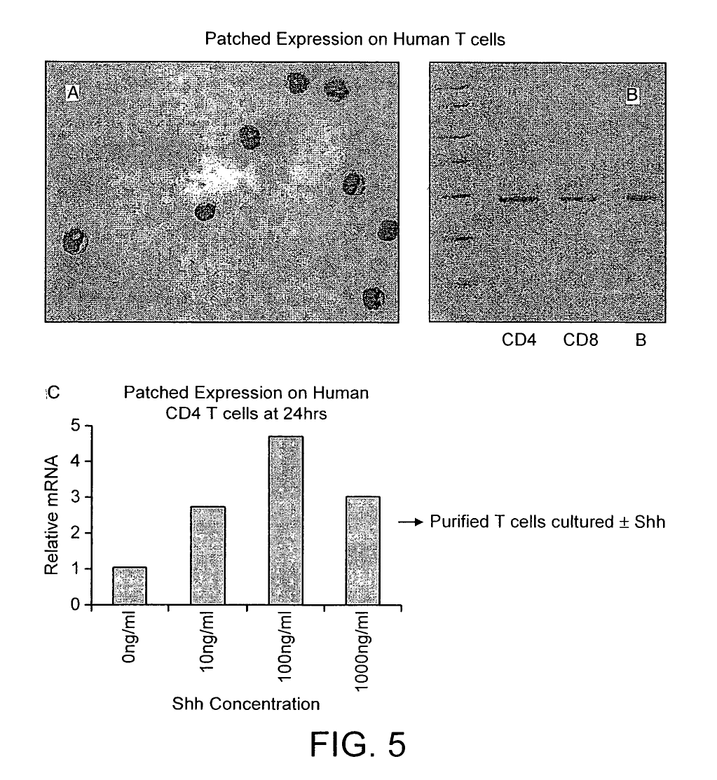

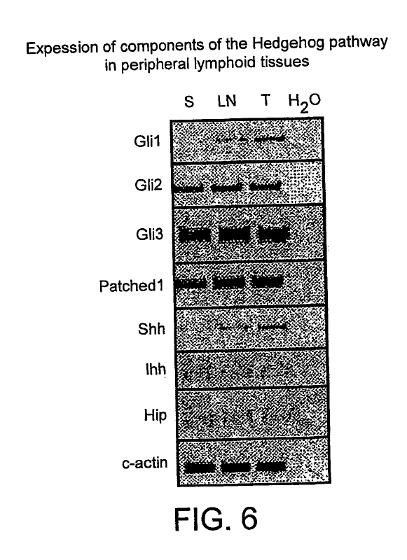

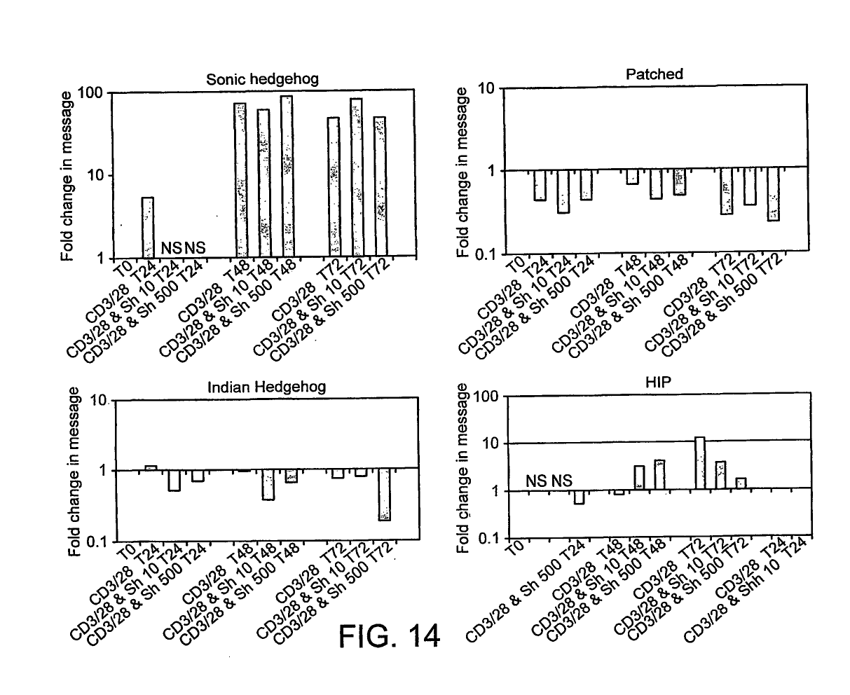

Similar experiments were used to study the expression of Shh (FIG. 4) and Ptc (FIG. 5) in T-cells and the expression of components of the Hh signalling pathway in lymphoid tissues (FIG. 6). FIG. 14 illustrates the results of an RT-PCR analysis of Shh, Ihh, Hip and Ptc expression. Activation of T-cells induced a sustained and marked (approximately 80-fold) increase in Shh mRNA, peaking at 44 and 72 hours. It lead to a downregulation of Ptc. At 24 hours, Shh addition is thought to downregulate Shh mRNA expression as no signal is detected at 40 cycles of PCR (the limit of the assay). Shh does not appear to affect Ptc downregulation. Ihh and Hip are down- and up-regulated, respectively, on addition of Shh to the activated T-cell cultures.

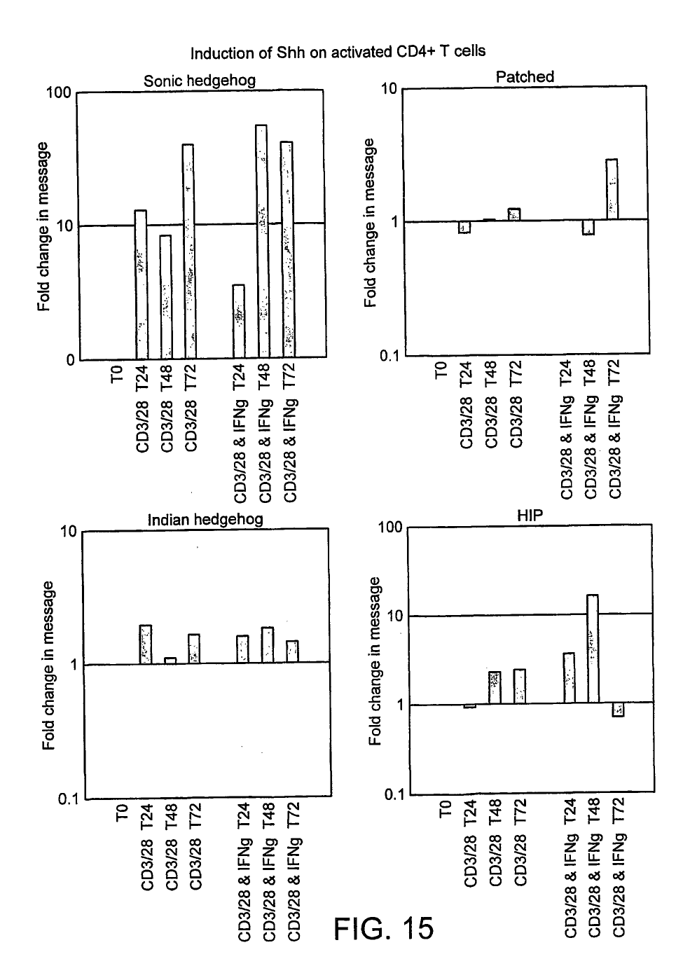

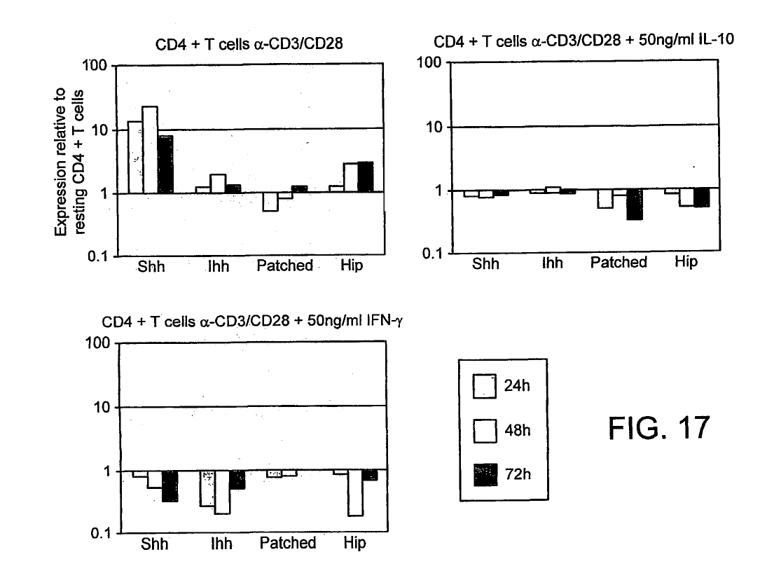

Expression of these genes is also modulated by cytokines (FIGS. 15 and 17). IFNγ modulates Shh expression in activated T-cells (a downregulation is observed at 24 hours (early); an upregulation is observed at 48 hours). Hip was upreguated at 24 and 48 hours but appears to be downregulted at 72 hours. Thus, it would appear that T-cell activation and response to cytokines can be modulated by Shh pathway.

### Example 13

Shh Peptide Promotes Peripheral CD4⁺ T-Cell Proliferation

Purified peripheral CD4⁺ T-cells were cultured with and without the addition of the biologically active amino terminal Shh peptide. An initial titration curve established that 500 ng/ml was the optimal dose of the Shh peptide to enhance proliferation of peripheral CD4⁺ T-cells (data not shown), and this concentration was used in all subsequent experiments. Shh was added to CD4⁺ T-cells that were resting, maximally stimulated with anti-CD3 (1 μg/ml) and anti-CD28 (5 μg/ml) antibodies or sub-optimally activated with anti-CD3 (0.25 μg/ml) and anti-CD28 (0.1 μg/ml).

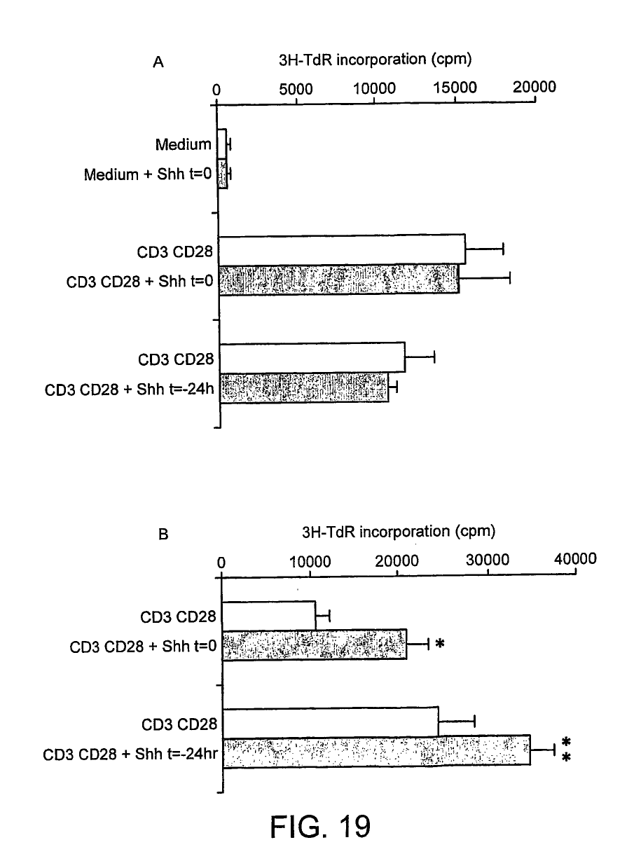

No significant difference in the degree of proliferation was observed following the addition of the Shh peptide in resting CD4⁺ T-cells (FIG. 19A). In maximally stimulated T-cells, the Shh peptide was added at 2 time points, namely 24 hr before (t=-24 hr) or at the time of anti-CD3/28 activation (t=0). However, no significant difference in the level of proliferation as determined by ³[H]-TdR incorporation was detected at either time point (FIG. 19A).

Since Shh appeared to have no modulatory effects on CD4⁺ T-cells that had been maximally stimulated with anti-CD3/28 antibody treatment (NB. It is unlikely that in vivo antigen would be encountered in an environment that would result in the level of activation mediated by saturating doses of anti-CD3 and anti-CD28 antibodies in vitro.), these experiments were repeated in the presence of sub-optimal anti-CD3 (0.25 μg/ml) and anti-CD28 (0.1 μg/ml) stimulation. The Shh peptide was added into culture at t-24 hr and t=0 relative to activation as before. Addition of the Shh peptide at t=0 produced a significant increase in CD4⁺ T-cell proliferation ranging from 76.2-128% (mean=101.6%; p<0.01; FIG. 19B). Proliferation was measured by ³H-TdR incorporation and was determined at 72 hr. Addition of Shh peptide 24 hr prior to sub-optimal anti-CD3/28 activation also produced a significant increase in proliferation ranging from 16-63% (mean=46%; n=3; p<0.04; FIG. 19B).

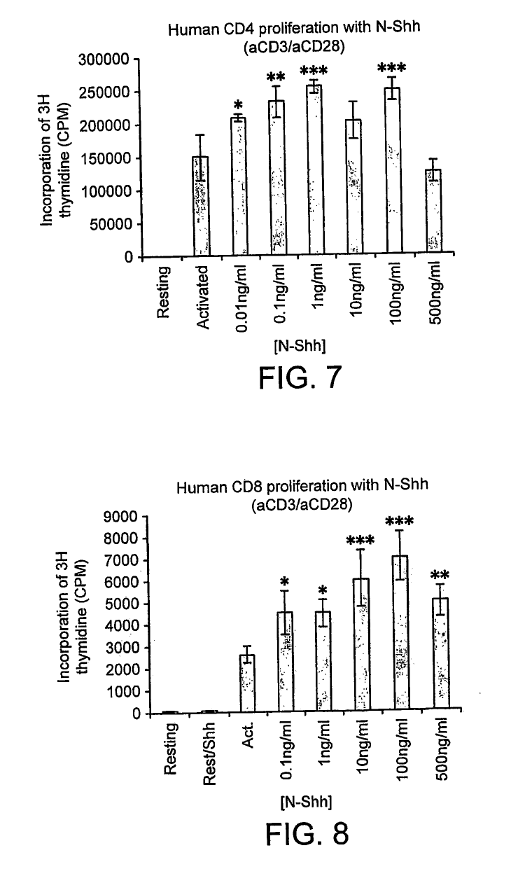

Results from similar experiments are also illustrated in FIGS. 7 and 8. FIGS. 7 and 8, respectively show that Shh significantly enhances the incorporation of 3H-thymidine in activated CD4+ and CD8+ T-cells, in a dose dependent manner. This could reflect either increased proliferation or increased survival (or a combination of both).

Cell proliferation can also be analysed by Trypan cell staining (FIGS. 11 and 12). As shown in FIG. 12, Shh addition to mouse spleen cells and purified CD4+ T-cells in culture enhances their survival over a 4 day time frame.

### Example 14

Shh Peptide Promotes Cell Entry into S/G2 Phase

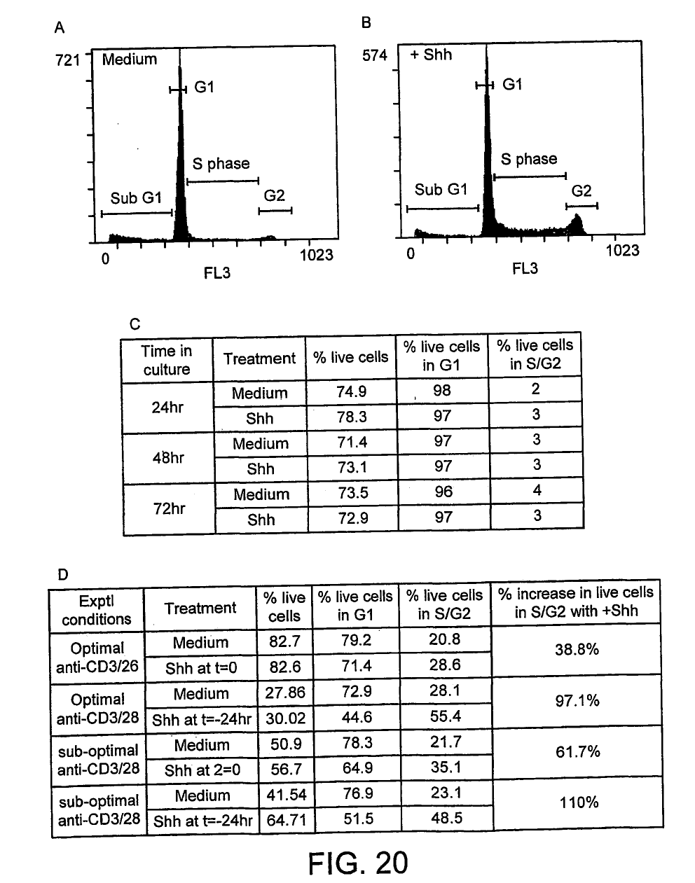

The effect of Shh on CD4⁺ T-cell proliferation was investigated further using cell cycle analysis to allow us to examine if Shh affected cell survival or promoted entry in the S/G2 proliferative phase of the cell cycle. As with the ³H-TdR incorporation studies, this analysis was carried out on resting CD4⁺ T-cells and those optimally and sub-optimally activated. Exogenous Shh was added at the time of (t=0) or 24 hr before (t=−24 hr) anti-CD3/28 antibody activation and the T-cells were analysed 72 hr later. In the case of resting CD4⁺ T-cells, Shh peptide was added at t=0, and the cells were analysed at 24 hr, 48 hr and 72 hrs. The % cells distributed in sub-G1, G1, S and G2 phases of the cell cycle was analysed, and from this the % live cells in G1 and S/G2 phases was calculated. FIG. 20A and 20B shows a representative plot of the cell cycle distribution in the presence or absence of Shh (500 ng/ml) to demonstrate how the cell cycle was analysed.

The addition of Shh to resting CD4⁺ T-cells had minimal effects on cell survival. The % of live, non-activated CD4⁺ T-cells was very similar in cultures with and without Shh added (FIG. 20B). The difference in the % of cells in S /G2 phase was negligible.

In optimally activated CD4⁺ T-cells (anti-CD3=1 μg/ml, anti-CD28=5 μg/ml), the addition of Shh at t=0 promoted CD4⁺ T-cell entry into the S/G2 proliferative phase of the cell cycle. The % of live cells is very similar with and without Shh added, but of those live cells, the addition of exogenous Shh promotes increased proliferation by entry into S/G2 phase (FIG. 20D). However, this % increase in live cells in S/G2 phase showed a variable range from 9%-144% (mean=63.7%; n=3) and did not reach statistical significance. Adding Shh 24 hr prior to optimal activation also promoted CD4⁺ T-cell entry into the S/G2 proliferative phase of the cell cycle. Again, the % of live cells is very similar with and without Shh added, but addition of Shh showed an increase in cells in S/G2 phase which ranged from 53.1%-97.1% (mean=71.5%; n=3; p<0.01).

In order to investigate whether or not this increase in CD4⁺ T-cell proliferation in response to exogenous Shh could be further augmented in the absence of maximal anti-CD3/28 antibody treatment, cell cycle analysis was also performed in sub-optimally activated CD4⁺ T-cells. In these CD4⁺ T-cells, addition of Shh peptide at time=0 also resulted in an increase in proliferation (FIG. 20D). As before with the optimally activated CD4⁺ T-cells, the % live cells is similar with and without addition of Shh, but of those live cells, adding Shh peptide at t=0 promotes cell entry into the proliferative S/G2 phase, with the % increase in live cells in S/G2 phase ranging from 27.8%-77.8% (mean =55.8%; n=3; p<0.02). Addition of the Shh peptide 24 hr prior to sub-optimal activation revealed an increase in the % of live CD4⁺ T-cells (p<0.02). However, as FIG. 20D shows, the pattern of the previous experiments was repeated, as a significantly higher % of those live cells entered the proliferative S/G2 phase with addition of exogenous Shh ranging from 34.6%-110% (mean=61.5%; n=3; p<0.03). Further tests are shown in FIG. 13.

### Example 15

Anti-Shh Antibody Inhibits TCR-Mediated CD4⁺ T-Cell Proliferation in Vitro

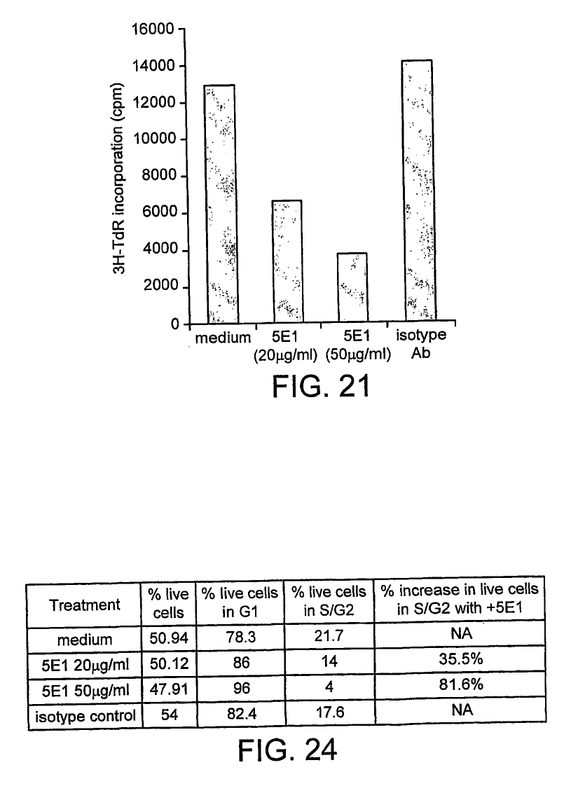

Given that exogenous Shh promotes the proliferation of activated CD4⁺ T-cells, we were prompted to investigate if CD4⁺ T-cells produce Shh following TCR mediated signalling. CD4⁺ T-cells were activated with anti-CD3/CD28 antibodies in the presence of a neutralising anti-Shh antibody (5E1). The sub-optimally activated CD4⁺ T-cells were used in this set of experiments as under these conditions the cells showed increased proliferation as determined by both ³H-TdR incorporation and enhanced entry into the S/G2 phase of the cell cycle. The addition of anti-Shh antibody at the time of activation resulted in dose-dependent inhibition of proliferation. In the presence of 50 μg/ml of anti-Shh antibody, the decrease ranged from 71.3%-85.1% (n=3; p<0.03; FIG. 21). Inhibition of proliferation was not detected in the presence of the isotype control antibody. These results demonstrate that endogenous Shh is produced by activated CD4⁺ T-cells since the neutralising antibody binds to Shh but not to the receptor, ptc.

### Example 16

The Anti-Shh Antibody Blocks Cell Entry into S/G2 Phase

The effect of the anti-Shh antibody on the cell cycle was also investigated (FIG. 24). As with the Shh peptide studies, the anti-Shh antibody does not alter the % live cells in the culture but exerts its effect by blocking the entry of the CD4⁺ T-cells into the proliferative S/G2 phase of the cell cycle. The % decrease in the proportion of CD4⁺ T-cells in S/G2 with addition of the anti-Shh antibody (50 μg/ml), ranged from 66.2% to 81.6% (mean=73.4%; n=3; p<0.02). This effect was not seen with the isotype control antibody.

### Example 17

Kinetic Analysis of Expression of Shh, ptc, Gli1 and bcl-2 in Activated CD4⁺ T-cells in the Presence and Absence of Exogenous Stimulated Shh

In order to analyse the mechanisms of Shh amplification of TCR mediated activation in CD4⁺ T-cells, the kinetics of expression of components of the Shh signalling pathway and bcl-2 were analysed in activated CD4⁺ T-cells in the presence and absence of exogenous Shh. CD4⁺ T-cell cultures were set up as before, sub-optimally activated with anti-CD3/CD28 and Shh was added at t=0. RNA was extracted at 24, 48 and 72 hr post-activation. It has been reported that a 2× or greater increase in the transcription of any gene on at least 2 occasions is considered to be significant (Chtanova et al and Granucci et al). Proliferation assays and cell cycle analyses were also performed concurrently to ensure that the Shh peptide showed enhanced proliferation in these CD4⁺ T-cell cultures.

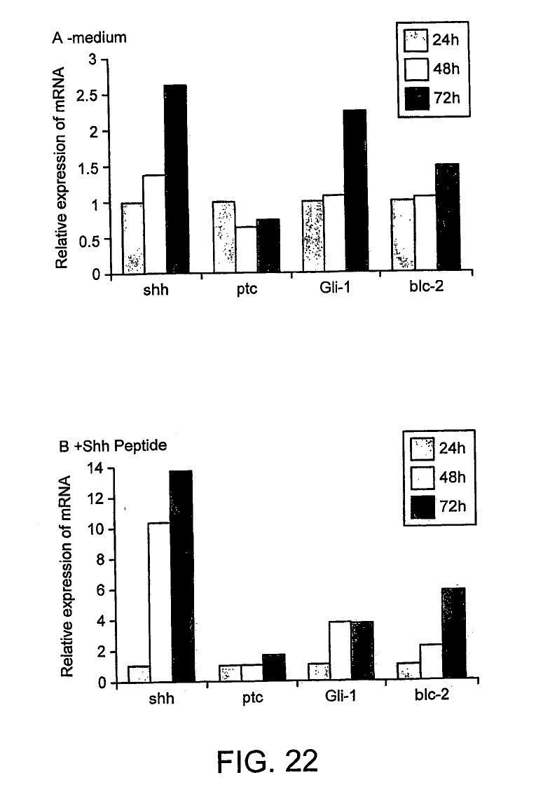

In order to perform a time course analysis, the 48 and 72 hr samples were normalised to the 24 hr RNA sample, assigned a value of 1. In sub-optimally anti-CD3/CD28 activated CD4⁺ T-cells in the absence of Shh we detected a significantly increased transcription of Shh and Gli1, no significant changes were measured for the experiment of either ptc or bcl-2 (FIG. 22A). In the presence of exogenous Shh, Shh transcription was increased both at 48 hr and 72 hr. Gli1 transcription increased at 48 hr and was maintained at 72 hr. Bcl-2 transcripts increased at 48 hr and at 72 hr. However, although ptc transcription was marginally higher at 72 hr it did not reach significance (FIG. 22B).

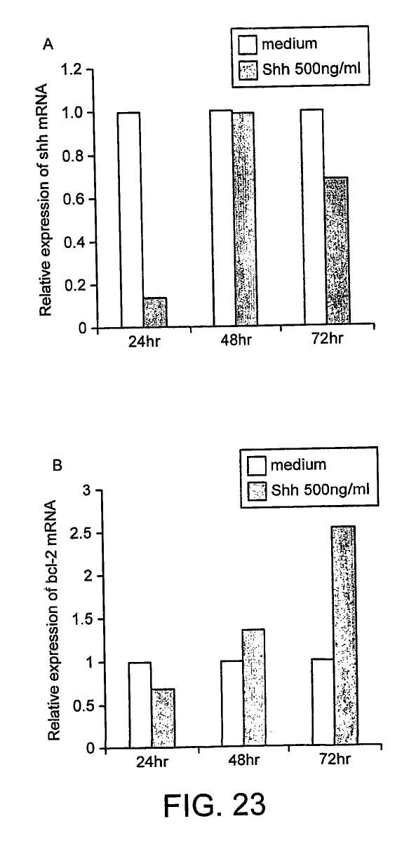

To examine the effect of the Shh peptide on transcription of the various genes, the RNA samples from activated CD4⁺ T-cell cultures with addition of the Shh peptide were normalised against the media only activated cultures at equivalent time points (24 hr, 48 hr and 72 hr). No difference in the level of transcription of ptc or Gli1 was seen between activated CD4⁺ T-cells with and without exogenous Shh peptide throughout the time course. Transcription of Shh was significantly reduced at 24 hr in activated CD4⁺ T-cells with exogenous Shh peptide added compared with media only activated CD4⁺ T-cells (FIG. 23A). Transcription of bcl-2 was significantly increased at 72 hr in activated CD4⁺ T-cells with exogenous Shh peptide added compared with media only activated CD4⁺ T-cells (FIG. 23B).

### Example 18

FACS Analysis

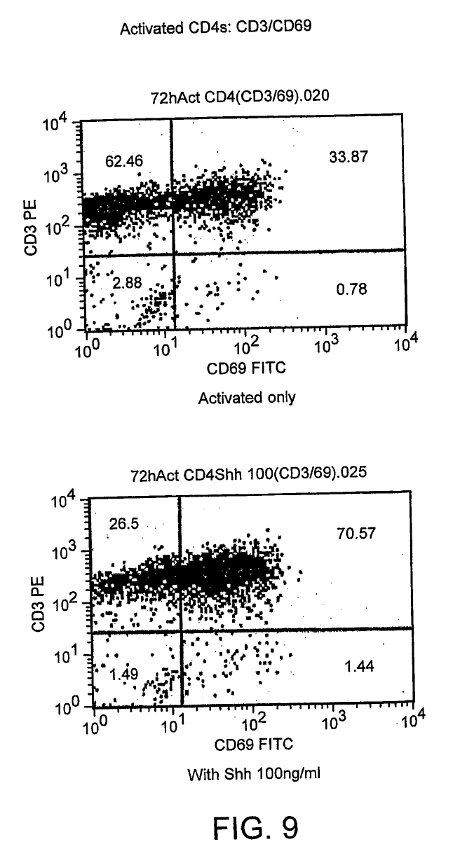

FIG. 9 shows two-colour FACS analysis profiles of CD69 expression in CD3 positive T-cells, in CD4+ T-cells activated for 72 hours with anti-CD3 and anti-CD28 alone (activated only) or with Shh added at 100 ng/ml. FIG. 9 shows that Shh treatment increases CD69 expression from 33.87% to 70.57%.

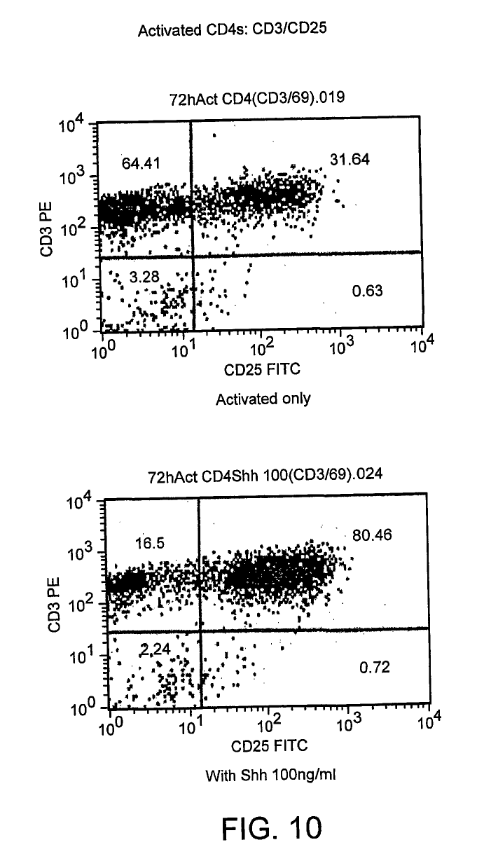

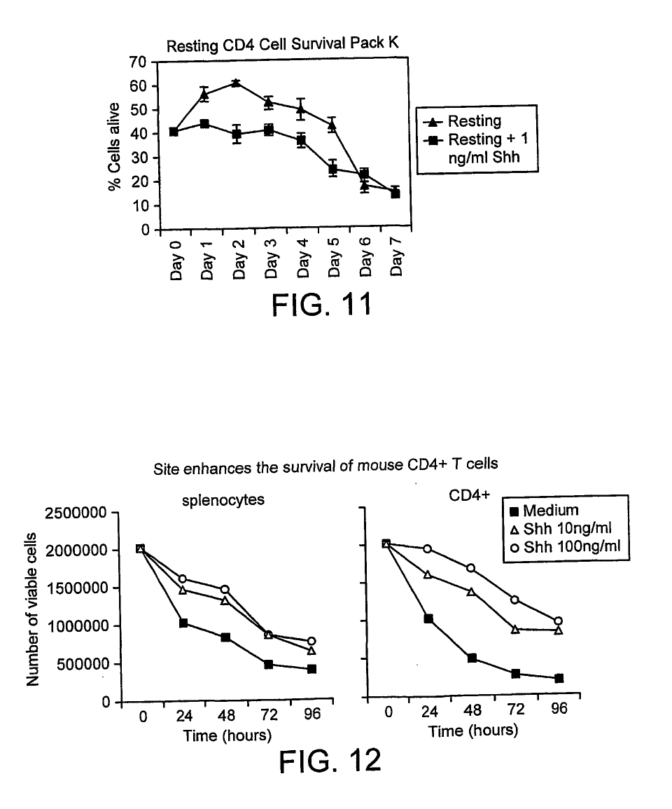

FIG. 10 shows two-colour FACS analysis profiles of CD25 expression in CD3 positive T-cells, in CD4+ T-cells activated for 72 hours with anti-CD3 and anti-CD28 alone (activated only) or with Shh added at 100 ng/ml. FIG. 10 shows that Shh treatment increases CD25 expression from 31.64% to 80.46%.

We have also now shown that IL-10 and TGF-β down-regulate Shh expression.

In summary, the present inventors have surprisingly found that the following effects of Shh on T-cells using human and mouse studies:

Promotes survival of resting T-cells

Regulates cell-cycle entry of activated T-cells

Antibody blockage in activated T-cells modulates cell-cycle entry and this shows that T-cells also make Shh protein.

Upregulates T-cell activation markers such as CD69 and CD25

Modulates expression of Shh and other signalling components as shown by PCR

In Gli 2/3 heterozygous knockouts, Shh effect on T-cells are altered and this indicates that Shh is working through a classical signalling pathway

IL-10, TGF-β and IFN-gamma downregulation of Shh production by activated T-cells

Ab staining immunochemistry shows Shh and Ptc protein are expressed by T-cells

Shh can modulate Ptc expression by T-cells

Shh can modulate T-cell gene expression patterns.

The invention is further described by the following numbered paragraphs:

1. A method of modulating T-cell activation comprising contacting T-cells with a modulator of a Hedgehog signalling pathway or a modulator of a pathway which is a target of the Hedgehog signaling pathway.

2. The method according to paragraph 1, wherein the Hedgehog signalling pathway is the Sonic hedgehog, Indian hedgehog, or Desert hedgehog signalling pathway.

3. The method according to paragraph 1, wherein the pathway which is a target of the Hedgehog signaling pathway is the Wnt signaling pathway.

4. The method according to paragraph 1, wherein the modulator is an inhibitor or upregulator of the biological activity of the pathway.

5. The method according to paragraph 4, wherein the inhibitor is selected from the group consisting of HIP, cyclopamine, Frzb, Cerberus, WIF-1, Xnr-3, Gremlin, Follistatin or a derivative, fragment, variant, mimetic, homologue or analogue thereof, Ptc, Cos2, PKA, and an agent of the cAMP signal transduction pathway.

6. The method according to paragraph 1, wherein the modulator is selected from the group consisting of TGF-β family members, interleukins, IFN-γ, an FLT3 ligand, BMP superfamily members, antibodies, and small organic compounds.

7. The method according to paragraph 6, wherein the TGF-β family members are TGF-β-1 or TGF-β-2.

8. The method according to paragraph 6, wherein the interleukins are IL-4, IL-10, or IL-13.

9. A method of modulating T-cell proliferation comprising contacting T-cells with a modulator of a Hedgehog signalling pathway or a modulator of a pathway which is a target of the Hedgehog signalling pathway.

10. The method according to paragraph 9, wherein the Hedgehog signalling pathway is the Sonic hedgehog, Indian hedgehog, or Desert hedgehog signalling pathway.

11. The method according to paragraph 9, wherein the pathway which is a target of the Hedgehog signaling pathway is the Wnt signaling pathway.

12. The method according to paragraph 9, wherein the modulator is an inhibitor or upregulator of the biological activity of the pathway.

13. The method according to paragraph 12, wherein the inhibitor is selected from the group consisting of HIP, cyclopamine, Frzb, Cerberus, WIF-1, Xnr-3, Gremlin, Follistatin or a derivative, fragment, variant, mimetic, homologue or analogue thereof, Ptc, Cos2, PKA, and an agent of the cAMP signal transduction pathway.

14. The method according to paragraph 9, wherein the modulator is selected from the group consisting of TGF-β family members, interleukins, IFN-γ, an FLT3 ligand, BMP superfamily members, antibodies, and small organic compounds.

15. The method according to paragraph 14, wherein the TGF-β family members are TGF-β-1 or TGF-β-2.

16. The method according to paragraph 14, wherein the interleukins are IL-4, IL-10, or IL-13.

17. A method of modulating T-cell apoptosis comprising contacting T-cells with a modulator of a Hedgehog signalling pathway or a modulator of a pathway which is a target of the Hedgehog signalling pathway.

18. The method according to paragraph 17, wherein the Hedgehog signalling pathway is the Sonic hedgehog, Indian hedgehog, or Desert hedgehog signalling pathway.

19. The method according to paragraph 17, wherein the pathway which is a target of the Hedgehog signaling pathway is the Wnt signaling pathway.

20. The method according to paragraph 17, wherein the modulator is an inhibitor or upregulator of the biological activity of the pathway.

21. The method according to paragraph 20, wherein the inhibitor is selected from the group consisting of HIP, cyclopamine, Frzb, Cerberus, WIF-1, Xnr-3, Gremlin, Follistatin or a derivative, fragment, variant, mimetic, homologue or analogue thereof, Ptc, Cos2, PKA, and an agent of the cAMP signal transduction pathway.

22. The method according to paragraph 17, wherein the modulator is selected from the group consisting of TGF-β family members, interleukins, IFN-γ, an FLT3 ligand, BMP superfamily members, antibodies, and small organic compounds.

23. The method according to paragraph 22, wherein the TGF-β family members are TGF-β-1 or TGF-β-2.

24. The method according to paragraph 22, wherein the interleukins are IL-4, IL-10, or IL-13.

25. A composition for treatment of T-cell mediated diseases comprising a therapeutically effective amount of a modulator of a Hedgehog signalling pathway or a modulator of a target pathway of the Hedgehog signalling pathway and a pharmaceutically acceptable carrier, diluent or excipient.

26. The composition according to paragraph 25 wherein the T-cell mediated diseases are associated with modification of T-cell activation, T-cell proliferation, peripheral T-cell activation, peripheral T-cell proliferation, or T-cell apoptosis.

27. The composition according to paragraph 26, wherein the T-cell mediated diseases are selected from the group consisting of breast cancer, prostate cancer, ovarian cancer, lymphoma, carcinoma, tumour induced abberrations to the T-cell system, autoimmune diseases, inflammatory diseases, proliferative disorders, viral infections, infectious or genetic immunodeficiencies, neurodegenerative diseases, myelodysplastic syndromes, ischemic injuries, toxin-induced diseases, wasting diseases, and infectious diseases.

28. The composition according to paragraph 27 wherein the autoimmune diseases are selected from the group consisting of systemic lupus erythematosus, glomerulonephritis, Sjogren's syndrome, Graves disease, MS, RA, psoriasis, and diabetes.

29. The composition according to paragraph 27, wherein the inflammatory diseases are selected from the group consisting of osteoarthritis, Crohn's disease, inflammatory bowel disease, colitis, allergy, graft rejection, and asthma.

30. The composition according to paragraph 27, wherein the proliferative disorders are selected from the group consisting of atherosclerosis, restenosis, psoriasis, and lymphadenopathy.

31. The composition according to paragraph 27, wherein the viral infections are selected from the group consisting of herpesviruses, poxviruses, adenoviruses, hepatitis B, hepatitis C, Cytomegalovirus, Haemophilus influenza type B, and measles.

32. The composition according to paragraph 27, wherein the infectious immunodeficiency is AIDS.

33. The composition according to paragraph 27, wherein the neurodegenerative diseases are selected from the group consisting of Alzheimer's disease, Parkinson's disease, amyotrophic lateral sclerosis, retinitis pigmentosa, and cerebellar degeneration.

34. The composition according to paragraph 27, wherein the myelodysplastic disease is aplastic anemia.

35. The composition according to paragraph 27, wherein the ischemic injury is selected from the group consisting of myocardial infarction, stroke, and reperfusion injury.

36. The composition according to paragraph 27, wherein the toxin-induced disease is selected from the group consisting of alcohol-induced liver damage, cirrhosism, and lathyrism.

37. The composition according to paragraph 27, wherein the wasting disease is cachexia.

38. The composition according to paragraph 27, wherein the infectious disease is caused by Plasmodium species, Microfilariae, Helminths, Mycobacteria, Pseudomonas, Toxoplasma, Echinococcus, or Toxicara.

39. A method for detecting modulators of Hedgehog signalling comprising the steps of monitoring Hedgehog signalling in a cell of the immune system in the presence and absence of a candidate modulator, and determining whether the candidate modulator modulates Hedgehog signalling.

40. The method according to paragraph 39 wherein the candidate modulator is selected from the group consisting of an organic compound, a inorganic compound, a peptide or polypeptide, a polynucleotide, an antibody, a fragment of an antibody, a cytokine and a fragment of a cytokine.

41. The method according to paragraph 39 wherein the cell of the immune system is a T-cell, a T-cell progenitor, or an antigen presenting cell (APC).

42. The method according to paragraph 39 wherein the step of monitoring Hedgehog signalling comprises monitoring levels of expression of at least one target gene.

43. The method according to paragraph 42 wherein expression of the at least one target gene is monitored with a protein assay.

44. The method according to paragraph 42 wherein expression of the at least one target gene is monitored with a nucleic acid assay.

45. The method according to paragraph 42 wherein the at least one target gene is selected from the group consisting of: an endogenous target gene of Hedgehog signalling, a reporter gene, a gene encoding a polypeptide having an enzymatic activity, a gene comprising a radiolabel, a gene comprising a fluorescent label, and a gene encoding a predetermined polypeptide epitope.

46. The method according to paragraph 42 wherein the at least one target gene is under the transcriptional control of a promoter region sensitive to Hedgehog signalling.

47. The method according to paragraph 42 wherein the at least one target gene is under the transcriptional control of a promoter region sensitive to Hedghog signalling and additionally a second signal; and optionally a third signal, wherein the second and third signals are different.

48. The method according to paragraph 47 wherein the second signal results from activation of a signalling pathway specific to cells of the immune system.

49. The method according to paragraph 48 wherein the signalling pathway specific to cells of the immune system is selected from the group consisting of: a T-cell receptor (TCR) signalling pathway, a B cell receptor (BCR) signalling pathway, and a Toll-like receptor (TLR) signalling pathway.

50. The method according to paragraph 47 wherein the third signal is a costimulus specific to cells of the immune system.

51. The method according to paragraph 50 wherein the costimulus is selected from the group consisting of: B7 proteins B7.1-CD80, B7.2-CD86, B7H1, B7H2, B7H3, B7RP1, B7RP2, CTLA4, ICOS, CD2, CD24, CD27, CD27L, CD3, CD30, CD30L, CD34, CD38, CD40, CD40L, CD44, CD45, CD49, CD69, CD70, CD95 (Fas), CD134, CD134L, CD153, CD154, 4-1BB, 4-1BB-L, LFA-1, ICAM-1, ICAM-2, ICAM-3, OX40, OX40L, PD-1, PDL1, PDL2, TIM-1, TRANCE/RANK ligands, Fas ligand, MHC class II, DEC205-CD205, CD204-Scavenger receptor, CD14, CD206 (mannose receptor), Toll-like receptors (TLRs), such as TLR 1-11, CD207 (Langerin), CD209 (DC-SIGN), FCy receptor 2 (CD32), CD64 (FC-γ receptor 1), CD68, CD83, CD33, CD54, BDCA-2, BDCA-3, BDCA-4, chemokine receptors, cytokines, growth factors and growth factor receptor agonists, and variants, derivatives, analogues and fragments thereof.

52. A modulator identifiable by the method according to paragraph 39.

53. A method for detecting modulators of Hedgehog signalling comprising the steps of:

(a) activating a cell of the immune system;

(b) contacting the cell with a candidate modulator;

(c) monitoring Hedgehog signalling;

(wherein steps (a), (b) and (c) can be carried out in any order); and

(d) determining whether the candidate modulator modulates Hedgehog signalling.

54. The method according to paragraph 53 wherein the cell of the immune system is a T-cell.

55. The method according to paragraph 54 wherein the T-cell is activated by activation of the T-cell receptor.

56. The method according to paragraph 55 wherein the T-cell receptor is activated with an antigen or antigenic determinant.

57. The method according to paragraph 55 wherein the T-cell receptor is activated by an anti-CD3 antibody.

58. The method according to paragraph 54 wherein the T-cell is co-activated.

59. The method according to paragraph 58 wherein the T-cell is co-activated by activation of CD28.

60. The method according to paragraph 58 wherein the T-cell receptor is co-activated by an anti-CD28 antibody.

61. A modulator identifiable by the method according to paragraph 53.

References

Ingham (1995) Curr Opin Genet and Dev 5:492-498

Chuang and McMahon (1999) Nature 397:617-621

Pepicelli et al (1998) Curr Biol 8(19):1083-1086

Hammerschmidt et al (1997) Trends Genet 13(1):14-21

Iemura et al (1998) PNAS 95:9337-9342

Kohler and Milstein (1975) Nature 256: 495-497

Davis et al Basic Methods in Molecular Biology (1986)

Sambrook et al Molecular Cloning: A Laboratory Manual, 2nd Edn, Cold Spring Harbor Laboratory Press, Cold Spring Harbor, N.Y. (1989)

Sussman et al (1994) Dev. Bio. 166:73-86

Leyns et al (1997) Cell 88(6):747-56

Bouwmeester et al (1996) Nature 382(6592):595-601

Hsu et al (1998) Mol Cell 1(5):673-83

Hsieh et al (1999) Nature 398(6726):431-6

Development (1997) 124:53-63

Cooper et al (1998) Science 280(5369):1603-7

Coventry et al (1998) Pediatr. Dev. Pathol. 1:29-41.

Hammerschmidt et al (1997) Trends Genet. 13:14.

Ingham (1998) EMBO J 17:3505.

Nüsslein-Volhard and Wieschaus (1980) Nature. 287:795.

Bumcroft et al (1995) Mol. Cell. Biol. 15:2294.

Lee et al (1994) Science. 266:1528.

Porter et al (1995) Nature. 374:363.

Williams et al (1999) J Cell Sci. 112:4405.

Zeng et al (2001) Nature. 411:716.

Murone et al (1999) Curr. Biol. 9:76.

Theil et al (1999) Cell. Tissue Res. 296:75.

Van den Brink et al (2001) Gastroenterol. 121:317.

Treier et al (2001) Development. 128:377.

Thomas (2000) Diabetes. 49:2039.

Fan and Khavari (1999) J. Cell Biol. 147:71.

Bhardwaj et al (2001) Nat. Immunol. 2:172.

Fujita et al (1997) Biochem. Biophys. Res. Commun. 238:658.

Kenney and Rowitch (2000) Mol. Cell. Biol. 20:9055.

Ruiz I Altaba (1999) Trends Genet. 15:418.

Outram et al (2000) Immunity. 13:187.

Vindelov and Christensen (1990) Methods Cell. Biol. 33:127.

Chtanova et al (2001) J. Immunol. 167:3057.

Granucci et al (2001) Eur J Immunol. 31:2539.

Fan et al (1997) Nat. Med. 3:788.

Strasser et al (1991) Cell. 67:889.

Katsumata et al (1992) Proc Natl Acad Sci USA. 89:11376.

Nakayama et al (1993) Science. 261:1584.

Veis et al (1993) Cell. 75:229.

Lees (1995) Curr. Op. Cell. Biol. 7:773.

Morgan (1995) Nature. 374:131.

Sherr (1994) Cell. 79:551.

Sherr (1996) Science. 274:1672.

Elledge (1996) Science. 274:1664.

Borgne et al (1999) J. Biol. Chem. 274:11977.

Peter et al (1990) Cell. 61:591.

Barnes et al (2001) EMBO J. 20:2214.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

We claim:

1. A method of modulating T-cell activation comprising contacting T-cells with a modulator of a Hedgehog signalling pathway or a modulator of a pathway which is a target of the Hedgehog signaling pathway.

2. The method according to claim 1, wherein the Hedgehog signalling pathway is the Sonic hedgehog, Indian hedgehog, or Desert hedgehog signalling pathway.

3. The method according to claim 1, wherein the pathway which is a target of the Hedgehog signaling pathway is the Wnt signaling pathway.

4. The method according to claim 1, wherein the modulator is an inhibitor or upregulator of the biological activity of the pathway.

5. The method according to claim 4, wherein the inhibitor is selected from the group consisting of HIP, cyclopamine, Frzb, Cerberus, WIF-1, Xnr-3, Gremlin, Follistatin or a derivative, fragment, variant, mimetic, homologue or analogue thereof, Ptc, Cos2, PKA, and an agent of the cAMP signal transduction pathway.

6. The method according to claim 1, wherein the modulator is selected from the group consisting of TGF-β family members, interleukins, IFN-γ, an FLT3 ligand, BMP superfamily members, antibodies, and small organic compounds.

7. The method according to claim 6, wherein the TGF-β family members are TGF-β-1 or TGF-β-2.

8. The method according to claim 6, wherein the interleukins are IL-4, IL-10, or IL-13.

9. A method of modulating T-cell proliferation comprising contacting T-cells with a modulator of a Hedgehog signalling pathway or a modulator of a pathway which is a target of the Hedgehog signalling pathway.

10. The method according to claim 9, wherein the Hedgehog signalling pathway is the Sonic hedgehog, Indian hedgehog, or Desert hedgehog signalling pathway.

11. The method according to claim 9, wherein the pathway which is a target of the Hedgehog signaling pathway is the Wnt signaling pathway.

12. The method according to claim 9, wherein the modulator is an inhibitor or upregulator of the biological activity of the pathway.

13. The method according to claim 12, wherein the inhibitor is selected from the group consisting of HIP, cyclopamine, Frzb, Cerberus, WIF-1, Xnr-3, Gremlin, Follistatin or a derivative, fragment, variant, mimetic, homologue or analogue thereof, Ptc, Cos2, PKA, and an agent of the cAMP signal transduction pathway.

14. The method according to claim 9, wherein the modulator is selected from the group consisting of TGF-β family members, interleukins, IFN-γ, an FLT3 ligand, BMP superfamily members, antibodies, and small organic compounds.

15. The method according to claim 14, wherein the TGF-β family members are TGF-β-1 or TGF-β-2.

16. The method according to claim 14, wherein the interleukins are IL-4, IL-1 0, or IL-13.

17. A method of modulating T-cell apoptosis comprising contacting T-cells with a modulator of a Hedgehog signalling pathway or a modulator of a pathway which is a target of the Hedgehog signalling pathway.

18. The method according to claim 17, wherein the Hedgehog signalling pathway is the Sonic hedgehog, Indian hedgehog, or Desert hedgehog signalling pathway.

19. The method according to claim 17, wherein the pathway which is a target of the Hedgehog signaling pathway is the Wnt signaling pathway.

20. The method according to claim 17, wherein the modulator is an inhibitor or upregulator of the biological activity of the pathway.

21. The method according to claim 20, wherein the inhibitor is selected from the group consisting of HIP, cyclopamine, Frzb, Cerberus, WIF-1, Xnr-3, Gremlin, Follistatin or a derivative, fragment, variant, mimetic, homologue or analogue thereof, Ptc, Cos2, PKA, and an agent of the cAMP signal transduction pathway.

22. The method according to claim 17, wherein the modulator is selected from the group consisting of TGF-β family members, interleukins, IFN-γ, an FLT3 ligand, BMP superfamily members, antibodies, and small organic compounds.

23. The method according to claim 22, wherein the TGF-β family members are TGF-β-1 or TGF-β-2.

24. The method according to claim 22, wherein the interleukins are IL-4, IL-10, or IL-13.
